(19) United States
(12) Patent Application Publication
(10) Pub. No.: US 2011/0160071 A1
(43) Pub. Date: Jun. 30, 2011

(54) Novel Proteins and Methods for Designing the Same

(76) Inventors: Brian M. Baynes, Cambridge, MA (US); Dasa Lipovsek, Cambridge, MA (US); Shaun Lippow, San Francisco, CA (US)

(21) Appl. No.: 12/995,549

(22) PCT Filed: Jun. 3, 2009
(86) PCT No.: PCT/US09/46185

§ 371 Date: Mar. 18, 2011

(30) Foreign Application Priority Data
Jun. 3, 2008 (US) 61/058557

(51) Int. Cl. C40B 30/02; C07K 14/00; C07H 21/00; C40B 50/02

(52) U.S. Cl. 506/8; 530/350; 536/23.1; 506/24

(57) ABSTRACT

Aspects of the invention relate to variant proteins and methods for designing and using the same. In some embodiments, the invention relates to methods for determining a functional variant of a protein that is restricted by one or more known legal rights, such as patent rights. Functional variants according to this invention are free of such restrictions.

### RELATED APPLICATIONS

This application claims the benefit under 35 U.S.C. §119(e) of U.S. provisional patent application Ser. No. 61/058,557, filed Jun. 3, 2008, the content of which is incorporated herein by reference in its entirety.

### FIELD OF THE INVENTION

Methods and compositions of the invention relate to novel proteins, protein variant libraries and methods of designing and using the same. More particularly, methods and compositions of the invention relate to novel protein variants that exhibit a desired characteristic (for example, a biological function, activity or structural feature) of a reference or parent protein that is associated with a legal restriction such as a patent right.

### BACKGROUND

In vitro protein evolution and selection methods (e.g., phage, yeast, mRNA, and ribosome display) have been used to identify proteins with desired functional properties, such as binding affinity for a macromolecular target or an enzymatic activity. Regardless of the method used, an initial step typically involves generating libraries of nucleic acids with sequences that encode polypeptides that are related to an original protein scaffold but that differ from an original protein sequence. Subsequently, each of the nucleic acids may be transcribed and translated into a corresponding protein. Associated nucleic-acid-protein complexes are then exposed to a target or substrate of interest, and those variants that bind the target with a desired affinity or that have a desired catalytic activity are isolated. Selected proteins can be produced on a large scale, typically in a microbial or mammalian-cell expression system, purified and used as affinity reagents, therapeutic proteins, or designer enzymes.

The new science of synthetic biology is predicated on the assumption that biological entities (e.g., genes, proteins and organisms) may be artificially constructed by specifying a molecular sequence and assembling a construct (e.g., a polynucleotide) on the basis of this sequence. For example, a polynucleotide is typically constructed by fabricating shorter segments of nucleotide bases or oligonucleotides and joining those segments together. Once the polynucleotide, such as, for example, a gene, is constructed, the polynucleotide may be incorporated into a vector and used to transfect a given cell line.

The underlying premise is that if a nucleotide sequence is specified, it may be constructed from shorter segments freely. However, nucleotide sequences may be protected in various ways. For example, certain oligonucleotides or peptides may be patented and not available for licensing. Thus, simply specifying a nucleotide sequence may be not sufficient if the underlying components are legally unavailable for use.

Further, many molecular segments have dangerous properties, require special handling or have other features (or use restrictions) that make them hard to use. Certain polynucleotides may be better used when introduced into certain vectors or cell types, and some materials may be unsuitable for use in products destined for certain members of the population. Therefore a need remains for improved methods of designing novel proteins that are free of pre-identified legal rights, yet exhibit a desired biological activity or function.

### SUMMARY OF THE INVENTION

Aspects of the invention relates to systems and methods for determining a functional variant of a protein that is subject to patent rights. As used herein, a restricted protein refers to a protein subject to, for example, legal or contractual restrictions, such as the restrictions imposed by patent rights on the making, using, selling, offering to sell or importing a protein or nucleic acids that encode such protein. Aspects of the invention involve identifying a restricted protein that exhibits a biological activity, the restricted protein being subject to a patent right; determining at least one feature of the restricted protein, wherein the patent right is contingent upon the feature; applying a computational design protocol to the restricted protein to generate a plurality of variant protein sequences that excludes any variant protein sequence that correspond to a variant protein having the feature; generating a plurality of nucleic acid molecules having predefined sequences encoding the plurality of variant proteins; expressing the nucleic acid molecules to produce a plurality of variant proteins; and screening the plurality of variant proteins for biological activity thereby to determine a functional variant of the restricted protein that is not subject to the patent right. As used herein, a patent right is a legal right for a rights-holder to exclude others from practicing the patented invention in connection with the making, using, offering for sale, selling, or importing the restricted protein.

In one embodiment, the restricted protein may have one or more structural characteristics associated with a biological activity. In a preferred embodiment, the method further comprises a step of determining at least one structural characteristic associated with the restricted protein. Such a structural characteristic may be correlated with a biological activity, and each of the variant proteins may comprises the structural characteristic. Patent rights may be contingent upon the presence or nature of a feature. For example, a feature may be an affirmative feature or a negative feature, a qualitative feature or a quantitative feature. A feature can comprise an aspect of a nucleic acid or amino acid sequence corresponding to the restricted protein, an aspect of a tertiary structure of the restricted protein, a biological activity exhibited in an in vitro assay, or molecular weight of the restricted protein. In some embodiments, the structural characteristic is qualitatively correlated with a level of biological activity exhibited by the restricted protein. Structural characteristic can comprise an aspect of a nucleic acid or amino acid sequence corresponding to the restricted protein, an aspect of a tertiary structure of the restricted protein. In some embodiments, the functional variant can exhibit a similar, a lower or a higher biological activity of the restricted protein. For example, the functional variant can exhibit at least about 10%, 20%, 30%, 40%, 50%, 60%, 70%, 80%, 90%, 95%, 100%, 110%, 120%, 130%, 140% or 150% of the biological activity of the restricted protein.

Aspects of the invention relate to the generation of high-density variant sequences libraries. A high-density variant sequence library may include more than about 100 different sequence variants (e.g., about 100, 1000, 2000, 3000, 4000, 5000, 6000, 7000, 8000, 9000, 10,000, 25,000, 50,000, 75,000, 100,000, 250,000, 500,000, 750,000, or 1,000,000 different sequences). Accordingly, aspects of the invention also relate to the generation of high-density nucleic acid molecules library. A high-density nucleic acid library may include more than about 100 different sequence variants (e.g., about 100, 1000, 2000, 3000, 4000, 5000, 6000, 7000, 8000, 9000, 10,000, 25,000, 50,000, 75,000, 100,000, 250,000, 500,000, 750,000, or 1,000,000 different molecules having pre-defined sequences). In a preferred embodiment, a high percentage of the different sequences are pre-defined sequences. For example, at least about 50%, 60%, 70%, 80%, 90%, 95% or 99% of the plurality of nucleic acid molecules correspond exactly with the pre-determined sequences.

Aspects of the invention provide methods for designing a novel protein having a predetermined functional property. In some embodiments, the designing strategy involves obtaining a sequence of a known protein wherein the known protein has at least one associated feature, identifying if the at least one feature is subject to patent rights, identifying a plurality of mutation, or variation, tolerant positions that do not affect the predetermined functional property, modifying the feature by substituting a plurality of amino acids at the mutation tolerant positions to generate a library a variants having alternate features that are not subject to the patent rights, screening the library of variants in silico to produce a rank ordered list of variants, generating nucleic acid molecules having predefined sequences that encode at least 10 variants, expressing the nucleic acid molecules to produce the protein variants and screening the variants to identify novel proteins having the predetermined functional property and not subject to patent rights. For example, the feature me be selected from the group of amino acid sequence, nucleic acid sequence, molecular weight, tertiary structure, and the like. In one embodiment, the invention provides a method for designing a novel protein having a predetermined biological activity and involves identifying a plurality mutation tolerant positions in a reference (or parent) protein having a known biological activity by comparing its amino acid sequence to the amino acid sequences of a plurality of related proteins having the same biological activity; and substituting at least one amino acid present at the mutation tolerant positions to produce a novel protein that has an amino acid sequence that is different to the reference protein. In a further embodiment, the invention provides a method for designing a novel protein having a predetermined functional property and involves obtaining a sequence of a reference protein having the predetermined functional property, identifying a plurality of mutation tolerant positions in the reference protein by comparing its amino acid sequence to the amino acid sequences of a plurality of related proteins having the same functional property, and substituting at least one amino acid present at the mutation tolerant positions to produce a novel protein that has an amino acid sequence that is different to the reference protein.

In the event that the crystal structure of a protein is known, the amino acids that are implicated in the activity of the reference protein can be inferred. If only the primary structure of the reference protein is known a three-dimensional structure can be modeled using computational protein modeling software. Accordingly, in some embodiments, the method comprises obtaining a three dimensional model of the reference protein, identifying a plurality mutation tolerant positions in the reference protein by determining amino acids not involved in the active site, and substituting at least one amino acid present at the mutation tolerant position to produce a novel protein that has the known predetermined activity. In some embodiment, the reference sequence is compared to a plurality of related proteins having the same biological activity by aligning the reference protein and related protein amino acids sequences to make a sequence alignment. In a preferred embodiment, the reference protein and the related proteins have at least about 30% sequence identity. In another embodiment, the amino acid sequence of a variable region of the reference protein is compared to the variable region of the related proteins and the substitutable positions (i.e. mutation tolerant positions) is in the variable region.

In some embodiments, identifying a plurality mutation tolerant positions may be identified in a reference protein having a known biological activity by comparing its amino acid sequence (or structure) to the amino acid sequences (or structure) of a plurality of related proteins having the same biological activity. Aspects of the invention also provides a method for designing a novel protein having a predetermined biological activity, involving obtaining a sequence of a reference protein having the predetermined biological activity, screening a plurality a possible variants in silico to produce a rank ordered list of variants and substituting the amino acids present at the highest ranked mutation tolerant positions to produce a first library of proteins variants having an amino acid sequence that is different to the reference protein. In further embodiment, nucleic acid molecules that encode at least a subset, for example, about 10, 20, 30, 40 or more, of the protein variants are generated and expressed to produce the protein variants. The first library of novel proteins is then screened for the predetermined functional property and a first set of novel proteins having the least homology to the reference protein and the highest predetermined biological activity is selected. In a further embodiment, the first set of novel proteins is screened in silico to produce a rank ordered list of variants, the amino acids present at the highest ranked mutation tolerant positions can be substituted to produce a library of proteins having an amino acid sequence that is different to the reference protein and to the first library of protein variants, nucleic acid molecules that encodes at least a subset of the protein variants are generated and expressed to produce the protein variants. Protein variants may be screened for the predetermined functional property and a second set of novel proteins having the least homology to the reference protein and the highest predetermined biological activity is selected. The process can be reiterated to select a third set of novel proteins having the least homology to the reference protein and the highest predetermined biological activity. For example, the novel protein has less than about 95%, 90%, 80%, 70%, 60% homology to the reference protein sequence. The least homology is, for example, no less than 90%, 80%, 70%. One should appreciate that the novel protein can have similar, higher or lower biological activity compared to the reference protein. For example, the novel protein has at least about 95%, 90%, 85%, 80%, 75%, 70%, 60% or 50% of the reference protein functional property or biological activity.

Aspects of the invention are useful for designing novel protein having similar structural properties than reference proteins for example similar thermostability, solubility or expression level and substitution in a mutation tolerant position does not reduce the protein functional property, biological activity, stability, solubility, and expression level. In some embodiments, the mutation tolerant position correspond to solvent-accessible amino acids, amino-acids at least a pre-determined distance from the active site, amino acids not involved in stabilizing secondary, tertiary or quaternary protein structure.

In some aspects, the invention provides method of designing a library of variant proteins, the method comprising identifying a reference protein that exhibits a biological activity, determining at least one qualitative feature of the reference protein; the qualitative feature being divisible into at least a first and a further constrained second gradient level, applying to the reference protein a design algorithm to generate a plurality of variant protein sequences that comprise the qualitative feature corresponding to the first gradient level, generating a plurality of nucleic acid molecules having predefined sequences encoding the plurality of variant proteins, expressing the nucleic acid molecules to produce the variant proteins and screening the variant proteins for biological activity to identify a functional variant protein exhibiting the biological activity. The steps of applying, generating and expressing may be repeated with the functional variant protein as the reference protein and using a design algorithm to generate a second plurality of variant protein sequences that comprise the qualitative feature corresponding to the second gradient level and screening the second plurality of variant protein sequences to identify a functional variant protein exhibit the biological activity and have the qualitative feature corresponding to the second gradient level. In a further embodiment, the applying, generating expressing and screening steps may be repeated with further constrained levels of the qualitative feature until a functional variant protein with target level of the qualitative feature is determined.

Aspects of the invention relate to protein libraries that can be used to evaluate, screen, or select polypeptides of interest. In some embodiments, the invention relates to expression libraries that can be used to screen or select for polypeptides having one or more functional and/or structural properties (e.g., one or more predetermined catalytic, enzymatic, receptor-binding, therapeutic, or other properties). Aspects of the invention provide expression libraries (e.g., nucleic-acid/polypeptide libraries) that are enriched for candidate polypeptides lacking one or more unwanted characteristics. For example, a library that expresses many different polypeptide variants may be designed to exclude polypeptides that have poor in vivo solubility, high immunogenicity, low stability, etc., or any combination thereof. Furthermore, a library of protein variants may be designed to exclude a feature upon which a pre-identified patent right is contingent—i.e., to produce variants that are not restricted by such patent right. Accordingly, aspects of the invention provide methods of generating filtered expression libraries that are enriched for candidate molecules having physiologically compatible or desirable characteristics, or lacking certain undesired features. In some embodiments, a filtered expression library may be screened and/or exposed to selection conditions to identify one or more polypeptides having a function or structure of interest.

Accordingly, aspects of the invention may be used to screen or select filtered libraries for target polypeptides of interest that also have desirable in vivo traits. Whereas selection methods using un-filtered libraries may yield proteins with required binding or catalytic properties, they generally do not select for other desirable properties. For example, proteins selected using un-filtered libraries frequently are found to have unacceptably low stability or solubility when purified and characterized. In the case of proteins designed for therapeutic applications, such as antibodies, antibody fragments, non-antibody target-binding proteins, and modified hormones or receptors, a common problem is that proteins selected from un-filtered libraries often evoke an immune response when introduced into patients, causing either inactivation of the putative therapeutic or adverse side effects.

In some embodiments, filtering techniques of the invention can be used to identify nucleic acid sequences to be included in a polypeptide expression library. In some embodiments, filtering techniques of the invention can be used to identify nucleic acid sequences to be excluded from a polypeptide expression library. In some embodiments, methods of the invention are useful for screening nucleic acid sequences that are candidates for inclusion in an expression library and identifying those sequences that encode polypeptides with one or more undesirable properties (e.g., poor solubility, high immunogenicity, low stability, etc.). Accordingly, aspects of the invention may be used to design a library of nucleic acids that encode a plurality of polypeptides having one or more biophysical or biological properties that are known or predicted to be within a predetermined acceptable or desirable range of values.

According to another embodiment, a method is provided for producing an unrestricted variant protein. Methods of the invention may comprise providing a structural model and an amino acid sequence for a reference protein having an desired characteristic; determining from the structural model and amino acid sequence at least one amino acid residue that is not correlated with said desired characteristic; and generating at least one variant protein by introducing a mutation at said at least one amino acid residue of the reference protein. In one aspect, the reference protein is a restricted by a proprietary right that is contingent upon a feature of said reference protein, and the feature is altered upon mutation of said at least one amino acid residue, thereby to produce a variant protein that is unrestricted by the proprietary right. The invention further provides for screening the variant protein for the desired characteristic.

In yet another embodiment, a method is provided for generating a library of unrestricted variant proteins. Methods of the invention may comprise, the method comprising providing a structural model and an amino acid sequence for a reference protein having an desired characteristic, said reference protein being a restricted by a proprietary right that is contingent upon a feature of said reference protein; determining from the structural model and amino acid sequence a plurality of mutation-tolerant amino acid residues that are not correlated with said desired characteristic; and generating a plurality of variant proteins by different mutations at least a subset of said mutation-tolerant amino acid residues of the reference protein. In one aspect, the feature is altered upon mutation of one or more of said mutation-tolerant amino acid residues, thereby to produce a library of variant proteins that is unrestricted by the proprietary right. Methods of the invention further contemplate screening the plurality of variant proteins for the desired characteristic, and optionally identifying at least one of the plurality of variant proteins a desired characteristic that is substantially equivalent to the reference protein.

Aspects of the invention also relate to methods of assembling libraries containing nucleic acids having predetermined sequence variations. In some embodiments, a library may be designed and assembled to be representative of a plurality of predetermined nucleic acid or polypeptide sequences that are selected (e.g., using a sequence filter of the invention) or provided (e.g., provided by a customer). In some embodiments, a library contains a plurality of related nucleic acids that include predetermined sequence differences at only a subset of positions.

A library assembly reaction may include a polymerase and/or a ligase. In some embodiments the assembly reaction involves two or more cycles of denaturing, annealing, and extension conditions. In some embodiments, the library nucleic acid may be amplified, sequenced or cloned after it is made. In some embodiments, a host cell may be transformed with the assembled library nucleic acid. Library nucleic acid may be integrated into the genome of the host cell. In some embodiments, the library nucleic acid may encode a polypeptide. The polypeptide may be expressed (e.g., under the control of an inducible promoter). The polypeptide may be isolated or purified. A cell transformed with an assembled nucleic acid may be stored, shipped, and/or propagated (e.g., grown in culture).

In another aspect, the invention provides methods of obtaining nucleic acid or protein libraries by sending sequence information and delivery information to a remote site. The sequence information may be analyzed at the remote site. Starting nucleic acids may be designed and/or produced at the remote site. The starting nucleic acids may be assembled in a process that generates the desired sequence variation at the remote site. In some embodiments, the starting nucleic acids, an intermediate product in the assembly reaction, and/or the assembled nucleic acid library may be shipped to the delivery address that was provided.

Other aspects of the invention provide systems for designing starting nucleic acids and/or for assembling the starting nucleic acids to make a target library. Other aspects of the invention relate to methods and devices for automating a multiplex oligonucleotide assembly reaction to generate a library of interest. Further aspects of the invention relate to business methods of marketing one or more protocols, systems, and/or automated procedures that involve sequence filtering and/or nucleic acid library assembly. Yet further aspects of the invention relate to business methods of marketing one or more libraries (e.g., one or more filtered libraries).

Further, aspects of the invention provide methods and systems for evaluating, designing, assembling, testing, and/or licensing constructs that may be used for biological applications. In some embodiments, constructs may be polynucleotide polymers. In certain embodiments, constructs may be polypeptide polymers. Aspects of the invention relate to analyzing one or more segments of a construct and identifying whether any use restrictions based on one or more rights restrictions (e.g., rights restrictions such as legal, business, and/or other rights restrictions) and/or one or more other features (e.g., structural, functional, and/or other properties) that may form the basis of a design, assembly, application, or other restriction are associated with the segment(s). Restrictions and/or features that are identified may provide information for design, assembly, application, and/or business decisions relating to the construct. One or more aspects of the invention may be computer-implemented, for example, so that a user can access an automated or partially automated system for analyzing a construct to provide information and/or decisions relating to one or more design, development, manufacturing, and/or other business options that may be helpful to the user. A system of the invention may include a data repository comprising use restriction and/or feature information associated with one or more molecular segments (e.g., polynucleotide or polypeptide segments) that can be used as building blocks for larger constructs. A data repository also may include other technical, legal, and/or business information relating to in vitro and/or in vivo applications for constructs and/or construct segments of interest. For example, information relating to therapeutic, agricultural, industrial, research, and/or environmental applications may be provided. Such information may relate to cell lines, organisms, biological assays, chemical assays, packaging, therapeutic compositions, production details, metabolic pathways, etc., or any combination thereof. In some embodiments, rights restrictions related to fabricating a construct (e.g., relating to the chemical synthesis, in vitro amplification, assembly, expression, cloning, etc., of one or more oligo- or polynucleotides or peptides) may be provided in a system or data repository of the invention.

Applicants have appreciated that in addition to the biological constraints imposed by the scientific problem being solved, there may be many other considerations that may impact the ability of a bioengineer to make a desired construct. After laboring on the design of the construct, the bioengineer is left to the difficult task of ascertaining what, if any, restrictions exist on the use of each of the proposed molecular segments in the construct. Further, the bioengineer must determine what other considerations will arise in connection with each of the proposed molecular segments and what precautions might be required. Typically, the bioengineer must find the information that he or she needs by hand, accessing many different and unrelated sources. If the bioengineer discovers that one or more proposed molecular segments are not suitable for use in the designed construct, the bioengineer must search for an alternative or replacement molecular segment. The process for “clearing” a molecular segment for use in a construct is not only labor-intensive, but also inefficient, time-consuming, and prone to errors and oversights.

Applicants have further appreciated that biology is characterized by significant intellectual property barriers. In the cases in which biological intellectual property is cross-licensed, it is in an ad hoc manner, requiring fresh negotiations for each piece of intellectual property to be licensed.

Aspects of the invention provide an organized system for analyzing and “clearing” construct segments and final constructs that a user intends to assemble. For example, some embodiments of the present invention provide an efficient marketplace for biological intellectual property rights.

Other embodiments of the invention relate to a method and system for providing information about constructs that are useful for biological applications, and/or about the building blocks that can be assembled to form the constructs. It should be appreciated that constructs or building blocks may be naturally-occurring or synthetic. Further, synthetic constructs may be designed and/or engineered to have naturally-occurring properties (e.g., naturally occurring polynucleotide or polypeptide sequences) once they are fabricated. However, synthetic constructs also may be designed and/or engineered to have non-naturally occurring characteristics (e.g., non-naturally occurring sequence variants, or non-natural combinations of functional elements). It also should be appreciated that the terms constructs and building blocks are relative terms. For example, in the context of a polynucleotide or polypeptide polymer, a building block may be a shorter segment of the polynucleotide or polypeptide polymer. However, the polynucleotide or polypeptide polymer itself may be used as a building block for a larger polynucleotide or polypeptide polymer. Embodiments of the invention provide a method and system for determining use restrictions and/or other features associated with constructs and/or smaller building blocks (e.g., molecular segments) that each can be used alone or in suitable combination to assemble multicomponent biological and/or synthetic devices and systems. Further, embodiments of the invention provide a method and system for identifying constructs and/or smaller building blocks having a defined feature set as candidates for a predetermined application specified by a user (e.g., for use in a predetermined biological system, for example, a recombinant cell).

Accordingly, aspects of the invention relate to a system and method for aiding in the fabrication of biological constructs. In one aspect the system includes a library aggregating a plurality of intellectual property rights relating to fabricating biological constructs; a licensing module licensing the intellectual property rights required to make the specific construct for a fee; and an accounts receivable module receiving the fee from a potential maker of the specific construct. In one embodiment, the system includes an accounts payable module distributing remuneration to the holders of the intellectual property rights required to make the specific construct. In another embodiment the system further includes a design module defining the steps of the process and the materials by which the specific construct is to be fabricated. In still another embodiment the system further includes a fabrication module utilizing the defined steps of the process and the materials by which the specific construct is to be fabricated in order to fabricate the specific construct.

In yet another embodiment, the system further includes a testing module for testing the fabricated specific construct against a predetermined criterion. The design module is utilized to re-define the steps of the process and the materials by which the specific construct is to be fabricated if the fabricated specific construct does not meet the predetermined criterion. In still yet another embodiment the library of aggregated intellectual property rights are aggregated from a plurality of intellectual property rights holders. In another embodiment, the design module is a computer aided design (CAD) module. In yet another embodiment, the library aggregating a plurality of intellectual property rights relating to fabricating biological constructs; the licensing module licensing the intellectual property rights required to make the specific construct for a fee; the accounts receivable module receiving the fee from a potential maker of the specific construct; the accounts payable module distributing remuneration to the holders of the intellectual property rights required to make the specific construct; the design module defining the steps of the process and the materials by which the specific construct is to be fabricated; and the fabrication module utilizing the defined steps of the process and the materials by which the specific construct is to be fabricated in order to fabricate the specific construct are controlled by a single entity.

In another aspect, the invention relates to a method for aiding in the fabrication of a specific biological construct. In one embodiment, the method includes the steps of aggregating a plurality of intellectual property rights relating to fabricating biological constructs; licensing the intellectual property rights required to make the specific construct for a fee; and receiving the fee from the potential maker of the specific construct. In one embodiment, the method includes distributing remuneration to the holders of the intellectual property rights required to make the specific construct. In another embodiment, the method includes the steps of defining the steps of the process and the materials by which the specific construct is to be fabricated. In another embodiment, the method further includes the steps of utilizing the defined steps of the process and the materials by which the specific construct is to be fabricated in order to fabricate the specific construct. In still yet another embodiment, the method includes the steps of testing the fabricated specific construct against a predetermined criterion; and re-defining the steps of the process and the materials by which the specific construct is to be fabricated if the fabricated specific construct does not meet the predetermined criterion. In still yet another embodiment, the defining of the steps of the process and the materials by which the specific construct is to be fabricated is performed with a computer aided design system. In another embodiment, the library of aggregated intellectual property rights are aggregated from a plurality of intellectual property rights holders. In one embodiment, the steps of licensing the intellectual property rights required to make the specific construct for a fee and the receiving of the fee from the potential maker of the specific construct is performed once for the specific construct. In another embodiment, the method includes the step of collaboratively marketing the specific construct. In still yet another embodiment, the method includes the step of collaboratively marketing a therapeutic or a diagnostic product identified using the specific construct. In a further embodiment, the method further includes the steps of identifying a therapeutic or diagnostic product using the specific construct; and collaboratively marketing the therapeutic or a diagnostic product.

Another aspect of the invention also relates to a clearinghouse which comprises a source of information about biological parts for the construction of synthetic biological constructs. More particularly, one embodiment of the invention provides a system for determining legal rights and/or other features associated with defined biological building blocks that can be used in combination to assemble many-component biological devices and systems. In addition, some embodiments of the invention provide a system for identifying biological parts or building blocks that have a defined feature set as candidates for use in a construct.

One embodiment of the invention provides methods and devices useful in computer aided design of a construct. According to this embodiment, a method for computer aided design of a multimeric construct comprises defining a feature set of biological parts, such as molecular DNA segments, that is suitable for use in the construct. Such a feature set includes public, private, or contractual use restrictions (or notation of lack thereof) on biological parts, such as patent restrictions, transfer restrictions, commercialization restrictions, safety restrictions, governmentally imposed restrictions, and field of use restrictions. By way of example, the data may provide notification that: use of a part requires a license, and may specify license terms in various contexts; the part must be used in a facility having some special level of biological containment; use of the part in combination with some other class of parts may constitute patent infringement; etc. The feature set may and typically will also include one or more characteristics, properties, values or attributes of the parts. For example, a feature set may comprise a characteristic related to function, utility, source (e.g., species, experimental system, etc.), cell-type specific and/or species-specific properties (e.g., expression, stability, toxicity, susceptibility to cell-type or species specific nucleases or proteases, etc.), interoperability with other parts or segments, nucleic acid sequence, amino acid sequence, codon usage, molecular weight, tertiary structure, quaternary structure, mRNA secondary structure, post-translational modifications, reactivity, modification sites, modes of detection, polarity, solubility properties such as hydrophobicity/hydrophilicity, membrane permeability, stability, bioavailability, safety, toxicity, isoelectric point, charge, thermostability, melting temperature, annealing temperature, catalytic activity, side groups, topology, kinetic complexity, immunogenicity, environmental hazards, and any combination of any of the foregoing, or other features. One or more of the characteristics of the feature sets described herein may provide a use restriction at any stage (e.g., design, assembly, application, testing, etc.) relating to the constructs described herein. For example, one or more of the features may form the basis of a determination that a construct has one or more undesirable properties. For example, in some embodiments, a user may specify a specific threshold level for each of one or more features or characteristics described herein (e.g., structural and/or functional properties), above which constructs are identified as being undesirable. In certain embodiments, a user may specify a specific threshold level for each of one or more features or characteristics described herein (e.g., structural and/or functional properties), below which constructs are identified as being undesirable. It should be appreciated that a system of the invention may provide feature information for construct building blocks taken alone and/or for combinations of two or more construct building blocks.

In some embodiments, a system of the invention may include a macro or routine (e.g., any suitable computer code) that can be accessed by a user to design a construct (e.g., a sequence) for expression in one or more user-specified cell type or species (e.g., from a list of available cell types or species provided by the system). In some embodiments, the macro or routine may be used to convert sequences (e.g., nucleic acid and/or protein sequence) of a designed construct or set of constructs to be optimized for replication and/or expression in one or more selected cell types and/or species. In some embodiments, different restrictions (e.g., rights restrictions, restrictions based on structural, functional, and/or other characteristics described herein, or any combination thereof) may be identified from the data repository for different cells types and/or species. Accordingly, a designer may use a system of the invention to determine which species and/or cell types to use in connection with one or more constructs of interest. In some embodiments, a user may use aspects of the invention to determine which species and/or cell types one or more constructs should be designed and/or fabricated for (e.g., based on patent rights, other use restrictions, expression properties, structural properties, functional properties, toxicity, etc., or any combination thereof in different cell types and/or species).

In one embodiment, the method further comprises searching a database, and/or collection of public and/or private databases, that comprises a plurality of molecular segment building blocks and a plurality of features. Each of the molecular segments may be associated with at least one feature. According to one aspect of the invention, the method comprises determining from the database a molecular segment that is suitable for use in the construct as one having the defined feature set.

According to another embodiment of the invention, a first molecular segment building block, or combination of building blocks, is defined, and a database is searched. The database may comprise a plurality of molecular segments and a plurality of features, each of the plurality of molecular segments being associated with at least one feature. In one embodiment, a first feature set that is associated with the first molecular segment is determined. Optionally, in another embodiment, a second molecular segment building block, or combination or building blocks, having a second feature set that is an alternative to the first feature set is determined as an alternative molecular segment for use in the construct. According to one aspect of the invention, molecular segment building blocks may comprise one or more nucleobases, natural nucleotides, unnatural nucleotides, nucleotide analogs, modified nucleotides, codons, nucleic acids, oligonucleotides, polynucleotides, natural amino acids, unnatural amino acids, amino acid analogs, modified amino acids, peptides, polypeptides, chemical moieties, small molecules, vectors, plasmids, restriction sites, primers, hybridization sites, selection markers, detection markers, linkers, labels, ligands, antigens, and antibodies or fragment thereof. Generally, aspects of the invention can be applied to building a gene or a protein from subparts such as oligonucleotides or oligopeptides, a transcription unit (an open reading frame plus regulatory elements), assemblies of multiple genes, vectors, chromosomes, genomes, and cells, all from smaller bioparts. In another embodiment, building blocks may comprise a combination of any one or more of the foregoing. For example, in an oligonucleotide construct, a nucleotide analog linked to a detection marker may be considered to be a single molecular segment, or may be considered to two or three molecular segments (i.e., the detection marker, the nucleotide analog, and the chemical linker). As other examples, the biopart may be a 50 Kb DNA polynucleotide encoding and controlling expression of a group of enzymes that catalyze formation of an organic molecule, or may be a cell for addition to a culture which has a complementary function, e.g., secretes a nutrient necessary for survival of other cells in the culture. Accordingly, nucleic acid or polypeptide building blocks may be polymers each having about 4 to 10; about 10 to 50; about 50 to 100; about 100 to 1,000; about 1,000 to 10,000, or fewer or more nucleotide or amino acid monomers, respectively.

Another aspect of the invention relates to a method for determining the rights associated with the use of molecular segment building blocks in a construct. In one embodiment, the method includes the steps of: defining a molecular segment for use in the construct; and searching a database for rights associated with the defined molecular segment. In this embodiment, the database includes a plurality of molecular segments and a plurality of rights, each right of the plurality of rights associated with at least one of the plurality of molecular segments. In another embodiment, the method further comprises the step of displaying the rights associated with the molecular segment. In yet another embodiment, the construct includes a polynucleotide and the molecular segment comprises an oligonucleotide or smaller polynucleotide, e.g., an open reading frame or portion thereof, or a regulatory segment. According to another embodiment, the method further includes the step of decomposing the construct into a plurality of building blocks. In another embodiment, the method further includes the step of identifying an alternate building block if the rights associated with the defined building block do not reach a predetermined specification. In yet another embodiment, the rights in the database are selected from a group consisting of patent restrictions, transfer restrictions, commercialization restrictions, safety restrictions, governmentally imposed restrictions, and field of use restrictions. Another aspect of the invention provides a system for determining the rights associated with the use of molecular segments in a construct. In one embodiment, the system includes a molecular segment module defining a molecular segment for use in the construct; a database including a plurality of molecular segments and a plurality of rights, each right of the plurality of rights associated with at least one of the plurality of molecular segments; a database manager for searching the database for the defined molecular segment and a display displaying rights associated with the defined molecular segment in response to the search of the database. In one embodiment, the construct includes a polynucleotide and the molecular segment comprises an oligonucleotide or smaller polynucleotide, e.g., an open reading frame or portion thereof, or a regulatory segment, or any other selected polynucleotide segment.

In one embodiment, the system further includes a construct decomposer for decomposing a construct into a plurality of molecular segments (e.g., 2, 3, 4, 5, about 5 to 10, about 10 to 20, about 20 to 50, about 50 to 100, or more different molecular segments). In yet another embodiment, the system further includes an alternative molecular segment identifier for identifying an alternate molecular segment if the rights associated with the defined molecular segment are incompatible with one or more other segments in the construct, fail to meet some criteria, or do not reach a predetermined level. The predetermined level may be, for example, no associated rights, so the molecular segment is freely available for use, or third party ownership but available for use under a license agreement. In yet another embodiment, the rights in the database are selected from a group consisting of patent restrictions, transfer restrictions, commercialization restrictions, safety restrictions, governmentally imposed restrictions, and field of use restrictions.

In another aspect, the invention relates to a database including a first plurality of records, each of the first plurality of records corresponding to a respective one of a plurality of molecular structures; and a second plurality of records, each of the second plurality of records corresponding to a respective one of a plurality of rights, wherein each of the plurality of first records is associated with at least one of the plurality of second records. In one embodiment, a database includes a compilation comprising information, documents, records and/or files, while in another embodiment, a database comprises electronic links or hyperlinks to information, documents, records, and/or files.

In a further embodiment, the invention provides a method for obtaining a right to use a building block such as a molecular segment in a construct comprising defining a molecular segment for use in a construct and searching a database. The database comprises a plurality of molecular segments and an associated plurality of use restrictions. Each of the plurality of use restrictions is associated with at least one part or molecular segment. In one embodiment, the database also includes at least one form license to use a part or molecular segment associated with a use restriction. According to one aspect of the invention, the method further comprises identifying a use restriction associated with the defined part or molecular segment. In one embodiment, optionally, a form license to use the defined part or molecular segment is accessed and, if desired, made available for inspection and execution.

In another embodiment, the database may comprise annotations in addition to rights and specifications associated with a part or molecular segment, such as literature references, attributions, publications, patent references, purchasing information, and/or ordering capabilities. Embodiments of the invention provide a functionality to access an on-line or otherwise remotely accessible repository/collection of extensively annotated biological parts offered for sale by a proprietor. In this respect, the U.S. application Ser. No. 09/996,649, METHODS AND SYSTEMS FOR DESIGNING MACHINES INCLUDING BIOLOGICALLY-DERIVED PARTS, (WO/02/1034661), which is incorporated herein by reference, can be referred to.

It is contemplated that diverse researchers could choose to deposit voluntarily their biological discoveries and creations, or the sequence information defining them, with the repository, which would act as a distributor to interested researchers and scientists. The researchers could specify the structure, sequence, use restrictions, royalty loads, compatibility data, functional data, etc. of his or her created or discovered biological part. Accordingly, a system or data repository of the invention also may enable a user to submit information (e.g., relating to restrictions, structural properties, functional properties, etc.) that the user determined based on the assembly, analysis, and/or use of one or more constructs and/or construct building blocks alone or in combination with one or more additional constructs and/or construct building blocks. This information may be monitored, checked, and/or annotated by a system administrator. The information may include any type or information including, for example, technical data. For example, the information may include one or more descriptions and/or data sets relating to the interaction of one or more different constructs or building blocks (e.g., molecular segments—for example, different functional and/or structural domains or motifs) under different conditions, when combined with other constructs or building blocks (e.g., molecular segments), when cloned into certain vectors, when expressed in certain cells, when expressed in a host cell in the presence of one or more genomic mutations, when expressed or replicated in a host cell in the presence of one or more other constructs and/or building blocks (e.g., molecular segments), etc., or any combination thereof. The information may include one or more links to a remote site (e.g., a public database) where information may be stored. Accordingly, the content of a system and/or data repository of the invention may be enhanced as additional information is provided by users.

In some aspects of the invention, a repository may be complemented by a clearinghouse function, and optionally might manufacture polynucleotides, proteins, or cells for its inventory and/or to the specifications of a customer. The repository/clearinghouse may also provide on-line bioconstruct design aids, access to simulation software for virtual testing of constructs, and information regarding downstream use of bioparts.

It should be understood that the embodiments above-mentioned and discussed below are not, unless context indicates otherwise, intended to be mutually exclusive. Other features and advantages of the invention will be apparent from the following detailed description, and from the claims. The claims provided below are hereby incorporated into this section by reference.

### BRIEF DESCRIPTION OF THE FIGURES

FIG. 1 shows one embodiment of a plurality of oligonucleotides that may be assembled in a polymerase-based multiplex oligonucleotide assembly reaction;

FIG. 2 illustrates certain aspects of an embodiment of sequential assembly of a plurality of oligonucleotides in a polymerase-based multiplex assembly reaction;

FIG. 3 illustrates an embodiment of a ligase-based multiplex oligonucleotide assembly reaction;

FIG. 4 illustrates several embodiments of ligase-based multiplex oligonucleotide assembly reactions on supports;

FIG. 5 outlines an embodiment of a method of filtering expression library sequences;

FIG. 6 outlines an embodiment of a method of assembling a nucleic acid library containing predetermined nucleic acid sequence variants;

FIG. 7 illustrates an embodiment of an assembly technique for producing a pool of predetermined nucleic acid sequence variants;

FIG. 8 is a schematic block diagram illustrating a system according to embodiments of the invention;

FIG. 9 is a schematic diagram illustrating an exemplary computing environment on which embodiments of the invention can be implemented;

FIG. 10 is a schematic diagram illustrating an example of data structures used in the design phase and rights management phase modules of FIG. 1 in accordance with one embodiment;

FIG. 11 is a schematic diagram illustrating a construct decomposing capability according to embodiments of the invention;

FIG. 12 is a flowchart illustrating a method for design of constructs according to embodiments of the invention;

FIG. 13 illustrates a non-limiting embodiment of a method for designing assembly nucleic acids and an assembly strategy for a precise high-density nucleic acid library; and

FIG. 14 illustrates non-limiting embodiments of assembly techniques in panels A-D;

### DETAILED DESCRIPTION OF THE INVENTION

Aspects of the invention relate to systems and methods for determining a functional variant of a protein that is subject to patent rights. As used herein, a restricted protein refers to a protein subject to, for example, legal or contractual restrictions, such as the restrictions imposed by patent rights on the making, using, selling, offering to sell or importing a protein or nucleic acids that encode such protein. Aspects of the invention involve identifying a restricted protein that exhibits a biological activity, the restricted protein being subject to a patent right; determining at least one feature of the restricted protein, wherein the patent right is contingent upon the feature; applying a computational design protocol to the restricted protein to generate a plurality of variant protein sequences that excludes any variant protein sequence that correspond to a variant protein having the feature; generating a plurality of nucleic acid molecules having predefined sequences encoding the plurality of variant proteins; expressing the nucleic acid molecules to produce the plurality of variant proteins; and screening the plurality of variant proteins for biological activity thereby to determine a functional variant of the restricted protein that is not subject to the patent right. As used herein, a patent right is a legal right for a rights-holder to exclude others from practicing a patented invention in course of making, using, offering for sale, selling, or importing the restricted protein.

In one embodiment, the restricted protein may have one or more structural characteristics associated with a biological activity. In a preferred embodiment, the method further comprises a step of determining at least one structural characteristic associated with the restricted protein. Such a structural characteristic can be correlated with a biological activity and the plurality of variant proteins generated comprises the structural characteristic. Patent rights may be contingent upon the presence or nature of a feature. For example, a feature may be an affirmative feature or a negative feature, a qualitative feature or a quantitative feature. A feature can comprise an aspect of a nucleic acid or amino acid sequence corresponding to the restricted protein, an aspect of a tertiary structure of the restricted protein, a biological activity exhibited in an in vitro assay, or molecular weight of the restricted protein. In some embodiments, the structural characteristic is qualitatively correlated with a level of biological activity exhibited by the restricted protein. Structural characteristic can comprise an aspect of a nucleic acid or amino acid sequence corresponding to the restricted protein, an aspect of a tertiary structure of the restricted protein. In some embodiments, the functional variant can exhibit a similar, a lower or a higher biological activity of the restricted protein. For example, the functional variant can exhibit at least about 10%, 20%, 30%, 40%, 50%, 60%, 70%, 80%, 90%, 95%, 100%, 110%, 120%, 130%, 140% or 150% of the biological activity of the restricted protein.

Aspects of the invention relate to the generation of high-density variant sequences libraries. A high-density variant sequence library may include more than about 100 different sequence variants (e.g., about 100, 1000, 2000. 3000, 4000, 5000, 6000, 7000, 8000, 9000, 10,000, 25,000, 50,000, 75,000, 100,000, 250,000, 500,000, 750,000, or 1,000,000 different sequences). Accordingly, aspect of the invention also relates to the generation of high-density nucleic acid molecules library. A high-density nucleic acid library may include more than about 100 different sequence variants (e.g., about 100, 1000, 2000. 3000, 4000, 5000, 6000, 7000, 8000, 9000, 10,000, 25,000, 50,000, 75,000, 100,000, 250,000, 500,000, 750,000, or 1,000,000 different molecules having pre-defined sequences). In a preferred embodiment, a high percentage of the different sequences are pre-defined sequences. For example, at least about 50%, 60%, 70%, 80%, 90%, 95% or 99% of the plurality of nucleic acid molecules correspond exactly with the pre-determined sequences.

Aspects of the invention provide methods for designing a novel protein having a predetermined functional property. In some embodiments, the design strategy involves obtaining a sequence of a known protein wherein the known protein has at least one associated feature, identifying if the at least one feature is subject to patent rights, identifying a plurality of mutation tolerant positions that do not affect the predetermined functional property, modifying the feature by substituting a plurality of amino acids at the mutation tolerant positions to generate a library a variants having alternate features that are not subject to the patent rights, screening the library of variants in silico to produce a rank ordered list of variants, generating nucleic acid molecules having predefined sequences that encode at least 10 variants, expressing the nucleic acid molecules to produce the protein variants and screening the variants to identify novel proteins having the predetermined functional property and not subject to patent rights. For example, the feature me be selected from the group of amino acid sequence, nucleic acid sequence, molecular weight, tertiary structure, etc. In one embodiment, the invention provides a method for designing a novel protein having a predetermined biological activity and involves identifying a plurality mutation tolerant positions in a reference (or parent) protein having a known biological activity by comparing its amino acid sequence to the amino acid sequences of a plurality of related proteins having the same biological activity; and substituting at least one amino acid present at the mutation tolerant positions to produce a novel protein that has an amino acid sequence that is different to the reference protein. In a further embodiment, the invention provides a method for designing a novel protein having a predetermined functional property and involves obtaining a sequence of a reference protein having the predetermined functional property, identifying a plurality of mutation tolerant positions in the reference protein by comparing its amino acid sequence to the amino acid sequences of a plurality of related proteins having the same functional property, and substituting at least one amino acid present at the mutation tolerant positions to produce a novel protein that has an amino acid sequence that is different to the reference protein.

In the event that the crystal structure of a protein is known, the amino acids that are implicated in the activity of the reference protein can be predicted. If only the primary structure of the reference protein is known a three-dimensional structure can be modeled using computational protein modeling software. Accordingly, in some embodiments, the method comprises obtaining a three dimensional model of the reference protein, identifying a plurality mutation tolerant positions in the reference protein by determining amino acids not involved in the active site, and substituting at least one amino acid present at the mutation tolerant position to produce a novel protein that has the known predetermined activity. In some embodiment, the reference sequence is compared to a plurality of related proteins having the same biological activity by aligning the reference protein and related protein amino acids sequences to make a sequence alignment. In a preferred embodiment, the reference protein and the related proteins have at least about 30% sequence identity. In another embodiment, the amino acid sequence of a variable region of the reference protein is compared to the variable region of the related proteins and the substitutable positions (i.e. mutation tolerant positions) is in the variable region.

In some embodiments, identifying a plurality mutation tolerant positions may be identified in a reference protein having a known biological activity by comparing its amino acid sequence (or structure) to the amino acid sequences (or structure) of a plurality of related proteins having the same biological activity. Aspects of the invention also provides a method for designing a novel protein having a predetermined biological activity, involving obtaining a sequence of a reference protein having the predetermined biological activity, screening a plurality a possible variants in silico to produce a rank ordered list of variants and substituting the amino acids present at the highest ranked mutation tolerant positions to produce a first library of proteins variants having an amino acid sequence that is different to the reference protein. In further embodiment, nucleic acid molecules that encode at least 10 of the protein variants are generated and expressed to produce the protein variants. The first library of novel proteins is then screened for the predetermined functional property and a first set of novel proteins having the least homology to the reference protein and the highest predetermined biological activity is selected. In a further embodiment, the first set of novel proteins is screened in silico to produce a rank ordered list of variants, the amino acids present at the highest ranked mutation tolerant positions can be substituted to produce a library of proteins having an amino acid sequence that is different to the reference protein and to the first library of protein variants, nucleic acid molecules that encodes at least 10 of the protein variants are generated and expressed to produce the protein variants. Protein variants may be screened for the predetermined functional property and a second set of novel proteins having the least homology to the reference protein and the highest predetermined biological activity is selected. The process can be reiterated to select a third set of novel proteins having the least homology to the reference protein and the highest predetermined biological activity. For example, the novel protein has less than about 95%, 90%, 80%, 70%, 60% homology to the reference protein sequence. The least homology is, for example, no less than 90%, 80%, 70%. One should appreciate that the novel protein can have similar, higher or lower biological activity compared to the reference protein. For example, the novel protein has at least about 95%, 90%, 80% of the reference protein functional property or biological activity.

Aspects of the invention are useful for designing novel protein having similar structural properties than reference proteins for example similar thermostability, solubility or expression level and substitution in a mutation tolerant position does not reduce the protein functional property, biological activity, stability, solubility, and expression level. In some embodiments, the mutation tolerant position correspond to solvent-accessible amino acids, amino-acids at least a pre-determined distance from the active site, amino acids not involved in stabilizing secondary, tertiary or quaternary protein structure.

In some aspects, the invention provides method of designing a library of variant proteins, the method comprising identifying a reference protein that exhibits a biological activity, determining at least one qualitative feature of the reference protein; the qualitative feature being divisible into at least a first and a further constrained second gradient level, applying to the reference protein a design algorithm to generate a plurality of variant protein sequences that comprise the qualitative feature corresponding to the first gradient level, generating a plurality of nucleic acid molecules having predefined sequences encoding the plurality of variant proteins, expressing the nucleic acid molecules to produce the variant proteins and screening the variant proteins for biological activity to identify a functional variant protein exhibiting the biological activity. The steps of applying, generating and expressing may be repeated with the functional variant protein as the reference protein and using a design algorithm to generate a second plurality of variant protein sequences that comprise the qualitative feature corresponding to the second gradient level and screening the second plurality of variant protein sequences to identify a functional variant protein exhibit the biological activity and have the qualitative feature corresponding to the second gradient level. In a further embodiment, the applying, generating expressing and screening steps may be repeated with further constrained levels of the qualitative feature until a functional variant protein with target level of the qualitative feature is determined.

Other aspects of the invention relate to methods for designing and assembling nucleic acid or protein variant libraries containing a plurality of predetermined nucleic acid or amino acid sequences. In some embodiments, the invention provides methods for designing and assembling libraries that express a plurality of polypeptides containing predetermined amino acid sequence variants. Aspects of the invention include methods for designing and assembling polypeptide expression libraries that are enriched for polypeptide sequence variants having one or more desirable traits. Aspects of the invention provide methods for filtering nucleic acid sequences to exclude those that express polypeptides having one or more unwanted traits (e.g., poor solubility, immunogenicity, instability, etc., or any combination thereof).

Aspects of the invention also provide methods for assembling an expression library that is representative of predetermined sequences of interest. Accordingly, aspects of the invention also provide expression libraries (e.g., filtered expression libraries), methods of using expression libraries to identify polypeptides having functional or structural properties of interest, and isolated polypeptides and nucleic acids encoding them.

Aspects of the invention are useful for generating pools of different polypeptides containing predetermined amino acid sequence variations. Certain aspects of the invention are useful for generating pools of candidate polypeptides that exclude variants having unwanted biophysical and biological traits. By excluding unwanted traits, a library of the invention may include a higher proportion of potentially useful polypeptide variants. As a result, a candidate polypeptide identified in a screen or selection may be more likely to have appropriate in vivo traits in addition to a functional or structural property of interest.

According to aspects of the invention, a relatively smaller expression library may be generated when unwanted polypeptide variants are excluded. For example, the number of clones required to represent all variants in a library will be smaller if the library is designed to exclude a subset of possible variants that are predicted to have unwanted traits. As a result, a relatively smaller library may be used to screen or select for a function or structure of interest when a subset of sequences is excluded from the library. Alternatively, a library of a predetermined size may be used to represent a higher number of potentially interesting polypeptide variants when unwanted variants are excluded. Accordingly, by excluding amino acid sequences that are predicted to have one or more unwanted traits, aspects of the invention may be useful to generate libraries that represent i) a higher number of potentially useful amino acid substitutions at a predetermined number of positions, or ii) potentially useful amino acid substitutions at more positions, or a combination thereof, relative to libraries that are not filtered.

Accordingly, aspects of the invention may involve imposing certain biophysical and/or biological constraints on the identity of the polypeptides that are expressed by a library. This approach can save time and cost in a screen or selection when compared to a typical approach that involves selecting a population of proteins for a required function (e.g., binding or catalytic activity) and subsequently evaluating each selected protein for stability, solubility, and/or ease of production. When a therapeutic protein is developed, immunogenicity often is evaluated last, and often after a large investment of resources in a candidate protein. In contrast, aspects of the invention may involve pre-filtering libraries for stability, solubility, and/or lack of immunogenicity in the early stages of therapeutic development (e.g., during a library design stage). As a consequence, libraries entering selection may be enriched for stable, soluble, and/or non-immunogenic sequences, leading to a lower incidence of selected proteins having properties that are unacceptable for production, storage, and/or therapeutic administration to a patient.

In some aspect of the invention, a library design may include features that are identified form the results of a prior functional or structural screen (e.g., of a library of expressed polypeptides. In some embodiments a first library may be screened using a functional screen for a favorable biological or biophysical trait. A nucleic acid library may be expressed and the expressed polypeptides may be evaluated for the favorable biological or biophysical trait for instance, for stability, solubility, and/or lack of immunogenicity. As an example, to screen polypeptide components of the library for stability, each expressed polypeptide can be tested to assess if the polypeptide retains it native structure at increased temperature or at increased concentration of chaotropic reagent. The polypeptide components of the library can be assessed for any desired property. For instance, the immunogenicity of components of the library can be assessed by expressing the polypeptides and contacting immune cells with the polypeptide wherein the assay comprises a specific readout for the an immunogenic response. In some embodiments the functional screen is used as a final screen, i.e. resulting in a library with components with desired properties. In some embodiments the components identified in the functional screens are analyzed to redesign or further design the library resulting in pre-filtered library for a specific characteristic. For instance, polypeptides classified as stable, may all comprise a specific amino acid at a specific position (e.g. an alanine at position 64). A library optimized (or pre-filtered) for increased stability can than be redesigned to comprise the specific amino acids, amino acid patterns or functional characteristics (e.g. a an alpha-helix structure) as a non-variable component (e.g. all library components of the improved library will have an alanine at position 64).

In some embodiments multiple screening events can be performed. The polypeptides identified in an initial screen can be analyzed to redesign the library and the redesigned library can be synthesized and screened again. The additional screen may have the same cut-off parameters as the first screen. For instance, the expressed polypeptides can be required to have the same stability as in the first screen. In some embodiments, the additional screen may have more stringent cut-off parameters. For instance, if in an initial screen all polypeptides stable in 3M chaotropic reagent are selected, in the additional screen only polypeptides stable in 4 M chaotropic reagent are selected. The polypeptides identified in the additional screen can again be analyzed to identify specific amino acids or patterns of amino acids or other characteristics that confer the desired biological or biophysical traits to the library components. For instance, in the first screen an alanine at position 64 may be identified, while in an additional screen the requirement for a N-terminal alpha-helix may be identified. The library can subsequently be redesigned to comprise the amino acids, amino acid patterns or other characteristics that confer a specific type or level of a biological or biophysical trait. Further rounds of screening can be performed to arrive at a library with desired properties. The library can thus be improved iteratively by cycling through multiple rounds of design and functional screening.

In some embodiments the additional screen selects polypeptides for a different biological or biophysical trait than the initial screen. For instance, the initial screen may identify polypeptides with increased stability while the additional screen may identify polypeptides with decreased immunogenicity, thereby optimizing the library for multiple desired traits. In some embodiments information obtained from initial screens for one or more desired traits can be used to pre-filter a library for a next round of design and, optionally, screening. In some embodiments the library is redesigned after each screen. In some embodiments the library is redesigned after one round of multiple screens for different biological and biophysical traits. For instance, a number of functional screens can be performed to optimize a library for multiple characteristics (e.g. one screen for stability, a second screen for solubility and a third screen for immunogenicity). Polypeptides identified in one or more of the screens can than be analyzed to redesign a library that is optimized for these multiple traits. In some embodiments the library is redesigned and resynthesized after each round of screening for one or multiple characteristics. In some embodiments the library is subjected to multiple rounds of screening before the library is redesigned and resynthesized.

According to the invention, the information from a screen for a functional or structural trait may be used at the design stage of a subsequent library. In some embodiments, a scaffold may be updated to include features from a functional screen. In some embodiments, information from an initial screen may be used to filter out theoretical variants before they are assembled. For example, certain motifs or sequences determined to be undesired in a first screen may be pre-filtered during a second screen.

In some embodiments, the invention may include methods of analyzing and/or filtering sequences that are predicted or known to confer one or more unwanted traits. In some embodiments, the invention may include methods of designing and/or assembling a library of nucleic acids having predetermined sequence differences (e.g., that encode a predetermined pool of polypeptides having predetermined amino acid changes at predetermined positions). In some embodiments, the identity of different polypeptides that are expressed by a library may be predetermined by analyzing possible amino acid sequence variants and excluding those that are predicted or known to confer one or more unwanted traits.

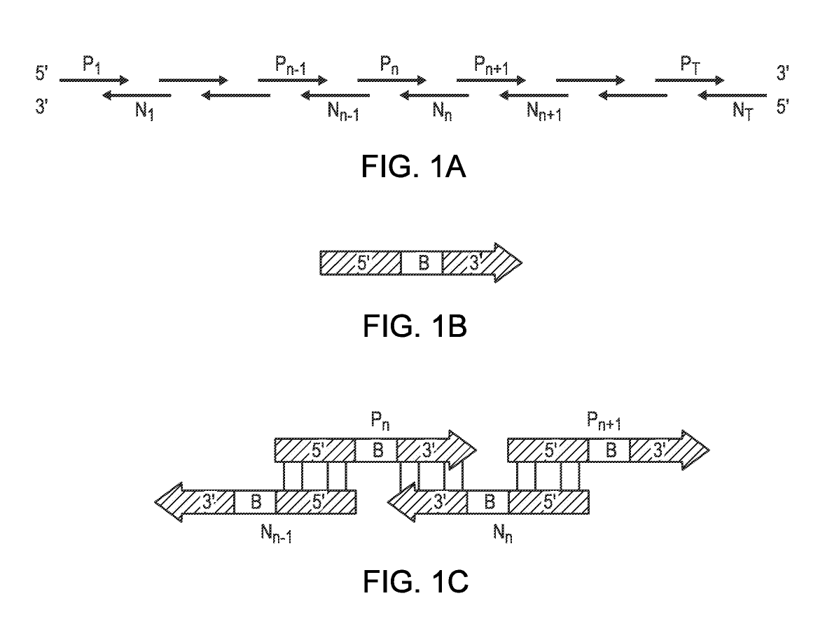

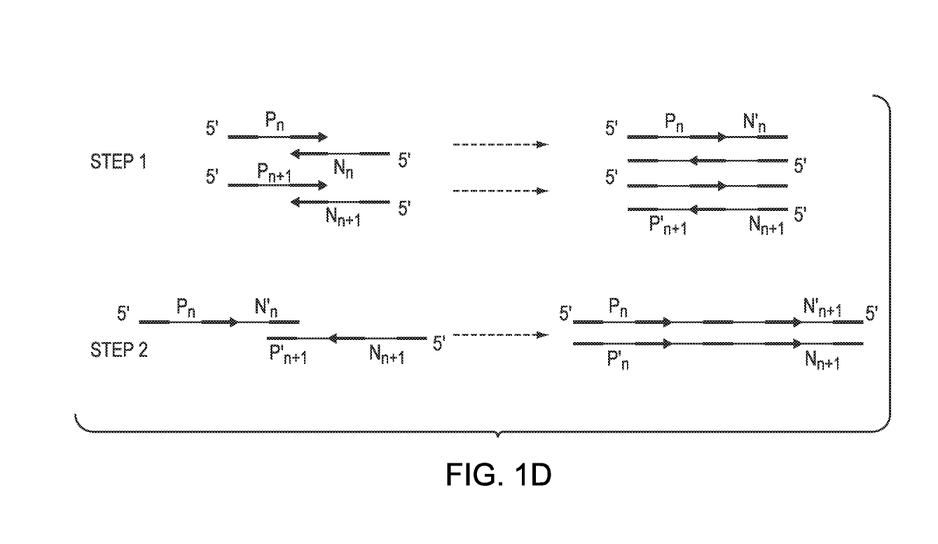

According to aspects of the invention, a library containing a large number of different nucleic acids having defined sequences may be assembled using any suitable in vitro and/or in vivo nucleic acid assembly procedure that allows a plurality of specific sequences to be assembled while excluding other specific sequences. According to aspects of the invention, a library may be assembled in a process that involves assembling a plurality of nucleic acids (e.g., polynucleotides, oligonucleotides, etc.) to form a longer nucleic acid product. A library may contain nucleic acids that include identical (non-variant) regions and regions of sequence variation. Accordingly, certain nucleic acids being assembled may correspond to the non-variant sequence regions. Other nucleic acids being assembled may correspond to one of several predetermined sequence variants in a predetermined region of sequence variation. Non-limiting examples of assembly reactions are described herein and illustrated in FIGS. 1-4. It should be appreciated that one or more of the nucleic acids illustrated in FIGS. 1-4 may be a mixture of nucleic acids that contain one or more identical shared sequence regions, for example the 5′ and 3′ regions that are designed to overlap with adjacent nucleic acids during the assembly procedure, and one or more unique sequence regions, for example one or more regions corresponding to a single predetermined sequence variant. It should be appreciated that aspects of the invention may be automated (e.g., using computer-implemented analyses, assemblies, screens, selections, etc.).

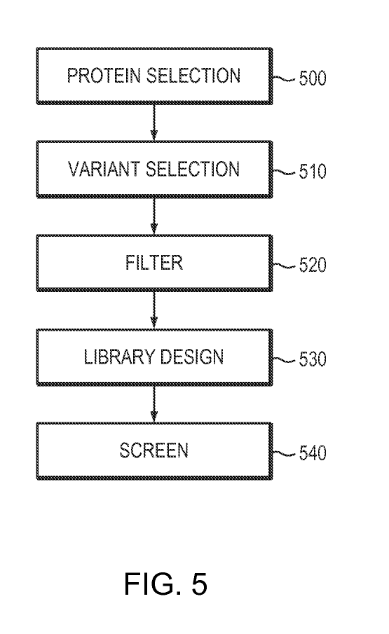

FIG. 5 illustrates one aspect of a process of designing a library that expresses polypeptide variants having predetermined thresholds for one or more biophysical and/or biological traits. Initially, in act 500, a protein that may be used as a scaffold for the library is selected. A scaffold protein will have a selected number or amino acids or functional and/or structural elements that are fixed. For instance, a specific polypeptide scaffold library may have a lysine at position 4 and a DNA binding domain at its N-terminus, but all other positions may be varied during the design, screening and synthesis stages. In act 510, positions at which amino acids may be changed are determined. In some embodiments, a corresponding list of all potential amino acid sequence variants may be identified. This list may be referred to as a theoretical library of polypeptide sequences that can be analyzed and filtered to exclude unwanted sequences in act 520. In act 530, a library is designed and assembled to express all of the filtered polypeptide sequence variants or a fraction thereof. In act 540, a screen, selection, or other analysis is performed to identify one or more polypeptides in the library that have one or more structural or functional properties of interest. It should be appreciated that one or more of these acts may be omitted in certain embodiments of the invention. It also should be appreciated that one or more of these acts may be automated (e.g., computer-implemented).

In act 500, a polypeptide scaffold is selected. A library may be designed to express any type of polypeptide (e.g., linear polypeptides, constrained polypeptides, and variants thereof). A polypeptide scaffold may be based on, but is not limited to, one of the following peptides: cysteine-rich small proteins (e.g., toxins, extracellular domains of receptor proteins, A-domains, etc.), Zinc fingers, immunoglobulin-like domains (including, for example, the tenth human fibronectin type III domain and other fibronectin type III domains), lipocalins, lectin domains (including, for example, C-type lectin domain), ankyrins, human serum proteins (including, for example, human serum albumin), antibodies and antibody fragments (including, for example, single-chain antibodies, Fab fragments, single-domain (VH or VL) antibodies, camel antibody domains, humanized camel antibody domains), enzymes (including, for example, glucose isomerase, cellulase, hemicellulase, glucoamylase, alpha amylase, subtilisin, lipases, dehydrogenases, etc.), DNA-binding proteins (including, for example, the lac repressor, tip repressor, tet repressor, CAP activator, etc.), cytokines (including, for example, IL-1, IL-4, IL-8, etc.), hormones (including, for example, insulin, growth hormone, etc.), other suitable proteins, or combinations thereof.

General features that are useful for a scaffold polypeptide to have may include one or more of the following non-limiting features: a known structure; high stability and solubility; low immunogenicity; ease of expression in microbial system and ease of purification; a combination of residues that provide a well-defined, stable folded structure, and residues that can be mutated or randomized without destroying the overall fold (such ‘randomizable’ residues may be solvent-exposed or may not be involved in secondary structure or may not pack against other residues in the structure—when comparing sequences of homologous proteins, there is more variation between residues between residues in ‘randomizable’ positions than between residues critical for structure); positions/residues that are known to be associated with a particular structural motif, these could be conserved residues or residues that have been identified by structural analysis or mutagenesis to be important for preserving a structural scaffold; a scaffold of a protein that performs a function related to the desired function; independently folded domains of multi-domain proteins; and/or a monomeric state (associates with no other proteins, or only minimal number of other proteins that will either not be present during application or that are important for the function that is being engineered).

In some embodiments libraries of scaffold polypeptides are polypeptides with a specific biological function. Examples of biological functions are binding, inhibiting a biological process, catalyzing a specific reaction, etc. An example of a library of scaffold polypeptides with a specific biological function are polypeptides that can bind to a linear polypeptide and polypeptides that can bind to a phosphotyrosine.

Scaffolds of polypeptides that bind linear peptides can be based on proteins that are evolved to bind linear polypeptides. These proteins include major histocompatibility complex proteins (MHC I and MHC II), peptide transporter proteins, chaperones, proteases, and multi-domain proteins comprising peptide-binding domains such as poly(A)-binding protein, SH2 domains, SH3 domains, PDZ domains, and WW domains.

Major histocompatibility complex proteins display peptides of 9-12 amino acids on the surface of antigen-presenting cells, where the MHC-peptide complex can be recognized and bound by T-cell receptor. Humans have several hundred different MHC alleles, which vary in their specificity and affinity for specific peptides. MHC polypeptide scaffolds are designed based on the analysis of theses alleles. Peptide transporter proteins bind to linear peptides of 2-18 amino-acid residues, and bury at least a part of the peptide in their core. The transporter-peptide complex can subsequently be translocated across the membrane with the help of additional transport complex components. One example of a peptide transporter is the oligopeptide permease (Opp) family, with different members of the family recognizing peptides of different lengths and sequences with nanomolar to micromolar affinity. One member of the family, the Opp protein of Lactococcus lactis (OppAL1) can bind and transport peptides of up to 18 residues and longer. Polypeptide scaffolds are designed based on the analysis of the peptide binding properties, including the core region, of OppAl1 and other peptide transport proteins. Proteases cleave polypeptides, and differ widely by their degree of substrate specificity. Inactive mutants have been constructed that bind polypeptides, but do not cleave them. These mutant proteases are therefore particularly suited as scaffold polypeptides for polypeptides with peptide binding properties. The poly(A)-binding protein (PABC) has a C-terminal domain of interacts with translational factors in a random-coil configuration. The peptide motif that binds to PABC comprises 12-15 amino-acid residues and is in a formation resembling random-coil when bound to PABC. The peptide binding domain of PABC of various species can be analyzed to identify residues essential to peptide binding. Scaffold polypeptide for libraries of peptide binding polypeptides are designed based on these principles.

Scaffolds of polypeptides that bind phosphotyrosines can be based on proteins that are evolved to bind and/or process phosphotyrosines. Phosphotyrosine binding and processing proteins include proteins with phosphotyrosine-binding (PTB) domains, protein tyrosine phosphatases (PTPs), and mitogen-activated protein kinase (MAPK) phosphatases (MKPs). Phosphotyrosine-binding (PTB) domains are naturally occurring phosphotyrosine binding modules. The protein structure generally falls under the pleckstrin homology (PH) superfold. The peptides are recognized in general according to the motif N-P-X-(phosphoY/Y/F) whit the peptide binding as a type I beta turn. Examples of mammalian PTBs include Shc, Sck, X11, Doc-2, and p96, while drosophila PTBs include Dab and Numb. There are at least 50 PTB domains known from at least 46 proteins, with many structures elucidated by NMR or crystal structures, for instance Shc, X11, IRS-1, Talin, Dab1/2, Numb, SNT, Dok1/5, Radixin, and tensin1. Proteins with PTBs are analyzed to design a scaffold polypeptide for phosphotyrsosine binding. Extra weight will be given to proteins that bind phosphotyrosine peptides in a phosphotyrosine dependent manner. Examples of such proteins include Shc-like PTBs, and IRS-like PTBs (which include IRS, Dok, and SNT) and proteins including the C2 domain of PKCδ and possibly PKCθ. Protein tyrosine phosphatases (PTPs) often play a critical role in cellular regulation by dephosphorylating tyrosines of signaling molecules. PTPs include both receptor-like PTPs and non-transmembrane PTPs. Some examples of PTPs are SHP-2 (PTPN11), PTP-1B (PTPN1), TCPTP (PTPN2), PEP (PTPN22), SHP-1 (PTPN6), PTP-PEST (PTPN12), PTP-MEG2 (PTPN9), STEP (PTPN5), and HePTP (PTPN7). While PTPs process phopshotyrosine peptides, the phosphatase activity can be inactivated resulting in a polypeptide that can bind phosphotyrosines but can not process them. The PTP active site generally contains the motif HC(X₅)R, and may additionally contain a WPD motif. The dephosphorylation function can be inactivated by introducing one or more mutations in the active-site. For example, the essential C and/or R (such as C-S), and/or the invariant D (such as D-A), or combinations thereof (such as C-S/D-A or D-A/Q-A) can be mutated to result in an inactive phosphatase activity. Scaffold polypeptides are designed based on these inactivated PTPs. Mitogen-activated protein kinase (MAPK) phosphatases (MKPs) are related to PTPs and can dephosphorylate both phosphothreonine and phosphotyrosine residues. MPKs are found in various mammalian pathways, including ERK, JNK (MAPK8), p38 (MAPK14). The active site of these proteins is mutated to result in a polypeptide scaffold and this polypeptide scaffold san subsequently be used as a scaffold for a library of phosphotyrosine binding polypeptides.

However, in some embodiments, a library may be designed to express random polypeptides that are not based on any defined structural scaffold.

In act 510, residues that may be changed in the library may be identified.

General features that may be used for selecting one or more residues to be varied in the library may include one or more of the following non-limiting features: residues in a binding domain (for example a receptor binding domain, a ligand binding domain or a substrate binding domain), in particular residues in contact with, or adjacent to a bound ligand; residues in a catalytic domain, in particular residues in, or immediately adjacent to, an active site; adjacent residues, for example residues that on the surface of a protein that may be modified to make an artificial antibody; surface residues; buried residues, for example proteins can be stabilized by re-engineering their core; residues that are thought to, or known to, tolerate changes without affecting the structure of the scaffold; residues that vary between homologous proteins; and/or residues that have been shown to affect function.

If there is a long list of residues that can be changed, a hierarchy to select the preferred subset to be altered may be established. The hierarchy depends on the application. One potential hierarchy is the following:
(1) avoid destabilization of the protein;
(2) for therapeutic proteins, minimize the number of residues to be randomized in order to minimize the risk of immunogenicity;
(3) provide a large enough variability in the shape of a possible target-binding surface or in the chemistry of a catalytic active site to maximize the chance of selecting a variant with new function;
(4) limit the number of randomized positions to positions that may affect each other; aim to sample every possible permutation of residue on those positions; and
(5) limit the number and nature of replacements at each position based on their predicted effect on the function.

Once positions to be varied are identified, a theoretical library may be determined that includes all combinations of possible amino acid variants at those positions. In some embodiments, all natural amino acid variants are considered (e.g., the 20 amino acids that are present in most natural proteins or polypeptides). In some embodiments, non-natural amino acids also may be considered. However, in some embodiments a first library may be designed to include a subset of variants.

In act 520, the theoretical library may be filtered to identify and/or exclude sequence variants that are known or expected to confer one or more unwanted traits. One or more filtering steps may be implemented to identify and/or exclude one or more different traits that may be unwanted. Filtering may be based on predicted properties of amino acid sequences, known properties of amino acid sequences, or combinations thereof. It should be appreciated that the trait(s) selected to be excluded may depend on the application that is being screened for. For example different types of predictions may be relevant to different applications. In some embodiments, library filtering based on predicted immunogenicity would be irrelevant if the library is to be screened for better industrial enzymes. In some embodiments, the largest number of filters that are relevant for a particular application may be incorporated in filtering act 520.

Filter parameters that may be useful to select sequence variants that are known or expected to confer one or more unwanted traits may include one or more of the following non-limiting parameters: a) immunogenicity (T-cell epitopes may be removed—algorithms for predicting T-cell epitopes may be used—other known or predicted epitopes also may be removed—non-limiting examples for reducing the immunogenicity of a protein are reported in US Patent Publications US20060025573 and US20040082039, the disclosures of which are hereby incorporated by reference); b) other immunogenicity-related properties, including aggregation, binding to receptors on antigen-presenting cells, proteosome cleavage, transport of cleavage product by TAP, the transporter associated with antigen processing; c) other factors that determine immunogenicity including factors reported in US Patent Publications US20040203100, US20060073563, US 20060014248, US20050079183 and US20050214857; U.S. Pat. No. 6,929,939 and WO2003104803, the disclosures of which are hereby incorporated by reference; d) solubility; for instance including calculating the predicted pI of a sequence and excluding the sequence if the pI is within 0.5 pH units, within 1 pH unit, within 2 pH units, within 3 pH units, within 4 pH units, or within 5 pH units, of the pH at which the polypeptide may be expressed, purified, stored and/or used; e) stability; for instance including structure based methods, molecular modeling methods and other computer based methods (see e.g. US Patent Publications US20060073563 and US20060014248); f) the presence of sequences that are undesirable, for instance including protease sensitive sequences, toxic sequences and sequences that are known to interact with unwanted targets; g) the exclusion of Cys residues that are not close enough to form disulfide bonds in a folded structure based on the known structure of the scaffold; h) the exclusion of excessive numbers of Trp residues, in some embodiments 2, 3, 4, or more Trp residues can be excluded; and i) the exclusion of chemically active sequences of amino acids, for instance asparagine and glutamine deaminate more readily when followed by a glycine.

Accordingly, a final library of filtered peptide products to be synthesized may be determined. It should be appreciated that different filtering parameters may be varied in order to increase or decrease the stringency of the filtering process.

In some embodiments, a filtering process may proceed according to the following steps. First, a list of more than 100 related protein sequences may be generated based on available information of a scaffold structure and function. Second, each sequence may be subjected to an automatic calculation to evaluate the property of choice; sequences with values below the cutoff will be eliminated from the list. This step may be repeated for each property under examination. Third, selected protein sequences may be reverse-transcribed into DNA sequences. Each DNA sequence may be optimized for codon usage, secondary structure formation, presence of restriction sites, etc., without changing the protein sequence. Optimized DNA sequences on the list then may be assembled using any appropriate assembly method.

To validate the improvement of properties due to a pre-filtering strategy, parallel DNA libraries may be generated initially with and without the theoretical pre-filtering step. Randomly selected members of pre-filtered and unfiltered libraries may then be translated into protein and tested for the property under investigation. In addition, in-vitro selections may be performed under identical conditions for pre-filtered and unfiltered libraries, and the properties of the selected proteins from each may be compared.

In some embodiments, libraries may be filtered for high solubility. For example, a simple method of predicting protein solubility based on its sequence is through the calculation of its isoelectric point (pI), the pH where the protein has no net charge. Numerous well-established algorithms are available for calculating the pH of a given sequence (e.g., http://www.scripps.edu/˜cdputnam/protcalc.html, http://www.embl-heidelberg.de/cgi/pi-wrapper.pl). In some embodiments, a protein is predicted to be soluble if its pH is significantly higher or lower than the pH (e.g., by 0.5 pH units or more) of the buffer employed to purify and/or use the protein.

Other possible measures of solubility include overall hydrophobicity of the protein, which can be either the proportion of amino-acid residues in the protein that are apolar, or the proportion of residues predicted to be accessible to the solvent that are apolar. Alternatively, only the number of tryptophan residues can be limited, or cysteine residues can be prohibited from randomized positions.

In some embodiments, representative members of libraries and selected proteins can be evaluated for solubility by comparing their expression level, the concentration beyond which they aggregate, or the proportion of protein sample at a set concentration that aggregates when incubated at a set temperature.

In some embodiments, libraries may be filtered for low immunogenicity. The immunogenicity of a protein can be predicted computationally by breaking down the protein into a series of overlapping peptides, then evaluating the fit of each resulting peptide to the peptide-binding site of an MHC type II molecule (Chirino et al, Drug Discovery Today (2004), 83; e.g., Jones et al (2004), J. Interferon Cytokine Res 24, 560). In certain embodiments, peptide sequences can be compared to databases of peptide sequences known to bind such MHC II molecules, or known to stimulate T-cells (Novozymes).

Representative members of libraries and selected proteins can be evaluated for immunogenicity by expressing and purifying each protein in a microbial system, then testing their ability to stimulate T-cells from diverse human donors. Individual peptides that make up the protein or pools of such peptides can also be tested for their ability to stimulate T-cells. In some embodiments, proteins can be evaluated by injecting them into transgenic mice that express the human version of the scaffold the proteins are based on.

In some embodiments, libraries may be filtered for high stability. In some embodiments, in order to predict the stability of each protein, its three-dimensional structure can be simulated computationally and evaluated for favorable and unfavorable interactions (Chirino et al, Drug Discovery Today (2004), 83; e.g., Luo et al (2002) Protein Sci. 11, 1218). In certain embodiments, the simulated structure could be compared to the known structure of the scaffold it is based on, or to known structures of proteins that are homologous to the scaffold. In some embodiments, structures that are more similar to existing protein structures are predicted to be more stable. In some embodiments, the effect of a mutation on scaffold stability can be studied experimentally before embarking on library construction. For example, each position in the scaffold can be separately mutated to all possible amino acids, and the resulting mutant proteins can be expressed and evaluated for stability, solubility, or both. Libraries based on that scaffold can then be designed to avoid mutations that have been shown to destabilize the scaffold.

Representative members of libraries and selected proteins can be evaluated for stability by comparing their expression level, melting temperature, concentration of urea or guanidine required to denature them, or the proportion of each protein sample at a set concentration that aggregates when incubated at an elevated temperature.

In act 530, a library of filtered sequences may be obtained (e.g., assembled as described herein). The library may be cloned into any suitable vector (e.g., any suitable expression vector) in any suitable organism. Any suitable vector may be used, as the invention is not so limited. For example, a vector may be a plasmid, a bacterial vector, a viral vector, a phage vector, an insect vector, a yeast vector, a mammalian vector, a BAC, a YAC, or any other suitable vector. In some embodiments, a vector may be a vector that replicates in only one type of organism (e.g., bacterial, yeast, insect, mammalian, etc.) or in only one species of organism. Some vectors may have a broad host range. Some vectors may have different functional sequences (e.g., origins or replication, selectable markers, etc.) that are functional in different organisms. These may be used to shuttle the vector (and any nucleic acid fragment(s) that are cloned into the vector) between two different types of organism (e.g., between bacteria and mammals, yeast and mammals, etc.). In some embodiments, the type of vector that is used may be determined by the type of host cell that is chosen.

It should be appreciated that a vector may encode a detectable marker such as a selectable marker (e.g., antibiotic resistance, etc.) so that transformed cells can be selectively grown and the vector can be isolated and any insert can be characterized to determine whether it contains the desired assembled nucleic acid. The insert may be characterized using any suitable technique (e.g., size analysis, restriction fragment analysis, sequencing, etc.). In some embodiments, the presence of a correctly assembly nucleic acid in a vector may be assayed by determining whether a function predicted to be encoded by the correctly assembled nucleic acid is expressed in the host cell.

In some embodiments, host cells that harbor a vector containing a nucleic acid insert may be selected for or enriched by using one or more additional detectable or selectable markers that are only functional if a correct (e.g., designed) terminal nucleic acid fragments is cloned into the vector.

Accordingly, a host cell should have an appropriate phenotype to allow selection for one or more drug resistance markers encoded on a vector (or to allow detection of one or more detectable markers encoded on a vector). However, any suitable host cell type may be used (e.g., prokaryotic, eukaryotic, bacterial, yeast, insect, mammalian, etc.). In some embodiments, the type of host cell may be determined by the type of vector that is chosen. A host cell may be modified to have increased activity of one or more ligation and/or recombination functions. In some embodiments, a host cell may be selected on the basis of a high ligation and/or recombination activity. In some embodiments, a host cell may be modified to express (e.g., from the genome or a plasmid expression system) one or more ligase and/or recombinase enzymes.

In act 540, proteins expressed by the filtered library may be screened or selected for one or more functions or structures of interest. It should be appreciated that expression libraries of the invention may be nucleic-acid/polypeptide libraries in which each nucleic acid molecule is physically associated with the polypeptide it encodes. In some embodiments, an expression library may be a screening library. An example of a screening library may be one where the physical association between the nucleic acid and the encoded polypeptide is provided by a well (e.g., in a 96-well plate). In some embodiments, an expression library may be a display library. Examples of display libraries include those generated by phage, bacterial, yeast, mRNA, or ribosome display, where each nucleic acid and corresponding polypeptide are part of the same physical particle (e.g., a bacteriophage, a bacterium, a yeast cell, covalent mRNA-polypeptide fusion, or non-covalent mRNA/ribosome/polypeptide complex).

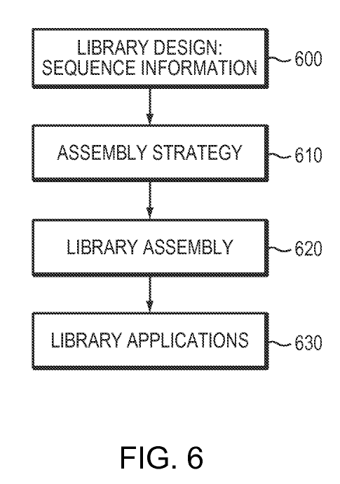

It should be appreciated that preferred methods of assembling a nucleic acid library are methods that can be used to effectively assemble a large number of defined sequence variants at predetermined positions of interest while specifically excluding other sequence variants at those positions. FIG. 6 illustrates an embodiment of a library assembly process of the invention. In act 600, sequence information is obtained defining the sequences that are to be included in the library. In act 610, an assembly strategy is formulated. In act 620, starting nucleic acids are obtained. In act 630, the starting nucleic acids are assembled to form the library. In some embodiments, the library may be used to screen or select for polypeptides having one or more properties of interest. In some embodiments, the library may be sent or shipped to a customer. In some embodiments, the library may be stored and used to generate a nucleic acid sequence library that contains a plurality of predetermined sequence variants. It should be appreciated that one or more of these acts may be omitted in certain embodiments of the invention. It should be appreciated that one or more of these acts may be automated (e.g., computer-implemented).

Initially, in act 600, information defining the specific nucleic acid sequences to be included in the library may be obtained from any source. In some embodiments, nucleic acid sequence variants to be included in a library may be those that encode polypeptide sequences that were identified in a filtering process of the invention. In some embodiments, a list of different polypeptide variants to be encoded by a library may be designed or obtained (e.g., in the form of a customer order or request). The different nucleic acid sequences to be assembled may be determined based on the identity of the polypeptide sequences to be included in a library. It should be appreciated that different nucleic acid sequences may encode the same polypeptide due to the degeneracy of the genetic code. In some embodiments, the sequence of a nucleic acid selected to code for a defined polypeptide variant may be determined based on any suitable parameter, including, for example, the codon bias in the host organism used for the library, the synthesis strategy, the relative ease of assembling certain sequences (e.g., sequences may be selected to avoid direct or inverted sequence repeats, sequences that stabilize one or more secondary structures, sequences with high GC or AT content, etc.), or any combination thereof. For example, when choosing codons for each amino acid, consideration may be given to one or more of the following factors: i) using codons that correspond to the codon bias in the organism in which the target nucleic acid may be expressed, ii) avoiding excessively high or low GC or AT contents in the target nucleic acid (for example, above about 60% or below about 40%; e.g., greater than about 65%, 70%, 75%, 80%, 85%, or 90%; or less than 35%, 30%, 25%, 20%, 15%, or 10%), iii) avoiding sequence features that may interfere with the assembly procedure (e.g., the presence of repeat sequences or stem loop structures), and iv) using codons for each amino acid such that the expression levels of some or all of the proteins in the library are normalized, for example if some desired sequences are anticipated to express less than others, it may be desirable to purposely decrease the expression level of the others, so expression bias does not affect the assay result. However, these factors may be ignored in some embodiments as the invention is not limited in this respect. In some embodiments, a customer order may include a specific list of defined nucleic acid sequences to be included in a library (e.g., for a library of defined DNA sequences, a library designed to express defined RNA sequences, etc.). A polypeptide or nucleic sequence order from a customer may be received in any suitable form (e.g., electronically, on a paper copy, etc.).

In act 610, the sequence information may be analyzed to determine an assembly strategy. This may involve determining whether the library may be assembled in a single reaction or if several intermediate fragments may be assembled separately and then combined in one or more additional rounds of assembly to generate the target nucleic acid library. Once the overall assembly strategy has been determined, input nucleic acids (e.g., oligonucleotides) for assembling the one or more nucleic acid fragments may be designed. The sizes and numbers of the input nucleic acids may be based in part on the type of assembly reaction (e.g., the type of polymerase-based assembly, ligase-based assembly, chemical assembly, or combination thereof) that is being used for each fragment. The input nucleic acids also may be designed to avoid 5′ and/or 3′ regions that may cross-react incorrectly and be assembled to produce undesired nucleic acid fragments. Other structural and/or sequence factors also may be considered when designing the input nucleic acids. In certain embodiments, some of the input nucleic acids may be designed to incorporate one or more specific sequences (e.g., primer binding sequences, restriction enzyme sites, etc.) at one or both ends of the assembled nucleic acid fragment. In other embodiments these specific sequences may be at positions within the nucleic acid fragment.

In some embodiments, information developed during the design phase may be used to determine an appropriate synthesis strategy for certain variants. For example, it may be apparent from the sequence analysis and the assembly design that certain sequences may be poorly assembled and therefore under-represented in an assembled library. In some embodiments, these sequences may be assembled separately. In some embodiments, certain sequences may be identified for a user (e.g., a customer) as likely to be under-represented in a library or absent from the library.

In some embodiments, certain input nucleic acids may include one or more variant regions that encode one of several different predetermined amino acid sequences that are part of the library. In some embodiments, an input nucleic acid may be designed to restrict the variant sequences to a central region of the nucleic acid that does not overlap with adjacent 5′ and 3′ regions.

In act 620, input nucleic acids are obtained. These may be synthetic oligonucleotides that are synthesized on-site or obtained from a different site (e.g., from a commercial supplier). In some embodiments, one or more input nucleic acids may be amplification products (e.g., PCR products), restriction fragments, or other suitable nucleic acid molecules. Synthetic oligonucleotides may be synthesized using any appropriate technique as described in more detail herein. It should be appreciated that synthetic oligonucleotides often have sequence errors. Accordingly, oligonucleotide preparations may be selected or screened to remove error-containing molecules as described in more detail herein. In one embodiment oligonucleotides will be synthesized as mixtures by using random nucleotide incorporation. The oligonucleotides can later be screened for the correct sequence.

In act 630, an assembly reaction may be performed to produce a library based on the nucleic acids obtained in act 620.

In one embodiment the sequence variability designed for a library is encoded within the size of a single assembly oligonucleotide.

If sequence variability is desired in several different regions of the polypeptide, variant regions may be required in several of the different assembled oligonucleotides. In some embodiments several parallel assembly reactions may be performed to create different subsets of the desired sequences. In some embodiments the oligonucleotides may be pre-screened prior to assembly.

For each fragment, the input nucleic acids may be assembled using any appropriate assembly technique (e.g., a polymerase-based assembly, a ligase-based assembly, a chemical assembly, or any other multiplex nucleic acid assembly technique, or any combination thereof). An assembly reaction may result in the assembly of a number of different nucleic acid products in addition to the predetermined nucleic acid fragment. Accordingly, in some embodiments, an assembly reaction may be processed to remove incorrectly assembled nucleic acids (e.g., by size fractionation) and/or to enrich correctly assembled nucleic acids (e.g., by amplification, optionally followed by size fractionation). In some embodiments, correctly assembled nucleic acids may be amplified (e.g., in a PCR reaction) using primers that bind to the ends of the predetermined nucleic acid fragment. It should be appreciated that act 630 may be repeated one or more times. For example, in a first round of assembly a first plurality of input nucleic acids (e.g., oligonucleotides) may be assembled to generate a first nucleic acid fragment. In a second round of assembly, the first nucleic acid fragment may be combined with one or more additional nucleic acid fragments and used as starting material for the assembly of a larger nucleic acid fragment. In a third round of assembly, this larger fragment may be combined with yet further nucleic acids and used as starting material for the assembly of yet a larger nucleic acid. This procedure may be repeated as many times as needed for the synthesis of a target nucleic acid. Accordingly, progressively larger nucleic acids may be assembled. At each stage, nucleic acids of different sizes may be combined. At each stage, the nucleic acids being combined may have been previously assembled in a multiplex assembly reaction. However, at each stage, one or more nucleic acids being combined may have been obtained from different sources (e.g., PCR amplification of genomic DNA or cDNA, restriction digestion of a plasmid or genomic DNA, or any other suitable source).

It should be appreciated that nucleic acids generated in each cycle of assembly may contain sequence errors if they incorporated one or more input nucleic acids with sequence error(s). At some stage during the library assembly process, fidelity optimization can be performed. In one embodiment this is done by MutS. In some embodiments, variant fragments are created and processed by MutS separately. In some embodiments the variant regions of the library are evaluated by sequencing.

In certain embodiments, constant portions of a protein scaffold may be synthesized and error-corrected. In contrast, variant positions may be assembled without error correction. In some embodiments, the presence of a background of additional sequence variants may not interfere with the library as a whole if the number of unwanted sequence errors is low relative to the number of predetermined sequence variants in the library. However, in some embodiments the presence of errors within the constant regions of the scaffold may be undesirable if these sequence errors have a negative impact on the function of the predetermined sequence variants that they are associated with.

In some embodiments, assembly reactions may be performed using assembly nucleic acids that have not been amplified (e.g., assembly oligonucleotides that were synthesized and released from an array without an amplification step). In some embodiments, a plurality of non-amplified overlapping nucleic acids may be assembled to generate one variant sequence for a library. This variant fragment may be amplified. In some embodiments, this variant fragment may be amplified using one or more universal primers if the flanking assembly nucleic acids have sequences (e.g., sequences that may need to be removed) that are complementary to the universal primers.

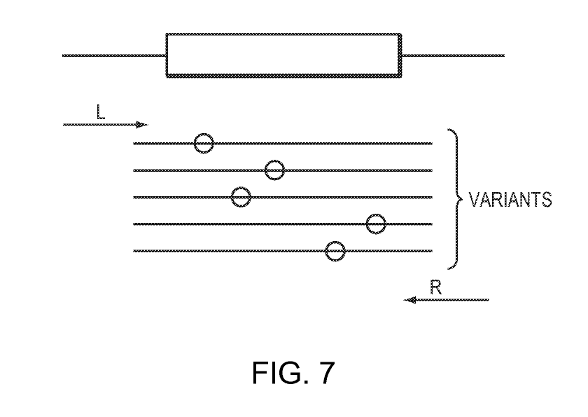

FIG. 7 illustrates an embodiment where the variant region is approximately the size of an assembly nucleic acid (e.g., an assembly oligonucleotide). In some embodiments, assembly nucleic acids designed to correspond to the same region of a target nucleic acid are designed to contain sequence variants only within their central region. These variant encoding assembly nucleic acids can be amplified by using one or more primers that bind to the non-variant 5′ and 3′ regions. Accordingly, a plurality of assembly nucleic acids (e.g., a plurality of different assembly oligonucleotides synthesized on an array), each encoding a different variant sequence, can be amplified using the same 5′ and 3′ primers (e.g., shown as L and R in FIG. 7). Accordingly, in some embodiments, these variant-encoding assembly nucleic acids are synthesized without any flanking 3′ and/or 5′ amplification sequences (e.g., without any sequences that correspond to universal primer sequences). These assembly nucleic acids can be amplified and used for assembly without removing flanking amplification regions. However, in some embodiments these variant-encoding assembly nucleic acids are not amplified and are used directly in an assembly reaction (e.g., after release from a solid support such a synthesis array). Accordingly, L and R in FIG. 7 may be adjacent assembly nucleic acids such as adjacent oligonucleotides in the assembly reaction. It should be appreciated that these adjacent oligonucleotides also may be used prior to amplification. In some embodiments, the variant-encoding assembly nucleic acids shown in FIG. 7 are designed to span a region between a 5′ fragment of a gene and a 3′ fragment of the same gene. The 5′ and 3′ fragments may be prepared using any suitable technique (e.g., by amplification, restriction enzyme cloning, etc.). Accordingly, L and R in FIG. 7 may be the 5′ and 3′ gene fragments in some embodiments. The 5′ and 3′ fragments and the variant-encoding assembly nucleic acids may be designed to include a first region of sequence overlap between the 3′ end of the 5′ fragment and the 5′ end of the assembly nucleic acids and a second region of sequence overlap between the 3′ end of the assembly nucleic acids and the 5′ end of the 3′ fragment (as illustrated in FIG. 7). Accordingly, the variant-encoding assembly nucleic acids (e.g., non-amplified) may be mixed with the 5′ and 3′ gene fragments and assembled in a polymerase-based or a ligase-based extension reaction.

Libraries the invention can be used in any method for in-vitro protein evolution, screening, or selection.

In some embodiments, a recombinase (e.g., RecA) or nucleic acid binding protein may be used to increase the fidelity of one or more assembly reactions. In some embodiments, a heat stable RecA protein may be included in one or more reagents or steps of a multiplex nucleic acid assembly reaction. A heat stable RecA protein is disclosed, for example, in Shigemori et al., 2005, Nucleic Acids Research, Vol. 33, No. 14, e126. Heat stable RecA proteins may be from one or more thermophilic organisms (e.g., Thermus thermophilus or other thermophilic organisms). Heat stable RecA proteins also may be isolated as sequence variants of one or more heat sensitive RecA proteins.

Aspects of the invention may include automating one or more acts described herein. For example, an analysis may be automated in order to generate an output automatically. Acts of the invention may be automated using, for example, a computer system.

Aspects of the invention may be used in conjunction with any suitable multiplex nucleic acid assembly procedure involving at least two nucleic acids with complementary regions (e.g., at least one pair of nucleic acids that have complementary 3′ regions). For example, library assembly may involve one or more of the multiplex nucleic acid assembly procedures described below.

### Protein Engineering Using Rational Diversity

De novo protein design methodologies have become significantly more powerful in the past decade. It is now possible to screen libraries of >10¹⁰⁰ protein sequences in silico, not by computationally checking each one, but rather by exploiting an algorithm to eliminate regions of sequence space. See Design of a Novel Globular Protein Fold with Atomic Level Accuracy, Kuhlman et al., Science, V203, p. 1344, 2003. These library sizes are staggering in comparison with experimental methods, which top out at library sizes of about 10¹² to 10¹⁵.

The caveat of in silico methods is that they rely heavily on empirical models of protein function, and thus, currently have far less than perfect accuracy. To compensate for model inaccuracies, the output of in silico models is generally a rank-ordered list of possible designs, where each design is assigned a score. One then ends up with a list of “highly likely solutions” at the top of this ordered list, some subset of which can be synthesized or mutated from wild type sequences and tested. Still, this approach has had some notable successes recently. For example, a novel 27 amino acid sequence αββ motif with a predefined backbone was designed (Dahiyat and Mayo 1997, Science 278: 82-87), a novel iron superoxide dismutase was designed (Pinto et al. 1997, Proc. Natl. Acad. Sci. USA 94: 5562-5567), a novel 93 amino acid protein fold not found in nature, “Top7” was designed (Kuhlman et al. 2003, Science 302: 1364-1368), addition of enzymatic activity (triose phosphate isomerase) into a nonenzyme scaffold (ribose binding protein) was achieved through protein design (Dwyer et al. 2003, Science 304: 1967-1971), novel sensor proteins were designed (Looger et al. 2003, Nature 423: 185-190), and a therapeutic protein variant (dominant negative TNF-alpha variant) has been designed (Steed et al 2003, Science 301: 1895-1898).

The field is becoming increasingly aware that the empirical models used to score each design may not be sufficiently good to separate the best 10 or 20 designs from the others. This was highlighted in a recent paper pointing out how some models are used to make predictions far from their optimal regimes (Jaramillo and Wodak 2005, Biophys. J 88: 156-171). Practitioners have a desire to synthesize and test more than about 10 of their in silico designs, perhaps 100 to 1000 or even 10000 proteins instead, to avoid missing possible solutions to the design problem due to only a slight error in the model.

In silico designs can be made to produce a library of constructs that can serve as a pool or plural separate species that can be tested or selected for a good candidate, or can serve as a starting places for other purposeful design iterations or for evolutionary techniques utilizing random mutagenesis. A screen or selection can be applied to the pool, and if necessary, the process (starting from design or another library expansion) can be iterated. This general strategy is referred to herein as “rational diversity” and emphasizes the importance of a mechanistic model (“rational”) in the initial library design.

Design is a necessity for what cannot be done (or cannot be done in a reasonable amount of time) by mutation or evolution. Fundamentally, this arises from the difference in library sizes for computational versus experimental screens. Natural biological evolution and derivative laboratory techniques like directed evolution have two important constraints. First, intermediates must be viable (or functional). Nonviability (nonfunctionality) breaks the chain. Second, evolutionary time is not sufficient to search sequence space exhaustively. However, synthetic protein design does not evolve in the Darwinian sense and therefore doesn't have to descend from another successful design, and this greatly expands the possibilities for protein design.

RosettaDesign from the Baker group at the University of Washington is a model case for how protein design software works. One begins with some understanding of how the backbone conformation of a protein relates to whatever function is being designed or engineered (for example how it forms or doesn't form a properly folded structure, binding pocket, catalytic site, etc.). The program takes the spatial position of a desired protein backbone as input. It then searches all possible amino acid sequences to find those that have the minimum energy for the given backbone conformation. The energy model is a combination of semiempirical (Lennard-Jones) and fully empirical (implicit solvation) models. The current version of RosettaDesign not only can search all possible sequences, but determines whether or not each sequence will be stable in the target conformation, discarding those sequences that are not (Kuhlman et al. 2003, Science 302: 1364-1368).

Generally, the invention provides polynucleotide, protein, and library production techniques that may be used in various fields and contexts to produce useful biological constructs. Exemplary uses for protein design include, for example, design of proteins having novel characteristics including biochemical and/or biophysical properties. Another example is for the design of novel catalytic RNAs. In one embodiment, the methods described herein may be used to develop improved human therapeutics, for example, by designing backbones around active site residues and mutating residues in silico to produce variants with desired characteristics such as higher binding affinity, improved stability, lower immunogenicity, better bioavailability, or ease of manufacture while maintaining functionality. In another embodiment, the methods described herein may be used to develop novel industrial enzymes, for example, by designing active sites to carry out desired chemical transformation, and then designing a backbone scaffold to hold the novel active site in an active conformation. Exemplary applications for industrial enzymes include chemical synthesis, pulp and paper bleaching, conversion of biomass to energy, etc. In another embodiment, the methods disclosed herein may be used to develop bi-functional or multifunctional proteins. For example, multivalent, high-affinity binders, may be developed by designing linkers to optimally connect binding domains yielding a construct with, e.g., the highest possible affinity, or a slow off rate. Additionally, the methods described herein may be used to develop combinations of a binding domain, linker and catalytic domain that result in optimal catalytic efficiency. In yet another embodiment, the methods described herein may be used to develop “minimal proteins.” For example, the backbone of the functional area(s) of a protein may be fixed and the chains of this region may be connected with the smallest possible backbone that results in a single, stable molecule. The sequence of the polypeptide may be further optimized to maintain the structure of the backbone. Such minimal proteins may facilitate protein manufacturing and yield proteins with greater stability or higher rates of diffusion.

In an exemplary embodiment, large numbers of protein design variants may be expressed and subjected to a screen, or preferably a selection process, to identify variants exhibiting a desired characteristic. In various embodiments, at least about 10, 100, 1,000, 10,000, 100,000 or more variants may be screened for a desired characteristic. Such variants may optionally be selected based on an in silico prescreen that produces a rank ordered list of variants obtained from analysis of a large library of possible variants.

By computationally screening very large libraries of mutants (variants), greater diversity of protein sequences can be screened (i.e. a larger sampling of sequence space), leading to greater improvements in protein function. Further, fewer mutants may need to be tested experimentally to screen a given library size, reducing the cost and difficulty of protein engineering. By using computational methods to pre-screen a protein library, the computational features of speed and efficiency are combined with the ability of experimental library screening to create new activities in proteins for which appropriate computational models and structure-function relationships are unclear.

In addition, as is more fully outlined below, the libraries may be biased in any number of ways, allowing the generation of libraries that vary in their focus; for example, domains, individual residues, surface residues, subsets of residues, active or binding sites, etc., may all be varied or kept constant as desired.

Accordingly, the present invention provides methods for generating secondary libraries of scaffold protein variants. Protein as used herein is meant to encompass at least two amino acids linked together by a peptide bond, including, polypeptides, oligopeptides, peptides and variously derivatized polypeptides such as phosphorylated or glycosylated proteins. The peptidyl group may comprise naturally occurring amino acids and peptide bonds, or synthetic peptidomimetic structures, i.e. “analogs”, such as peptoids (see Simon et al., PNAS USA 89(20):9367 (1992)). The amino acids may either be naturally occurring or non-naturally occurring; as will be appreciated by those in the art, any structure for which a set of rotamers is known or can be generated can be used as an amino acid. The side chains may be in either the (R) or the (S) configuration. In a preferred embodiment, the amino acids are in the (S) or L-configuration.

The scaffold protein may be any protein, but preferred proteins are those for which a three dimensional structure is known or can be generated; that is, for which there are three dimensional coordinates for each atom of the protein. Generally this can be determined using X-ray crystallographic techniques, NMR techniques, de novo modeling, homology modeling, etc. In general, if X-ray structures are used, structures at 2 Å resolution or better are preferred, but not required.

Computational or in silico methods are available to assist with predicting secondary structure. Where a crystal structure is unavailable, computer modeling can be generated through a series of alignments and extrapolations from known structures and sequences of related proteins and their interactions with each other—i.e., homology modeling. For example, two polypeptides or proteins that have a sequence identity of greater than 30%, or similarity greater than 40% often have similar structural topologies. The protein structural database (PDB) has provides increasing predictability of secondary structure, including the potential number of folds within a polypeptide's or protein's structure. See Holm et al., 1999, Nucl. Acid. Res. 27:244-247. It has been suggested (Brenner et al., 1997, Curr. Op. Struct. Biol. 7:369-376) that there are a limited number of folds in a given polypeptide or protein and that once a critical number of structures have been resolved, structural prediction will become dramatically more accurate.

The scaffold proteins may be from any organism, including prokaryotes and eukaryotes, with enzymes from bacteria, fungi, extremeophiles such as the archebacteria, insects, fish, animals (particularly mammals and particularly human) and birds all possible.

Thus, by “scaffold protein” herein is meant a protein for which a library of variants is desired. As will be appreciated by those in the art, any number of scaffold proteins find use in the present invention. Specifically included within the definition of “protein” are fragments and domains of known proteins, including functional domains such as enzymatic domains, binding domains, etc., and smaller fragments, such as turns, loops, etc. That is, portions of proteins may be used as well. In addition, “protein” as used herein includes proteins, oligopeptides and peptides. In addition, protein variants, i.e. non-naturally occurring protein analog structures, may be used. Suitable proteins include, but are not limited to, industrial and pharmaceutical proteins, including ligands, cell surface receptors, antigens, antibodies, cytokines, hormones, transcription factors, signaling modules, cytoskeletal proteins and enzymes. Suitable classes of enzymes include, but are not limited to, hydrolases such as proteases, carbohydrases, lipases; isomerases such as racemases, epimerases, tautomerases, or mutases; transferases, kinases, oxidoreductases, and phophatases. Suitable enzymes are listed in the Swiss-Prot enzyme database. Suitable protein backbones include, but are not limited to, all of those found in the protein data base compiled and serviced by the Research Collaboratory for Structural Bioinformatics (RCSB, formerly the Brookhaven National Lab).

Exemplary scaffold proteins include, but are not limited to, those with known structures (including variants) including cytokines (IL-1ra (+receptor complex), IL-1 (receptor alone), IL-1a, IL-1b (including variants and or receptor complex), IL-2, IL-3, IL-4, IL-5, IL-6, IL-8, IL-10, IFN-β, INF-γ, IFN-α-2a; IFN-α-2B, TNF-α.; CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration Inhibitory Factor, Human Glycosylation-Inhibiting Factor, Human Rantes, Human Macrophage Inflammatory Protein 1 Beta, human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor A, Vascular Endothelial growth factor (VEGF), acidic Fibroblast growth factor, basic Fibroblast growth factor, Endothelial growth factor, Nerve growth factor, Brain Derived Neurotrophic Factor, Ciliary Neurotrophic Factor, Platelet Derived Growth Factor, Human Hepatocyte Growth Factor, Glial Cell-Derived Neurotrophic Factor, (as well as the at least 55 cytokines in PDB)); Erythropoietin; other extracellular signaling moieties, including, but not limited to, hedgehog Sonic, hedgehog Desert, hedgehog Indian, hCG; coaguation factors including, but not limited to, TPA and Factor VIIa; transcription factors, including but not limited to, p53, p53 tetramerization domain, Zn fingers (of which more than 12 have structures), homeodomains (of which 8 have structures), leucine zippers (of which 4 have structures); antibodies, including, but not limited to, cFv; viral proteins, including, but not limited to, hemagglutinin trimerization domain and hiv Gp41 ectodomain (fusion domain); intracellular signaling modules, including, but not limited to, SH2 domains (of which 8 structures are known), SH3 domains (of which 11 have structures), and Pleckstin Homology Domains; receptors, including, but not limited to, the extracellular Region Of Human Tissue Factor Cytokine-Binding Region Of Gp130, G-CSF receptor, erythropoietin receptor, Fibroblast Growth Factor receptor, TNF receptor, IL-1 receptor, IL-1 receptor/IL1ra complex, IL-4 receptor, INF-γ receptor alpha chain, MHC Class I, MHC Class II, T Cell Receptor, Insulin receptor, insulin receptor tyrosine kinase and human growth hormone receptor.

Once a scaffold protein is chosen, a library may be generated, typically using known or to be developed computational processing techniques. Generally speaking, in some embodiments, the goal of the computational processing is to determine a set of optimized protein sequences. By “optimized protein sequence” herein is meant a sequence that best fits the mathematical equations of the computational process. As will be appreciated by those in the art, a global optimized sequence is the one sequence that best fits the equations (for example, when protein design automation (PDA) is used, the global optimized sequence is the sequence that best fits Equation 1, below); i.e. the sequence that has the lowest energy of any possible sequence. However, there are any number of sequences that are not the global minimum but that have low energies.

The libraries can be generated in a variety of ways. In essence, any methods that can result in either the relative ranking of the possible sequences of a protein based on measurable stability parameters, or a list of suitable sequences can be used. As will be appreciated by those in the art, any of the methods described herein or known in the art may be used alone, or in combination with other methods.

Generally, there are a variety of computational methods that can be used to generate a library. In a preferred embodiment, sequence based methods are used. Alternatively, structure based methods, such as protein design automation (PDA), described in detail below, are used.

In a preferred embodiment, the scaffold protein is an enzyme and highly accurate electrostatic models can be used for enzyme active site residue scoring to improve enzyme active site libraries (see Warshel, Computer Modeling of Chemical Reactions in Enzymes and Solutions, Wiley & Sons, New York, (1991), hereby expressly incorporated by reference). These accurate models can assess the relative energies of sequences with high precision, but are computationally intensive.

Similarly, molecular dynamics calculations can be used to computationally screen sequences by individually calculating mutant sequence scores and compiling a rank ordered list.

In a preferred embodiment, residue pair potentials can be used to score sequences (Miyazawa et al., Macromolecules 18(3):534-552 (1985), expressly incorporated by reference) during computational screening.

In a preferred embodiment, sequence profile scores (Bowie et al., Science 253(5016):164-70 (1991), incorporated by reference) and/or potentials of mean force (Hendlich et al., J. Mol. Biol. 216(1):167-180 (1990), also incorporated by reference) can also be calculated to score sequences. These methods assess the match between a sequence and a 3D protein structure and hence can act to screen for fidelity to the protein structure. By using different scoring functions to rank sequences, different regions of sequence space can be sampled in the computational screen.

Furthermore, scoring functions can be used to screen for sequences that would create metal or co-factor binding sites in the protein (Helling a, Fold Des. 3(1):R1-8 (1998), hereby expressly incorporated by reference). Similarly, scoring functions can be used to screen for sequences that would create disulfide bonds in the protein. These potentials attempt to specifically modify a protein structure to introduce a new structural motif.

In a preferred embodiment, sequence and/or structural alignment programs can be used to generate libraries. As is known in the art, there are a number of sequence-based alignment programs; including for example, Smith-Waterman searches, Needleman-Wunsch, Double Affine Smith-Waterman, frame search, Gribskov/GCG profile search, Gribskov/GCG profile scan, profile frame search, Bucher generalized profiles, Hidden Markov models, Hframe, Double Frame, Blast, Psi-Blast, Clustal, and GeneWise.

The source of the sequences can vary widely, and include taking sequences from one or more of the known databases, including, but not limited to, SCOP (Hubbard, et al., Nucleic Acids Res 27(1):254-256. (1999)); PFAM (Bateman, et al., Nucleic Acids Res 27(1):260-262. (1999)); VAST (Gibrat, et al., Curr Opin Struct Biol 6(3):377-385. (1996)); CATH (Orengo, et al., Structure 5(8):1093-1108. (1997)); PhD Predictor (world wide web at embl-heidelberg.de/predictprotein/predictprotein.html); Prosite (Hofmann, et al., Nucleic Acids Res 27(1):215-219. (1999)); PIR (world wide web at mips.biochem.mpg.de/proj/protseqdb/); GenBank (world wide web at ncbi.nlm.nih.gov/); PDB (world wide web at rcsb.org) and BIND (Bader, et al., Nucleic Acids Res 29(1):242-245 (2001)).

In addition, sequences from these databases can be subjected to contiguous analysis or gene prediction; see Wheeler, et al., Nucleic Acids Res 28(1):10-14. (2000) and Burge and Karlin, J Mol Biol 268(1):78-94. (1997).

As is known in the art, there are a number of sequence alignment methodologies that can be used. For example, sequence homology based alignment methods can be used to create sequence alignments of proteins related to the target structure (Altschul et al., J. Mol. Biol. 215(3):403 (1990), incorporated by reference). These sequence alignments are then examined to determine the observed sequence variations. These sequence variations are tabulated to define a primary library. In addition, as is further outlined below, these methods can also be used to generate secondary libraries.

Sequence based alignments can be used in a variety of ways. For example, a number of related proteins can be aligned, as is known in the art, and the “variable” and “conserved” residues defined; that is, the residues that vary or remain identical between the family members can be defined. These results can be used to generate a probability table. Alternatively, the allowed sequence variations can be used to define the amino acids considered at each position during the computational screening. Another variation is to bias the score for amino acids that occur in the sequence alignment, thereby increasing the likelihood that they are found during computational screening but still allowing consideration of other amino acids. This bias would result in a focused primary library but would not eliminate from consideration amino acids not found in the alignment. In addition, a number of other types of bias may be introduced. For example, diversity may be forced; that is, a “conserved” residue is chosen and altered to force diversity on the protein and thus sample a greater portion of the sequence space. Alternatively, the positions of high variability between family members (i.e. low conservation) can be randomized, either using all or a subset of amino acids. Similarly, outlier residues, either positional outliers or side chain outliers, may be eliminated.

Similarly, structural alignment of structurally related proteins can be done to generate sequence alignments. There are a wide variety of such structural alignment programs known. See for example VAST from the NCBI (world wide web at ncbi.nlm.nih.gov:80/StructureNAST/vast.shtml); SSAP (Orengo and Taylor, Methods Enzymol 266(617-635 (1996)) SARF2 (Alexandrov, Protein Eng 9(9):727-732. (1996)) CE (Shindyalov and Bourne, Protein Eng 11(9):739-747. (1998)); (Orengo et al., Structure 5(8):1093-108 (1997); Dali (Holm et al., Nucleic Acid Res. 26(1):316-9 (1998), all of which are incorporated by reference). These structurally-generated sequence alignments can then be examined to determine the observed sequence variations.

In certain embodiments, libraries can be generated by predicting secondary structure from sequence, and then selecting sequences that are compatible with the predicted secondary structure. There are a number of secondary structure prediction methods, including, but not limited to, threading (Bryant and Altschul, Curr Opin Struct Biol 5(2):236-244. (1995)), Profile 3D (Bowie, et al., Methods Enzymol 266(598-616 (1996); MONSSTER (Skolnick, et al., J Mol Biol 265(2):217-241. (1997); Rosetta (Simons, et al., Proteins 37(53):171-176 (1999); PSI-BLAST (Altschul and Koonin, Trends Biochem Sci 23(11):444-447. (1998)); Impala (Schaffer, et al., Bioinformatics 15(12):1000-1011. (1999)); HMMER (McClure, et al., Proc Int Conf Intell Syst Mol Biol 4(155-164 (1996)); Clustal W (world wide web at ebi.ac.uk/clustalw/); BLAST (Altschul, et al., J Mol Biol 215(3):403-410. (1990)), helix-coil transition theory (Munoz and Serrano, Biopolymers 41:495, 1997), neural networks, local structure alignment and others (e.g., see in Selbig et al., Bioinformatics 15:1039, 1999).

Similarly, as outlined above, other computational methods are known, including, but not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Helling a and Richards, PNAS USA 91: 5803-5807 (1994)); and residue pair potentials (Jones, Protein Science 3: 567-574, (1994); PROSA (Heindlich et al., J. Mol. Biol. 216:167-180 (1990); THREADER (Jones et al., Nature 358:86-89 (1992), and other inverse folding methods such as those described by Simons et al. (Proteins, 34:535-543, 1999), Levitt and Gerstein (PNAS USA, 95:5913-5920, 1998), Godzik et al., PNAS, V89, PP 12098-102; Godzik and Skolnick (PNAS USA, 89:12098-102, 1992), Godzik et al. (J. Mol. Biol. 227:227-38, 1992) and two profile methods (Gribskov et al. PNAS 84:4355-4358 (1987) and Fischer and Eisenberg, Protein Sci. 5:947-955 (1996), Rice and Eisenberg J. Mol. Biol. 267:1026-1038 (1997)), all of which are expressly incorporated by reference. In addition, other computational methods such as those described by Koehl and Levitt (J. Mol. Biol. 293:1161-1181 (1999); J. Mol. Biol. 293:1183-1193 (1999); expressly incorporated by reference) can be used to create a protein sequence library for improved properties and function.

In addition, there are computational methods based on forcefield calculations such as SCMF that can be used as well for SCMF, see Delarue et la. Pac. Symp. Biocomput. 109-21 (1997), Koehl et al., J. Mol. Biol. 239:249 (1994); Koehl et al., Nat. Struc. Biol. 2:163 (1995); Koehl et al., Curr. Opin. Struct. Biol. 6:222 (1996); Koehl et al., J. Mol. Bio. 293:1183 (1999); Koehl et al., J. Mol. Biol. 293:1161 (1999); Lee J. Mol. Biol. 236:918 (1994); and Vasquez Biopolymers 36:53-70 (1995); all of which are expressly incorporated by reference. Other forcefield calculations that can be used to optimize the conformation of a sequence within a computational method, or to generate de novo optimized sequences as outlined herein include, but are not limited to, OPLS-AA (Jorgensen, et al., J. Am. Chem. Soc. (1996), v 118, pp 11225-11236; Jorgensen, W. L.; BOSS, Version 4.1; Yale University: New Haven, Conn. (1999)); OPLS (Jorgensen, et al., J. Am. Chem. Soc. (1988), v 110, pp 1657ff; Jorgensen, et al., J. Am. Chem. Soc. (1990), v 112, pp 4768ff); UNRES (United Residue Forcefield; Liwo, et al., Protein Science (1993), v 2, pp 1697-1714; Liwo, et al., Protein Science (1993), v 2, pp 1715-1731; Liwo, et al., J. Comp. Chem. (1997), v 18, pp 849-873; Liwo, et al., J. Comp. Chem. (1997), v 18, pp 874-884; Liwo, et al., J. Comp. Chem. (1998), v 19, pp 259-276; Forcefield for Protein Structure Prediction (Liwo, et al., Proc. Natl. Acad. Sci. USA (1999), v 96, pp 5482-5485); ECEPP/3 (Liwo et al., J Protein Chem 1994 May; 13(4): 375-80); AMBER 1.1 force field (Weiner, et al., J. Am. Chem. Soc. v 106, pp 765-784); AMBER 3.0 force field (U. C. Singh et al., Proc. Natl. Acad. Sci. USA. 82:755-759); CHARMM and CHARMM22 (Brooks, et al., J. Comp. Chem. v 4, pp 187-217); cvff.0 (Dauber-Osguthorpe, et al., (1988) Proteins: Structure, Function and Genetics, v 4, pp 31-47); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference.

In a preferred embodiment, the computational method used to generate the primary library is Protein Design Automation (PDA), as is described in U.S. Pat. No. 6,269,312 and PCT Publication No. WO 98/47089, both of which are expressly incorporated herein by reference. Briefly, PDA can be described as follows. A known protein structure is used as the starting point. The residues to be optimized are then identified, which may be the entire sequence or subset(s) thereof. The side chains of any positions to be varied are then removed. The resulting structure consisting of the protein backbone and the remaining sidechains is called the template. Each variable residue position is then preferably classified as a core residue, a surface residue, or a boundary residue; each classification defines a subset of possible amino acid residues for the position (for example, core residues generally will be selected from the set of hydrophobic residues, surface residues generally will be selected from the hydrophilic residues, and boundary residues may be either). Each amino acid can be represented by a discrete set of all allowed conformers of each side chain, called rotamers. Thus, to arrive at an optimal sequence for a backbone, all possible sequences of rotamers must be screened, where each backbone position can be occupied either by each amino acid in all its possible rotameric states, or a subset of amino acids, and thus a subset of rotamers.

Two sets of interactions are then calculated for each rotamer at every position: the interaction of the rotamer side chain with all or part of the backbone (the “singles” energy, also called the rotamer/template or rotamer/backbone energy), and the interaction of the rotamer side chain with all other possible rotamers at every other position or a subset of the other positions (the “doubles” energy, also called the rotamer/rotamer energy). The energy of each of these interactions is calculated through the use of a variety of scoring functions, which include the energy of van der Waal's forces, the energy of hydrogen bonding, the energy of secondary structure propensity, the energy of surface area solvation and the electrostatics. Thus, the total energy of each rotamer interaction, both with the backbone and other rotamers, is calculated, and stored in a matrix form.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, a “Dead End Elimination” (DEE) calculation is performed. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably. DEE can be rerun comparing pairs of rotamers, or combinations of rotamers, which will eventually result in the determination of a single sequence which represents the global optimum energy.

Once the global solution has been found, a Monte Carlo search may be done to generate a rank-ordered list of sequences in the neighborhood of the DEE solution. Starting at the DEE solution, random positions are changed to other rotamers, and the new sequence energy is calculated. If the new sequence meets the criteria for acceptance, it is used as a starting point for another jump. After a predetermined number of jumps, a rank-ordered list of sequences is generated. Monte Carlo searching is a sampling technique to explore sequence space around the global minimum or to find new local minima distant in sequence space. As is more additionally outlined below, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Similarly, for all the sampling techniques, the acceptance criteria of whether a sampling jump is accepted can be altered.

As outlined in U.S. Pat. No. 6,269,312, the protein backbone (comprising (for a naturally occurring protein) the nitrogen, the carbonyl carbon, the α-carbon, and the carbonyl oxygen, along with the direction of the vector from the α-carbon to the β-carbon) may be altered prior to the computational analysis, by varying a set of parameters called supersecondary structure parameters.

Once a protein structure backbone is generated (with alterations, as outlined above) and input into the computer, explicit hydrogens are added if not included within the structure (for example, if the structure was generated by X-ray crystallography, hydrogens must be added). After hydrogen addition, energy minimization of the structure is run, to relax the hydrogens as well as the other atoms, bond angles and bond lengths. In a preferred embodiment, this is done by doing a number of steps of conjugate gradient minimization (Mayo et al., J. Phys. Chem. 94:8897 (1990)) of atomic coordinate positions to minimize the Dreiding force field with no electrostatics. Generally from about 10 to about 250 steps is preferred, with about 50 being most preferred.

The protein backbone structure contains at least one variable residue position. As is known in the art, the residues, or amino acids, of proteins are generally sequentially numbered starting with the N-terminus of the protein. Thus a protein having a methionine at it's N-terminus is said to have a methionine at residue or amino acid position 1, with the next residues as 2, 3, 4, etc. At each position, the wild type (i.e. naturally occurring) protein may have one of at least about 20 amino acids, in any number of rotamers. By “variable residue position” herein is meant an amino acid position of the protein to be designed that is not fixed in the design method as a specific residue or rotamer, generally the wild-type residue or rotamer.

In a preferred embodiment, all of the residue positions of the protein are variable. That is, every amino acid side chain may be altered in the methods of the present invention. This is particularly desirable for smaller proteins, although the present methods allow the design of larger proteins as well. While there is no theoretical limit to the length of the protein which may be designed this way, there is a practical computational limit.

In an alternate preferred embodiment, only some of the residue positions of the protein are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable (for example, to eliminate a proteolytic site or alter the substrate specificity of an enzyme), the residue may be fixed as a particular amino acid. Alternatively, the methods of the present invention may be used to evaluate mutations de novo, as is discussed below. In an alternate preferred embodiment, a fixed position may be “floated”; the amino acid at that position is fixed, but different rotamers of that amino acid are tested. In this embodiment, the variable residues may be at least one, or anywhere from 0.1% to 99.9% of the total number of residues. Thus, for example, it may be possible to change only a few (or one) residues, or most of the residues, with all possibilities in between.

In a preferred embodiment, residues which can be fixed include, but are not limited to, structurally or biologically functional residues; alternatively, biologically functional residues may specifically not be fixed. For example, residues which are known to be important for biological activity, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, metal binding sites, critical hydrogen bonding residues, residues critical for backbone conformation such as proline or glycine, residues critical for packing interactions, etc. may all be fixed in a conformation or as a single rotamer, or “floated”.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

In one embodiment, each variable position is classified as either a core, surface or boundary residue position, although in some cases, as explained below, the variable position may be set to glycine to minimize backbone strain. In addition, as outlined herein, residues need not be classified, they can be chosen as variable and any set of amino acids may be used. Any combination of core, surface and boundary positions can be utilized: core, surface and boundary residues; core and surface residues; core and boundary residues, and surface and boundary residues, as well as core residues alone, surface residues alone, or boundary residues alone.

The classification of residue positions as core, surface or boundary may be done in several ways, as will be appreciated by those in the art. In a preferred embodiment, the classification is done via a visual scan of the original protein backbone structure, including the side chains, and assigning a classification based on a subjective evaluation of one skilled in the art of protein modeling. Alternatively, a preferred embodiment utilizes an assessment of the orientation of the Cα-Cβ vectors relative to a solvent accessible surface computed using only the template Cα atoms, as outlined in U.S. Pat. No. 6,269,312 and PCT Publication No. WO 98/47089. Alternatively, a surface area calculation can be done.

Once each variable position is classified as either core, surface or boundary, a set of amino acid side chains, and thus a set of rotamers, is assigned to each position. That is, the set of possible amino acid side chains that the program will allow to be considered at any particular position is chosen. Subsequently, once the possible amino acid side chains are chosen, the set of rotamers that will be evaluated at a particular position can be determined. Thus, a core residue will generally be selected from the group of hydrophobic residues consisting of alanine, valine, isoleucine, leucine, phenylalanine, tyrosine, tryptophan, and methionine (in some embodiments, when the a scaling factor of the van der Waals scoring function, described below, is low, methionine is removed from the set), and the rotamer set for each core position potentially includes rotamers for these eight amino acid side chains (all the rotamers if a backbone independent library is used, and subsets if a rotamer dependent backbone is used). Similarly, surface positions are generally selected from the group of hydrophilic residues consisting of alanine, serine, threonine, aspartic acid, asparagine, glutamine, glutamic acid, arginine, lysine and histidine. The rotamer set for each surface position thus includes rotamers for these ten residues. Finally, boundary positions are generally chosen from alanine, serine, threonine, aspartic acid, asparagine, glutamine, glutamic acid, arginine, lysine histidine, valine, isoleucine, leucine, phenylalanine, tyrosine, tryptophan, and methionine. The rotamer set for each boundary position thus potentially includes every rotamer for these seventeen residues (assuming cysteine, glycine and proline are not used, although they can be). Additionally, in some preferred embodiments, a set of 18 naturally occurring amino acids (all except cysteine and proline, which are known to be particularly disruptive) are used.

Thus, as will be appreciated by those in the art, there is a computational benefit to classifying the residue positions, as it decreases the number of calculations. It should also be noted that there may be situations where the sets of core, boundary and surface residues are altered from those described above; for example, under some circumstances, one or more amino acids is either added or subtracted from the set of allowed amino acids. For example, some proteins which dimerize or multimerize, or have ligand binding sites, may contain hydrophobic surface residues, etc. In addition, residues that do not allow helix “capping” or the favorable interaction with an α-helix dipole may be subtracted from a set of allowed residues. This modification of amino acid groups is done on a residue by residue basis.

In a preferred embodiment, proline, cysteine and glycine are not included in the list of possible amino acid side chains, and thus the rotamers for these side chains are not used. However, in a preferred embodiment, when the variable residue position has a particular angle (that is, the dihedral angle defined by 1) the carbonyl carbon of the preceding amino acid; 2) the nitrogen atom of the current residue; 3) the α-carbon of the current residue; and 4) the carbonyl carbon of the current residue) greater than 0°, the position is set to glycine to minimize backbone strain.

Once the group of potential rotamers is assigned for each variable residue position, processing proceeds as outlined in U.S. Pat. No. 6,269,312 and PCT Publication No. WO 98/47089. This processing step entails analyzing interactions of the rotamers with each other and with the protein backbone to generate optimized protein sequences. Simplistically, the processing initially comprises the use of a number of scoring functions to calculate energies of interactions of the rotamers, either to the backbone itself or other rotamers. Preferred PDA scoring functions include, but are not limited to, a Van der Waals potential scoring function, a hydrogen bond potential scoring function, an atomic solvation scoring function, a secondary structure propensity scoring function and an electrostatic scoring function. As is further described below, at least one scoring function is used to score each position, although the scoring functions may differ depending on the position classification or other considerations, like favorable interaction with an α-helix dipole. As outlined below, the total energy which is used in the calculations is the sum of the energy of each scoring function used at a particular position, as is generally shown in Equation 1: Eₜₒₜₐₗ=nE_{vdw}+nEₐₛ+nEₕ-bonding+nEₛₛ+nEₑₗₑ−c Equation 1.

In Equation 1, the total energy is the sum of the energy of the van der Waals potential (E_{vdw}), the energy of atomic solvation (Eₐₛ), the energy of hydrogen bonding (E_{h-bonding}), the energy of secondary structure (Eₛₛ) and the energy of electrostatic interaction (E_{elec}). The term n is either 0 or 1, depending on whether the term is to be considered for the particular residue position.

As outlined in U.S. Pat. No. 6,269,312 and PCT Publication No. WO 98/47089, any combination of these scoring functions, either alone or in combination, may be used. Once the scoring functions to be used are identified for each variable position, the preferred first step in the computational analysis comprises the determination of the interaction of each possible rotamer with all or part of the remainder of the protein. That is, the energy of interaction, as measured by one or more of the scoring functions, of each possible rotamer at each variable residue position with either the backbone or other rotamers, is calculated. In a preferred embodiment, the interaction of each rotamer with the entire remainder of the protein, i.e. both the entire template and all other rotamers, is done. However, as outlined above, it is possible to only model a portion of a protein, for example a domain of a larger protein, and thus in some cases, not all of the protein need be considered. The term “portion”, as used herein, with regard to a protein refers to a fragment of that protein. This fragment may range in size from 10 amino acid residues to the entire amino acid sequence minus one amino acid. Accordingly, the term “portion”, as used herein, with regard to a nucleic refers to a fragment of that nucleic acid. This fragment may range in size from 10 nucleotides to the entire nucleic acid sequence minus one nucleotide.

In a preferred embodiment, the first step of the computational processing is done by calculating two sets of interactions for each rotamer at every position: the interaction of the rotamer side chain with the template or backbone (the “singles” energy), and the interaction of the rotamer side chain with all other possible rotamers at every other position (the “doubles” energy), whether that position is varied or floated. It should be understood that the backbone in this case includes both the atoms of the protein structure backbone, as well as the atoms of any fixed residues, wherein the fixed residues are defined as a particular conformation of an amino acid.

Thus, “singles” (rotamer/template) energies are calculated for the interaction of every possible rotamer at every variable residue position with the backbone, using some or all of the scoring functions. Thus, for the hydrogen bonding scoring function, every hydrogen bonding atom of the rotamer and every hydrogen bonding atom of the backbone is evaluated, and the EHB is calculated for each possible rotamer at every variable position. Similarly, for the van der Waals scoring function, every atom of the rotamer is compared to every atom of the template (generally excluding the backbone atoms of its own residue), and the E_{vdw} is calculated for each possible rotamer at every variable residue position. In addition, generally no van der Waals energy is calculated if the atoms are connected by three bonds or less. For the atomic salvation scoring function, the surface of the rotamer is measured against the surface of the template, and the Eₐₛ for each possible rotamer at every variable residue position is calculated. The secondary structure propensity scoring function is also considered as a singles energy, and thus the total singles energy may contain an Eₛₛ term. As will be appreciated by those in the art, many of these energy terms will be close to zero, depending on the physical distance between the rotamer and the template position; that is, the farther apart the two moieties, the lower the energy.

For the calculation of “doubles” energy (rotamer/rotamer), the interaction energy of each possible rotamer is compared with every possible rotamer at all other variable residue positions. Thus, “doubles” energies are calculated for the interaction of every possible rotamer at every variable residue position with every possible rotamer at every other variable residue position, using some or all of the scoring functions. Thus, for the hydrogen bonding scoring function, every hydrogen bonding atom of the first rotamer and every hydrogen bonding atom of every possible second rotamer is evaluated, and the E_{HB} is calculated for each possible rotamer pair for any two variable positions. Similarly, for the van der Waals scoring function, every atom of the first rotamer is compared to every atom of every possible second rotamer, and the E_{vdw} is calculated for each possible rotamer pair at every two variable residue positions. For the atomic solvation scoring function, the surface of the first rotamer is measured against the surface of every possible second rotamer, and the Eₐₛ for each possible rotamer pair at every two variable residue positions is calculated. The secondary structure propensity scoring function need not be run as a “doubles” energy, as it is considered as a component of the “singles” energy. As will be appreciated by those in the art, many of these double energy terms will be close to zero, depending on the physical distance between the first rotamer and the second rotamer; that is, the farther apart the two moieties, the lower the energy.

In addition, as will be appreciated by those in the art, a variety of force fields that can be used in the PCA calculations can be used, including, but not limited to, Dreiding I and Dreiding II (Mayo et al, J. Phys. Chem. 948897 (1990)), AMBER (Weiner et al., J. Amer. Chem. Soc. 106:765 (1984) and Weiner et al., J. Comp. Chem. 106:230 (1986)), MM2 (Allinger J. Chem. Soc. 99:8127 (1977), Liljefors et al., J. Corn. Chem. 8:1051 (1987)); MMP2 (Sprague et al., J. Comp. Chem. 8:581 (1987)); CHARMM (Brooks et al., J. Comp. Chem. 106:187 (1983)); GROMOS; and MM3 (Allinger et al., J. Amer. Chem. Soc. 111:8551 (1989)), OPLS-M (Jorgensen, et al., J. Am. Chem. Soc. (1996), v 118, pp 11225-11236; Jorgensen, W. L.; BOSS, Version 4.1; Yale University: New Haven, Conn. (1999)); OPLS (Jorgensen, et al., J. Am. Chem. Soc. (1988), v 110, pp 1657ff; Jorgensen, et al., J. Am. Chem. Soc. (1990), v 112, pp 4768ff); UNRES (United Residue Forcefield; Liwo, et al., Protein Science (1993), v 2, pp 1697-1714; Liwo, et al., Protein Science (1993), v 2, pp 1715-1731; Liwo, et al., J. Comp. Chem. (1997), v 18, pp 849-873; Liwo, et al., J. Comp. Chem. (1997), v 18, pp 874-884; Liwo, et al., J. Comp. Chem. (1998), v 19, pp 259-276; Forcefield for Protein Structure Prediction (Liwo, et al., Proc. Natl. Acad. Sci. USA (1999), v 96, pp 482-5485); ECEPP/3 (Liwo et al., J Protein Chem 1994 May; 13(4):375-80); AMBER 1.1 force field (Weiner, et al., J. Am. Chem. Soc. v106, pp 765-784); AMBER 3.0 force field (U. C. Singh et al., Proc. Natl. Acad. Sci. USA. 82:755-759); CHARMM and CHARMM22 (Brooks, et al., J. Comp. Chem. v 4, pp 187-217); cvff3.0 (Dauber-Osguthorpe, et al., (1988) Proteins: Structure, Function and Genetics, v 4, pp 31-47); cff91 (Maple, et al., J. Comp. Chem. v 15, pp 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference.

Once the singles and doubles energies are calculated and stored, the next step of the computational processing may occur. As outlined in U.S. Pat. No. 6,269,312 and PCT Publication No. WO 98/47089, preferred embodiments utilize a Dead End Elimination (DEE) step, and preferably a Monte Carlo step.

PDA, viewed broadly, has three components that may be varied to alter the output (e.g. the library): the scoring functions used in the process; the filtering technique, and the sampling technique.

In a preferred embodiment, the scoring functions may be altered. In a preferred embodiment, the scoring functions outlined above may be biased or weighted in a variety of ways. For example, a bias towards or away from a reference sequence or family of sequences can be done; for example, a bias towards wild-type or homolog residues may be used. Similarly, the entire protein or a fragment of it may be biased; for example, the active site may be biased towards wild-type residues, or domain residues towards a particular desired physical property can be done. Furthermore, a bias towards or against increased energy can be generated. Additional scoring function biases include, but are not limited to applying electrostatic potential gradients or hydrophobicity gradients, adding a substrate or binding partner to the calculation, or biasing towards a desired charge or hydrophobicity.

In addition, in an alternative embodiment, there are a variety of additional scoring functions that may be used. Additional scoring functions include, but are not limited to torsional potentials, or residue pair potentials, or residue entropy potentials. Such additional scoring functions can be used alone, or as functions for processing the library after it is scored initially. For example, a variety of functions derived from data on binding of peptides to MHC (Major Histocompatibility Complex) can be used to rescore a library in order to eliminate proteins containing sequences which can potentially bind to MHC, i.e. potentially immunogenic sequences.

In a preferred embodiment, a variety of filtering techniques can be done, including, but not limited to, DEE and its related counterparts. Additional filtering techniques include, but are not limited to branch-and-bound techniques for finding optimal sequences (Gordon and Majo, Structure Fold. Des. 7:1089-98, 1999), and exhaustive enumeration of sequences. It should be noted however, that some techniques may also be done without any filtering techniques; for example, sampling techniques can be used to find good sequences, in the absence of filtering.

As will be appreciated by those in the art, once an optimized sequence or set of sequences is generated, (or again, these need not be optimized or ordered) a variety of sequence space sampling methods can be done, either in addition to the preferred Monte Carlo methods, or instead of a Monte Carlo search. That is, once a sequence or set of sequences is generated, preferred methods utilize sampling techniques to allow the generation of additional, related sequences for testing.

These sampling methods can include the use of amino acid substitutions, insertions or deletions, or recombinations of one or more sequences. As outlined herein, a preferred embodiment utilizes a Monte Carlo search, which is a series of biased, systematic, or random jumps. However, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Jumps where multiple residue positions are coupled (two residues always change together, or never change together), jumps where whole sets of residues change to other sequences (e.g., recombination). Similarly, for all the sampling techniques, the acceptance criteria of whether a sampling jump is accepted can be altered, to allow broad searches at high temperature and narrow searches close to local optima at low temperatures. See Metropolis et al., J. Chem Phys v 21, pp 1087, 1953, hereby expressly incorporated by reference.

In a preferred embodiment, particularly for longer proteins or proteins for which large samples are desired, the library sequences are used to create nucleic acids such as DNA which encode the member sequences and which can then be cloned into host cells, expressed and assayed, if desired. Thus, nucleic acids, and particularly DNA, can be made which encodes each member protein sequence using the methods described below. The choice of codons, suitable expression vectors and suitable host cells will vary depending on a number of factors, and can be easily optimized as needed.

### Multiplex Nucleic Acid Assembly

In aspects of the invention, multiplex nucleic acid assembly relates to the assembly of a plurality of nucleic acids to generate a longer nucleic acid product. In one aspect, multiplex oligonucleotide assembly relates to the assembly of a plurality of oligonucleotides to generate a longer nucleic acid molecule. However, it should be appreciated that other nucleic acids (e.g., single or double-stranded nucleic acid degradation products, restriction fragments, amplification products, naturally occurring small nucleic acids, other polynucleotides, etc.) may be assembled or included in a multiplex assembly reaction (e.g., along with one or more oligonucleotides) in order to generate an assembled nucleic acid molecule that is longer than any of the single starting nucleic acids (e.g., oligonucleotides) that were added to the assembly reaction. In certain embodiments, one or more nucleic acid fragments that each were assembled in separate multiplex assembly reactions (e.g., separate multiplex oligonucleotide assembly reactions) may be combined and assembled to form a further nucleic acid that is longer than any of the input nucleic acid fragments. In certain embodiments, one or more nucleic acid fragments that each were assembled in separate multiplex assembly reactions (e.g., separate multiplex oligonucleotide assembly reactions) may be combined with one or more additional nucleic acids (e.g., single or double-stranded nucleic acid degradation products, restriction fragments, amplification products, naturally occurring small nucleic acids, other polynucleotides, etc.) and assembled to form a further nucleic acid that is longer than any of the input nucleic acids.

In aspects of the invention, one or more multiplex assembly reactions may be used to generate target nucleic acids having predetermined sequences. In one aspect, a target nucleic acid may have a sequence of a naturally occurring gene and/or other naturally occurring nucleic acid (e.g., a naturally occurring coding sequence, regulatory sequence, non-coding sequence, chromosomal structural sequence such as a telomere or centromere sequence, etc., any fragment thereof or any combination of two or more thereof). In another aspect, a target nucleic acid may have a sequence that is not naturally-occurring. In one embodiment, a target nucleic acid may be designed to have a sequence that differs from a natural sequence at one or more positions. In other embodiments, a target nucleic acid may be designed to have an entirely novel sequence. However, it should be appreciated that target nucleic acids may include one or more naturally occurring sequences, non-naturally occurring sequences, or combinations thereof.

In one aspect of the invention, multiplex assembly may be used to generate libraries of nucleic acids having different sequences. In some embodiments, a library may contain nucleic acids having random sequences. In certain embodiments, a predetermined target nucleic acid may be designed and assembled to include one or more random sequences at one or more predetermined positions.

In certain embodiments, a target nucleic acid may include a functional sequence (e.g., a protein binding sequence, a regulatory sequence, a sequence encoding a functional protein, etc., or any combination thereof). However, some embodiments of a target nucleic acid may lack a specific functional sequence (e.g., a target nucleic acid may include only non-functional fragments or variants of a protein binding sequence, regulatory sequence, or protein encoding sequence, or any other non-functional naturally-occurring or synthetic sequence, or any non-functional combination thereof). Certain target nucleic acids may include both functional and non-functional sequences. These and other aspects of target nucleic acids and their uses are described in more detail herein.

A target nucleic acid may be assembled in a single multiplex assembly reaction (e.g., a single oligonucleotide assembly reaction). However, a target nucleic acid also may be assembled from a plurality of nucleic acid fragments, each of which may have been generated in a separate multiplex oligonucleotide assembly reaction. It should be appreciated that one or more nucleic acid fragments generated via multiplex oligonucleotide assembly also may be combined with one or more nucleic acid molecules obtained from another source (e.g., a restriction fragment, a nucleic acid amplification product, etc.) to form a target nucleic acid. In some embodiments, a target nucleic acid that is assembled in a first reaction may be used as an input nucleic acid fragment for a subsequent assembly reaction to produce a larger target nucleic acid.

Accordingly, different strategies may be used to produce a target nucleic acid having a predetermined sequence. For example, different starting nucleic acids (e.g., different sets of predetermined nucleic acids) may be assembled to produce the same predetermined target nucleic acid sequence. Also, predetermined nucleic acid fragments may be assembled using one or more different in vitro and/or in vivo techniques. For example, nucleic acids (e.g., overlapping nucleic acid fragments) may be assembled in an in vitro reaction using an enzyme (e.g., a ligase and/or a polymerase) or a chemical reaction (e.g., a chemical ligation) or in vivo (e.g., assembled in a host cell after transfection into the host cell), or a combination thereof. Similarly, each nucleic acid fragment that is used to make a target nucleic acid may be assembled from different sets of oligonucleotides. Also, a nucleic acid fragment may be assembled using an in vitro or an in vivo technique (e.g., an in vitro or in vivo polymerase, recombinase, and/or ligase based assembly process). In addition, different in vitro assembly reactions may be used to produce a nucleic acid fragment. For example, an in vitro oligonucleotide assembly reaction may involve one or more polymerases, ligases, other suitable enzymes, chemical reactions, or any combination thereof.

According to one embodiment, a predetermined nucleic acid fragment may be assembled from a plurality of different starting nucleic acids (e.g., oligonucleotides) in a multiplex assembly reaction (e.g., a multiplex enzyme-mediated reaction, a multiplex chemical assembly reaction, or a combination thereof). Certain aspects of multiplex nucleic acid assembly reactions are illustrated by the following description of certain embodiments of multiplex oligonucleotide assembly reactions. It should be appreciated that the description of the assembly reactions in the context of oligonucleotides is not intended to be limiting. The assembly reactions described herein may be performed using starting nucleic acids obtained from one or more different sources (e.g., synthetic or natural polynucleotides, nucleic acid amplification products, nucleic acid degradation products, oligonucleotides, etc.). The starting nucleic acids may be referred to as assembly nucleic acids (e.g., assembly oligonucleotides). As used herein, an assembly nucleic acid has a sequence that is designed to be incorporated into the nucleic acid product generated during the assembly process. However, it should be appreciated that the description of the assembly reactions in the context of single-stranded nucleic acids is not intended to be limiting. In some embodiments, one or more of the starting nucleic acids illustrated in the figures and described herein may be provided as double stranded nucleic acids. Accordingly, it should be appreciated that where the figures and description illustrate the assembly of single-stranded nucleic acids, the presence of one or more complementary nucleic acids is contemplated. It should be appreciated that the reference to complementary nucleic acids or complementary nucleic acid regions herein refers to nucleic acids or regions thereof that have sequences which are reverse complements of each other so that they can hybridize in an antiparallel fashion typical of natural DNA. Accordingly, one or more double-stranded complementary nucleic acids may be included in a reaction that is described herein in the context of a single-stranded assembly nucleic acid. However, in some embodiments the presence of one or more complementary nucleic acids may interfere with an assembly reaction by competing for hybridization with one of the input assembly nucleic acids. Accordingly, in some embodiments an assembly reaction may involve only single-stranded assembly nucleic acids (i.e., the assembly nucleic acids may be provided in a single-stranded form without their complementary strand) as described or illustrated herein. However, in certain embodiments the presence of one or more complementary nucleic acids may have no or little effect on the assembly reaction. In some embodiments, complementary nucleic acid(s) may be incorporated during one or more steps of an assembly. In yet further embodiments, assembly nucleic acids and their complementary strands may be assembled under the same assembly conditions via parallel assembly reactions in the same reaction mixture. In certain embodiments, a nucleic acid product resulting from the assembly of a plurality of starting nucleic acids may be identical to the nucleic acid product that results from the assembly of nucleic acids that are complementary to the starting nucleic acids (e.g., in some embodiments where the assembly steps result in the production of a double-stranded nucleic acid product). As used herein, an oligonucleotide may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 100 nucleotides long (e.g., from about 30 to 90, 40 to 85, 50 to 80, 60 to 75, or about 65 or about 70 nucleotides long), between about 100 and about 200, between about 200 and about 300 nucleotides, between about 300 and about 400, or between about 400 and about 500 nucleotides long. However, shorter or longer oligonucleotides may be used. An oligonucleotide may be a single-stranded nucleic acid. However, in some embodiments a double-stranded oligonucleotide may be used as described herein. In certain embodiments, an oligonucleotide may be chemically synthesized as described in more detail below.

In some embodiments, an input nucleic acid (e.g., oligonucleotide) may be amplified before use. The resulting product may be double-stranded. In some embodiments, one of the strands of a double-stranded nucleic acid may be removed before use so that only a predetermined single strand is added to an assembly reaction.

In certain embodiments, each oligonucleotide may be designed to have a sequence that is identical to a different portion of the sequence of a predetermined target nucleic acid that is to be assembled. Accordingly, in some embodiments each oligonucleotide may have a sequence that is identical to a portion of one of the two strands of a double-stranded target nucleic acid. For clarity, the two complementary strands of a double stranded nucleic acid are referred to herein as the positive (P) and negative (N) strands. This designation is not intended to imply that the strands are sense and anti-sense strands of a coding sequence. They refer only to the two complementary strands of a nucleic acid (e.g., a target nucleic acid, an intermediate nucleic acid fragment, etc.) regardless of the sequence or function of the nucleic acid. Accordingly, in some embodiments a P strand may be a sense strand of a coding sequence, whereas in other embodiments a P strand may be an anti-sense strand of a coding sequence. According to the invention, a target nucleic acid may be either the P strand, the N strand, or a double-stranded nucleic acid comprising both the P and N strands.

It should be appreciated that different oligonucleotides may be designed to have different lengths. In some embodiments, one or more different oligonucleotides may have overlapping sequence regions (e.g., overlapping 5′ regions or overlapping 3′ regions). Overlapping sequence regions may be identical (i.e., corresponding to the same strand of the nucleic acid fragment) or complementary (i.e., corresponding to complementary strands of the nucleic acid fragment). The plurality of oligonucleotides may include one or more oligonucleotide pairs with overlapping identical sequence regions, one or more oligonucleotide pairs with overlapping complementary sequence regions, or a combination thereof. Overlapping sequences may be of any suitable length. For example, overlapping sequences may encompass the entire length of one or more nucleic acids used in an assembly reaction. Overlapping sequences may be between about 5 and about 500 nucleotides long (e.g., between about 10 and 100, between about 10 and 75, between about 10 and 50, about 20, about 25, about 30, about 35, about 40, about 45, about 50, etc.) However, shorter, longer or intermediate overlapping lengths may be used. It should be appreciated that overlaps between different input nucleic acids used in an assembly reaction may have different lengths.

In a multiplex oligonucleotide assembly reaction designed to generate a predetermined nucleic acid fragment, the combined sequences of the different oligonucleotides in the reaction may span the sequence of the entire nucleic acid fragment on either the positive strand, the negative strand, both strands, or a combination of portions of the positive strand and portions of the negative strand. The plurality of different oligonucleotides may provide either positive sequences, negative sequences, or a combination of both positive and negative sequences corresponding to the entire sequence of the nucleic acid fragment to be assembled. In some embodiments, the plurality of oligonucleotides may include one or more oligonucleotides having sequences identical to one or more portions of the positive sequence, and one or more oligonucleotides having sequences that are identical to one or more portions of the negative sequence of the nucleic acid fragment. One or more pairs of different oligonucleotides may include sequences that are identical to overlapping portions of the predetermined nucleic acid fragment sequence as described herein (e.g., overlapping sequence portions from the same or from complementary strands of the nucleic acid fragment). In some embodiments, the plurality of oligonucleotides includes a set of oligonucleotides having sequences that combine to span the entire positive sequence and a set oligonucleotides having sequences that combine to span the entire negative sequence of the predetermined nucleic acid fragment. However, in certain embodiments, the plurality of oligonucleotides may include one or more oligonucleotides with sequences that are identical to sequence portions on one strand (either the positive or negative strand) of the nucleic acid fragment, but no oligonucleotides with sequences that are complementary to those sequence portions. In one embodiment, a plurality of oligonucleotides includes only oligonucleotides having sequences identical to portions of the positive sequence of the predetermined nucleic acid fragment. In one embodiment, a plurality of oligonucleotides includes only oligonucleotides having sequences identical to portions of the negative sequence of the predetermined nucleic acid fragment. These oligonucleotides may be assembled by sequential ligation or in an extension-based reaction (e.g., if an oligonucleotide having a 3′ region that is complementary to one of the plurality of oligonucleotides is added to the reaction).

In one aspect, a nucleic acid fragment may be assembled in a polymerase-mediated assembly reaction from a plurality of oligonucleotides that are combined and extended in one or more rounds of polymerase-mediated extensions. In another aspect, a nucleic acid fragment may be assembled in a ligase-mediated reaction from a plurality of oligonucleotides that are combined and ligated in one or more rounds of ligase-mediated ligations. In another aspect, a nucleic acid fragment may be assembled in a non-enzymatic reaction (e.g., a chemical reaction) from a plurality of oligonucleotides that are combined and assembled in one or more rounds of non-enzymatic reactions. In some embodiments, a nucleic acid fragment may be assembled using a combination of polymerase, ligase, and/or non-enzymatic reactions. For example, both polymerase(s) and ligase(s) may be included in an assembly reaction mixture. Accordingly, a nucleic acid may be assembled via coupled amplification and ligation or ligation during amplification. The resulting nucleic acid fragment from each assembly technique may have a sequence that includes the sequences of each of the plurality of assembly oligonucleotides that were used as described herein. These assembly reactions may be referred to as primerless assemblies, since the target nucleic acid is generated by assembling the input oligonucleotides rather than being generated in an amplification reaction where the oligonucleotides act as amplification primers to amplify a pre-existing template nucleic acid molecule corresponding to the target nucleic acid.

Polymerase-based assembly techniques may involve one or more suitable polymerase enzymes that can catalyze a template-based extension of a nucleic acid in a 5′ to 3′ direction in the presence of suitable nucleotides and an annealed template. A polymerase may be thermostable. A polymerase may be obtained from recombinant or natural sources. In some embodiments, a thermostable polymerase from a thermophilic organism may be used. In some embodiments, a polymerase may include a 3′→5′ exonuclease/proofreading activity. In some embodiments, a polymerase may have no, or little, proofreading activity (e.g., a polymerase may be a recombinant variant of a natural polymerase that has been modified to reduce its proofreading activity). Examples of thermostable DNA polymerases include, but are not limited to: Taq (a heat-stable DNA polymerase from the bacterium Thermus aquaticus); Pfu (a thermophilic DNA polymerase with a 3′→5′ exonuclease/proofreading activity from Pyrococcus furiosus, available from for example Promega); VentR® DNA Polymerase and VentR® (exo-) DNA Polymerase (thermophilic DNA polymerases with or without a 3′→5′ exonuclease/proofreading activity from Thermococcus litoralis; also known as Tli polymerase); Deep VentR® DNA Polymerase and Deep VentR® (exo-) DNA Polymerase (thermophilic DNA polymerases with or without a 3′→5′ exonuclease/proofreading activity from Pyrococcus species GB-D; available from New England Biolabs); KOD HiFi (a recombinant Thermococcus kodakaraensis KOD1 DNA polymerase with a 3′→5′ exonuclease/proofreading activity, available from Novagen,); BIO-X-ACT (a mix of polymerases that possesses 5′→3′ DNA polymerase activity and 3′→5′ proofreading activity); Klenow Fragment (an N-terminal truncation of E. coli DNA Polymerase I which retains polymerase activity, but has lost the 5′→3′ exonuclease activity, available from, for example, Promega and NEB); Sequenase™ (T7 DNA polymerase deficient in 3′-5′ exonuclease activity); Phi29 (bacteriophage 29 DNA polymerase, may be used for rolling circle amplification, for example, in a TempliPhi™ DNA Sequencing Template Amplification Kit, available from Amersham Biosciences); TopoTaq™ (a hybrid polymerase that combines hyperstable DNA binding domains and the DNA unlinking activity of Methanopyrus topoisomerase, with no exonuclease activity, available from Fidelity Systems); TopoTaq HiFi which incorporates a proofreading domain with exonuclease activity; Phusion™ (a Pyrococcus-like enzyme with a processivity-enhancing domain, available from New England Biolabs); any other suitable DNA polymerase, or any combination of two or more thereof.

Ligase-based assembly techniques may involve one or more suitable ligase enzymes that can catalyze the covalent linking of adjacent 3′ and 5′ nucleic acid termini (e.g., a 5′ phosphate and a 3′ hydroxyl of nucleic acid(s) annealed on a complementary template nucleic acid such that the 3′ terminus is immediately adjacent to the 5′ terminus). Accordingly, a ligase may catalyze a ligation reaction between the 5′ phosphate of a first nucleic acid to the 3′ hydroxyl of a second nucleic acid if the first and second nucleic acids are annealed next to each other on a template nucleic acid). A ligase may be obtained from recombinant or natural sources. A ligase may be a heat-stable ligase. In some embodiments, a thermostable ligase from a thermophilic organism may be used. Examples of thermostable DNA ligases include, but are not limited to: Tth DNA ligase (from Thermus thermophilus, available from, for example, Eurogentec and GeneCraft); Pfu DNA ligase (a hyperthermophilic ligase from Pyrococcus furiosus); Taq ligase (from Thermus aquaticus), any other suitable heat-stable ligase, or any combination thereof. In some embodiments, one or more lower temperature ligases may be used (e.g., T4 DNA ligase). A lower temperature ligase may be useful for shorter overhangs (e.g., about 3, about 4, about 5, or about 6 base overhangs) that may not be stable at higher temperatures.

Non-enzymatic techniques can be used to ligate nucleic acids. For example, a 5′-end (e.g., the 5′ phosphate group) and a 3′-end (e.g., the 3′ hydroxyl) of one or more nucleic acids may be covalently linked together without using enzymes (e.g., without using a ligase). In some embodiments, non-enzymatic techniques may offer certain advantages over enzyme-based ligations. For example, non-enzymatic techniques may have a high tolerance of non-natural nucleotide analogues in nucleic acid substrates, may be used to ligate short nucleic acid substrates, may be used to ligate RNA substrates, and/or may be cheaper and/or more suited to certain automated (e.g., high throughput) applications.

Non-enzymatic ligation may involve a chemical ligation. In some embodiments, nucleic acid termini of two or more different nucleic acids may be chemically ligated. In some embodiments, nucleic acid termini of a single nucleic acid may be chemically ligated (e.g., to circularize the nucleic acid). It should be appreciated that both strands at a first double-stranded nucleic acid terminus may be chemically ligated to both strands at a second double-stranded nucleic acid terminus. However, in some embodiments only one strand of a first nucleic acid terminus may be chemically ligated to a single strand of a second nucleic acid terminus. For example, the 5′ end of one strand of a first nucleic acid terminus may be ligated to the 3′ end of one strand of a second nucleic acid terminus without the ends of the complementary strands being chemically ligated.

Accordingly, a chemical ligation may be used to form a covalent linkage between a 5′ terminus of a first nucleic acid end and a 3′ terminus of a second nucleic acid end, wherein the first and second nucleic acid ends may be ends of a single nucleic acid or ends of separate nucleic acids. In one aspect, chemical ligation may involve at least one nucleic acid substrate having a modified end (e.g., a modified 5′ and/or 3′ terminus) including one or more chemically reactive moieties that facilitate or promote linkage formation. In some embodiments, chemical ligation occurs when one or more nucleic acid termini are brought together in close proximity (e.g., when the termini are brought together due to annealing between complementary nucleic acid sequences). Accordingly, annealing between complementary 3′ or 5′ overhangs (e.g., overhangs generated by restriction enzyme cleavage of a double-stranded nucleic acid) or between any combination of complementary nucleic acids that results in a 3′ terminus being brought into close proximity with a 5′ terminus (e.g., the 3′ and 5′ termini are adjacent to each other when the nucleic acids are annealed to a complementary template nucleic acid) may promote a template-directed chemical ligation. Examples of chemical reactions may include, but are not limited to, condensation, reduction, and/or photo-chemical ligation reactions. It should be appreciated that in some embodiments chemical ligation can be used to produce naturally-occurring phosphodiester internucleotide linkages, non-naturally-occurring phosphamide pyrophosphate internucleotide linkages, and/or other non-naturally-occurring internucleotide linkages.

In some embodiments, the process of chemical ligation may involve one or more coupling agents to catalyze the ligation reaction. A coupling agent may promote a ligation reaction between reactive groups in adjacent nucleic acids (e.g., between a 5′-reactive moiety and a 3′-reactive moiety at adjacent sites along a complementary template). In some embodiments, a coupling agent may be a reducing reagent (e.g., ferricyanide), a condensing reagent such (e.g., cyanoimidazole, cyanogen bromide, carbodiimide, etc.), or irradiation (e.g., UV irradiation for photo-ligation).

In some embodiments, a chemical ligation may be an autoligation reaction that does not involve a separate coupling agent. In autoligation, the presence of a reactive group on one or more nucleic acids may be sufficient to catalyze a chemical ligation between nucleic acid termini without the addition of a coupling agent (see, for example, Xu Y & Kool E T, 1997, Tetrahedron Lett. 38:5595-8). Non-limiting examples of these reagent-free ligation reactions may involve nucleophilic displacements of sulfur on bromoacetyl, tosyl, or iodo-nucleoside groups (see, for example, Xu Y et al., 2001, Nat Biotech 19:148-52). Nucleic acids containing reactive groups suitable for autoligation can be prepared directly on automated synthesizers (see, for example, Xu Y & Kool E T, 1999, Nuc. Acids Res. 27:875-81). In some embodiments, a phosphorothioate at a 3′ terminus may react with a leaving group (such as tosylate or iodide) on a thymidine at an adjacent 5′ terminus. In some embodiments, two nucleic acid strands bound at adjacent sites on a complementary target strand may undergo auto-ligation by displacement of a 5′-end iodide moiety (or tosylate) with a 3′-end sulfur moiety. Accordingly, in some embodiments the product of an autoligation may include a non-naturally-occurring internucleotide linkage (e.g., a single oxygen atom may be replaced with a sulfur atom in the ligated product).

In some embodiments, a synthetic nucleic acid duplex can be assembled via chemical ligation in a one step reaction involving simultaneous chemical ligation of nucleic acids on both strands of the duplex. For example, a mixture of 5′-phosphorylated oligonucleotides corresponding to both strands of a target nucleic acid may be chemically ligated by a) exposure to heat (e.g., to 97° C.) and slow cooling to form a complex of annealed oligonucleotides, and b) exposure to cyanogen bromide or any other suitable coupling agent under conditions sufficient to chemically ligate adjacent 3′ and 5′ ends in the nucleic acid complex.

In some embodiments, a synthetic nucleic acid duplex can be assembled via chemical ligation in a two step reaction involving separate chemical ligations for the complementary strands of the duplex. For example, each strand of a target nucleic acid may be ligated in a separate reaction containing phosphorylated oligonucleotides corresponding to the strand that is to be ligated and non-phosphorylated oligonucleotides corresponding to the complementary strand. The non-phosphorylated oligonucleotides may serve as a template for the phosphorylated oligonucleotides during a chemical ligation (e.g. using cyanogen bromide). The resulting single-stranded ligated nucleic acid may be purified and annealed to a complementary ligated single-stranded nucleic acid to form the target duplex nucleic acid (see, for example, Shabarova Z A et al., 1991, Nuc. Acids Res. 19:4247-51).

Aspects of the invention may be used to enhance different types of nucleic acid assembly reactions (e.g., multiplex nucleic acid assembly reactions). Aspects of the invention may be used in combination with one or more assembly reactions described in, for example, Carr et al., 2004, Nucleic Acids Research, Vol. 32, No 20, e 162 (9 pages); Richmond et al., 2004, Nucleic Acids Research, Vol. 32, No 17, pp 5011-5018; Caruthers et al., 1972, J. Mol. Biol. 72, 475-492; Hecker et al., 1998, Biotechniques 24:256-260; Kodumal et al., 2004, PNAS Vol. 101, No. 44, pp 15573-15578; Tian et al., 2004, Nature, Vol. 432, pp 1050-1054; and U.S. Pat. Nos. 6,008,031 and 5,922,539, the disclosures of which are incorporated herein by reference. Certain embodiments of multiplex nucleic acid assembly reactions for generating a predetermined nucleic acid fragment are illustrated with reference to FIGS. 1-4. It should be appreciated that multiplex nucleic acid assembly reactions may be performed in any suitable format, including in a reaction tube, in a multi-well plate, on a surface, on a column, in a microfluidic device (e.g., a microfludic tube), a capillary tube, etc.

FIG. 1 shows one embodiment of a plurality of oligonucleotides that may be assembled in a polymerase-based multiplex oligonucleotide assembly reaction. FIG. 1A shows two groups of oligonucleotides (Group P and Group N) that have sequences of portions of the two complementary strands of a nucleic acid fragment to be assembled. Group P includes oligonucleotides with positive strand sequences (P₁, P₂, . . . Pₙ₋₁, Pₙ, Pₙ₊₁, . . . P_{T}, shown from 5′→3′ on the positive strand). Group N includes oligonucleotides with negative strand sequences (N_{T}, . . . , Nₙ₊₁, Nₙ, Nₙ₋₁, . . . , N₂, N₁, shown from 5′→3′ on the negative strand). In this example, none of the P group oligonucleotides overlap with each other and none of the N group oligonucleotides overlap with each other. However, in some embodiments, one or more of the oligonucleotides within the S or N group may overlap. Furthermore, FIG. 1A shows gaps between consecutive oligonucleotides in Group P and gaps between consecutive oligonucleotides in Group N. However, each P group oligonucleotide (except for P₁) and each N group oligonucleotide (except for N_{T}) overlaps with complementary regions of two oligonucleotides from the complementary group of oligonucleotides. P₁ and N_{T} overlap with a complementary region of only one oligonucleotide from the other group (the complementary 3′-most oligonucleotides N₁ and P_{T}, respectively). FIG. 1B shows a structure of an embodiment of a Group P or Group N oligonucleotide represented in FIG. 1A. This oligonucleotide includes a 5′ region that is complementary to a 5′ region of a first oligonucleotide from the other group, a 3′ region that is complementary to a 3′ region of a second oligonucleotide from the other group, and a core or central region that is not complementary to any oligonucleotide sequence from the other group (or its own group). This central region is illustrated as the B region in FIG. 1B. The sequence of the B region may be different for each different oligonucleotide. As defined herein, the B region of an oligonucleotide in one group corresponds to a gap between two consecutive oligonucleotides in the complementary group of oligonucleotides. It should be noted that the 5′-most oligonucleotide in each group (P₁ in Group P and N_{T} in Group N) does not have a 5′ region that is complementary to the 5′ region of any other oligonucleotide in either group. Accordingly, the 5′-most oligonucleotides (P₁ and N_{T}) that are illustrated in FIG. 1A each have a 3′ complementary region and a 5′ non-complementary region (the B region of FIG. 1B), but no 5′ complementary region. However, it should be appreciated that any one or more of the oligonucleotides in Group P and/or Group N (including all of the oligonucleotides in Group P and/or Group N) can be designed to have no B region. In the absence of a B region, a 5′-most oligonucleotide has only the 3′ complementary region (meaning that the entire oligonucleotide is complementary to the 3′ region of the 3′-most oligonucleotide from the other group (e.g., the 3′ region of N₁ or P_{T} shown in FIG. 1A). In the absence of a B region, one of the other oligonucleotides in either Group P or Group N has only a 5′ complementary region and a 3′ complementary region (meaning that the entire oligonucleotide is complementary to the 5′ and 3′ sequence regions of the two overlapping oligonucleotides from the complementary group). In some embodiments, only a subset of oligonucleotides in an assembly reaction may include B regions. It should be appreciated that the length of the 5′, 3′, and B regions may be different for each oligonucleotide. However, for each oligonucleotide the length of the 5′ region is the same as the length of the complementary 5′ region in the 5′ overlapping oligonucleotide from the other group. Similarly, the length of the 3′ region is the same as the length of the complementary 3′ region in the 3′ overlapping oligonucleotide from the other group. However, in certain embodiments a 3′-most oligonucleotide may be designed with a 3′ region that extends beyond the 5′ region of the 5′-most oligonucleotide. In this embodiment, an assembled product may include the 5′ end of the 5′-most oligonucleotide, but not the 3′ end of the 3′-most oligonucleotide that extends beyond it.

FIG. 1C illustrates a subset of the oligonucleotides from FIG. 1A, each oligonucleotide having a 5′, a 3′, and an optional B region. Oligonucleotide Pₙ is shown with a 5′ region that is complementary to (and can anneal to) the 5′ region of oligonucleotide Nₙ₋₁. Oligonucleotide Pₙ also has a 3′ region that is complementary to (and can anneal to) the 3′ region of oligonucleotide Nₙ. Nₙ is also shown with a 5′ region that is complementary (and can anneal to) the 5′ region of oligonucleotide Pₙ₊₁. This pattern could be repeated for all of oligonucleotides P₂ to P_{T} and N₁ to N_{T-1} (with the 5′-most oligonucleotides only having 3′ complementary regions as discussed herein). If all of the oligonucleotides from Group P and Group N are mixed together under appropriate hybridization conditions, they may anneal to form a long chain such as the oligonucleotide complex illustrated in FIG. 1A. However, subsets of the oligonucleotides may form shorter chains and even oligonucleotide dimers with annealed 5′ or 3′ regions. It should be appreciated that many copies of each oligonucleotide are included in a typical reaction mixture. Accordingly, the resulting hybridized reaction mixture may contain a distribution of different oligonucleotide dimers and complexes. Polymerase-mediated extension of the hybridized oligonucleotides results in a template-based extension of the 3′ ends of oligonucleotides that have annealed 3′ regions. Accordingly, polymerase-mediated extension of the oligonucleotides shown in FIG. 1C would result in extension of the 3′ ends only of oligonucleotides Pₙ and Nₙ generating extended oligonucleotides containing sequences that are complementary to all the regions of Nₙ and Pₙ, respectively. Extended oligonucleotide products with sequences complementary to all of Nₙ₋₁ and Pₙ₊₁ would not be generated unless oligonucleotides Pₙ₋₁ and Nₙ₊₁ were included in the reaction mixture. Accordingly, if all of the oligonucleotide sequences in a plurality of oligonucleotides are to be incorporated into an assembled nucleic acid fragment using a polymerase, the plurality of oligonucleotides should include 5′-most oligonucleotides that are at least complementary to the entire 3′ regions of the 3′-most oligonucleotides. In some embodiments, the 5′-most oligonucleotides also may have 5′ regions that extend beyond the 3′ ends of the 3′-most oligonucleotides as illustrated in FIG. 1A. In some embodiments, a ligase also may be added to ligate adjacent 5′ and 3′ ends that may be formed upon 3′ extension of annealed oligonucleotides in an oligonucleotide complex such as the one illustrated in FIG. 1A.

When assembling a nucleic acid fragment using a polymerase, a single cycle of polymerase extension extends oligonucleotide pairs with annealed 3′ regions. Accordingly, if a plurality of oligonucleotides were annealed to form an annealed complex such as the one illustrated in FIG. 1A, a single cycle of polymerase extension would result in the extension of the 3′ ends of the P₁/N₁, P₂/N₂, . . . , Pₙ₋₁/Nₙ₋₁, Pₙ/Nₙ, Pₙ₊₁/N₁₊₁, . . . , P_{T}/N_{T} oligonucleotide pairs. In one embodiment, a single molecule could be generated by ligating the extended oligonucleotide dimers. In one embodiment, a single molecule incorporating all of the oligonucleotide sequences may be generated by performing several polymerase extension cycles.

In one embodiment, FIG. 1D illustrates two cycles of polymerase extension (separated by a denaturing step and an annealing step) and the resulting nucleic acid products. It should be appreciated that several cycles of polymerase extension may be required to assemble a single nucleic acid fragment containing all the sequences of an initial plurality of oligonucleotides. In one embodiment, a minimal number of extension cycles for assembling a nucleic acid may be calculated as log₂n, where n is the number of oligonucleotides being assembled. In some embodiments, progressive assembly of the nucleic acid may be achieved without using temperature cycles. For example, an enzyme capable of rolling circle amplification may be used (e.g., phi 29 polymerase) when a circularized nucleic acid (e.g., oligonucleotide) complex is used as a template to produce a large amount of circular product for subsequent processing using MutS or a MutS homolog as described herein. In step 1 of FIG. 1D, annealed oligonucleotide pairs Pₙ/Nₙ and Pₙ₊₁/Nₙ₊₁ are extended to form oligonucleotide dimer products incorporating the sequences covered by the respective oligonucleotide pairs. For example, Pₙ is extended to incorporate sequences that are complementary to the B and 5′ regions of Nₙ (indicated as N′ₙ in FIG. 1D). Similarly, Nₙ₊₁ is extended to incorporate sequences that are complementary to the 5′ and B regions of Pₙ₊₁ (indicated as P′ₙ₊₁ in FIG. 1D). These dimer products may be denatured and reannealed to form the starting material of step 2 where the 3′ end of the extended Pₙ oligonucleotide is annealed to the 3′ end of the extended Nₙ₊₁ oligonucleotide. This product may be extended in a polymerase-mediated reaction to form a product that incorporates the sequences of the four oligonucleotides (Pₙ, Nₙ, Pₙ+1, Nₙ₊₁). One strand of this extended product has a sequence that includes (in 5′ to 3′ order) the 5′, B, and 3′ regions of Pₙ, the complement of the B region of Nₙ, the 5′, B, and 3′ regions of Pₙ₊₁, and the complements of the B and 5′ regions of Nₙ₊₁. The other strand of this extended product has the complementary sequence. It should be appreciated that the 3′ regions of Pₙ and Nₙ are complementary, the 5′ regions of Nₙ and Pₙ₊₁ are complementary, and the 3′ regions of Pₙ₊₁ and Nₙ₊₁ are complementary. It also should be appreciated that the reaction products shown in FIG. 1D are a subset of the reaction products that would be obtained using all of the oligonucleotides of Group P and Group N. A first polymerase extension reaction using all of the oligonucleotides would result in a plurality of overlapping oligonucleotide dimers from P₁/N₁ to P_{T}/N_{T}. Each of these may be denatured and at least one of the strands could then anneal to an overlapping complementary strand from an adjacent (either 3′ or 5′) oligonucleotide dimer and be extended in a second cycle of polymerase extension as shown in FIG. 1D. Subsequent cycles of denaturing, annealing, and extension produce progressively larger products including a nucleic acid fragment that includes the sequences of all of the initial oligonucleotides. It should be appreciated that these subsequent rounds of extension also produce many nucleic acid products of intermediate length. The reaction product may be complex since not all of the 3′ regions may be extended in each cycle. Accordingly, unextended oligonucleotides may be available in each cycle to anneal to other unextended oligonucleotides or to previously extended oligonucleotides. Similarly, extended products of different sizes may anneal to each other in each cycle. Accordingly, a mixture of extended products of different sizes covering different regions of the sequence may be generated along with the nucleic acid fragment covering the entire sequence. This mixture also may contain any remaining unextended oligonucleotides.

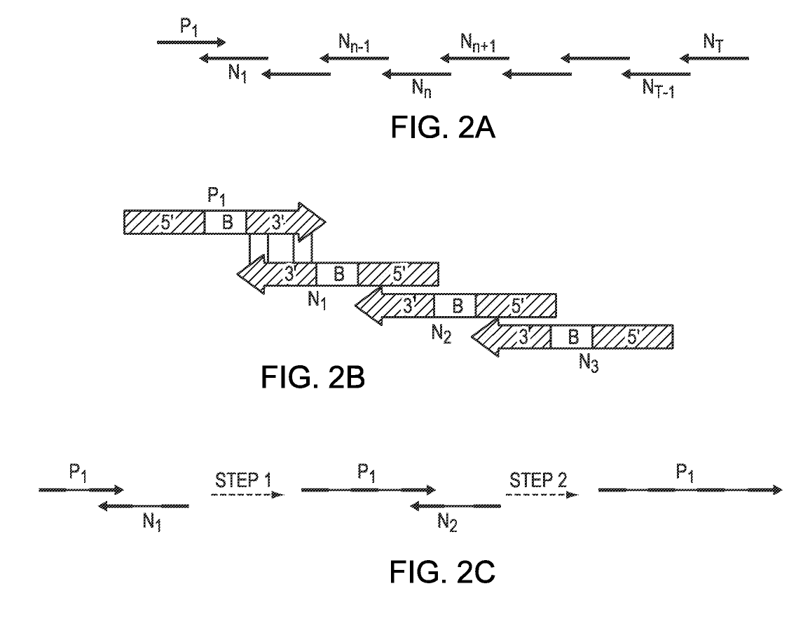

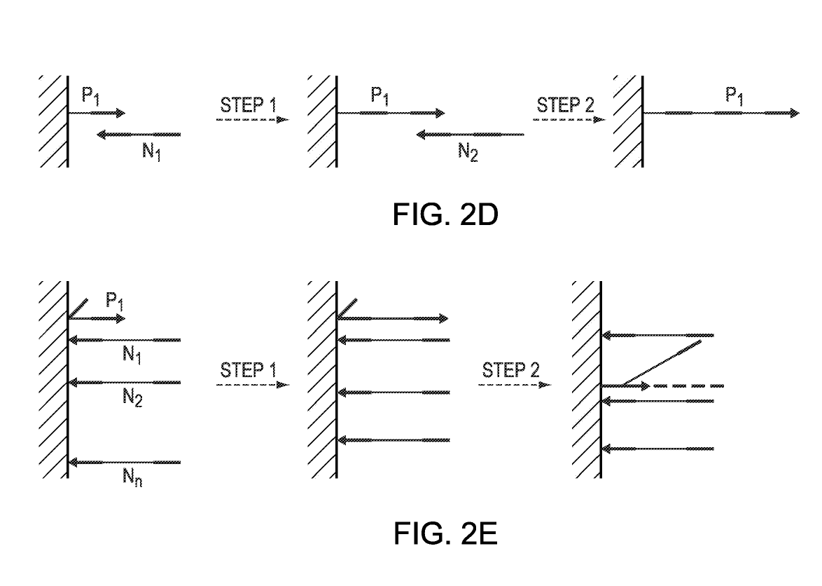

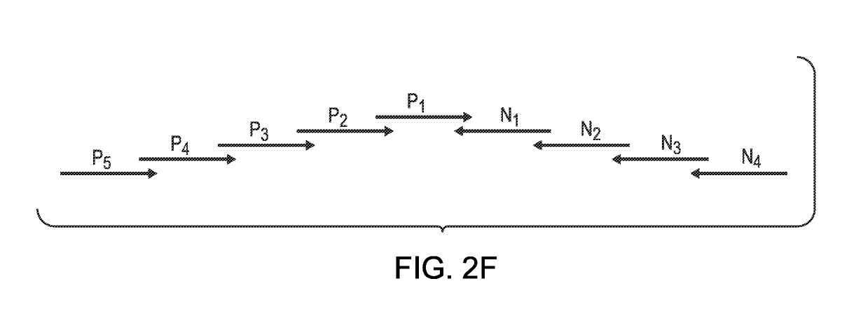

FIG. 2 shows an embodiment of a plurality of oligonucleotides that may be assembled in a directional polymerase-based multiplex oligonucleotide assembly reaction. In this embodiment, only the 5′-most oligonucleotide of Group P may be provided. In contrast to the example shown in FIG. 1, the remainder of the sequence of the predetermined nucleic acid fragment is provided by oligonucleotides of Group N. The 3′-most oligonucleotide of Group N(N1) has a 3′ region that is complementary to the 3′ region of P₁ as shown in FIG. 2B. However, the remainder of the oligonucleotides in Group N have overlapping (but non-complementary) 3′ and 5′ regions as illustrated in FIG. 2B for oligonucleotides N1-N3. Each Group N oligonucleotide (e.g., Nₙ) overlaps with two adjacent oligonucleotides: one overlaps with the 3′ region (Nₙ₋₁) and one with the 5′ region (Nₙ₊₁), except for N₁ that overlaps with the 3′ regions of P₁ (complementary overlap) and N2 (non-complementary overlap), and NT that overlaps only with N_{T-1}. It should be appreciated that all of the overlaps shown in FIG. 2A between adjacent oligonucleotides N₂ to N_{T-1} are non-complementary overlaps between the 5′ region of one oligonucleotide and the 3′ region of the adjacent oligonucleotide illustrated in a 3′ to 5′ direction on the N strand of the predetermined nucleic acid fragment. It also should be appreciated that each oligonucleotide may have 3′, B, and 5′ regions of different lengths (including no B region in some embodiments). In some embodiments, none of the oligonucleotides may have B regions, meaning that the entire sequence of each oligonucleotide may overlap with the combined 5′ and 3′ region sequences of its two adjacent oligonucleotides.

Assembly of a predetermined nucleic acid fragment from the plurality of oligonucleotides shown in FIG. 2A may involve multiple cycles of polymerase-mediated extension. Each extension cycle may be separated by a denaturing and an annealing step. FIG. 2C illustrates the first two steps in this assembly process. In step 1, annealed oligonucleotides P₁ and N₁ are extended to form an oligonucleotide dimer. P₁ is shown with a 5′ region that is non-complementary to the 3′ region of N₁ and extends beyond the 3′ region of N₁ when the oligonucleotides are annealed. However, in some embodiments, P₁ may lack the 5′ non-complementary region and include only sequences that overlap with the 3′ region of N₁. The product of P₁ extension is shown after step 1 containing an extended region that is complementary to the 5′ end of N₁. The single strand illustrated in FIG. 2C may be obtained by denaturing the oligonucleotide dimer that results from the extension of P₁/N₁ in step 1. The product of P₁ extension is shown annealed to the 3′ region of N₂. This annealed complex may be extended in step 2 to generate an extended product that now includes sequences complementary to the B and 5′ regions of N₂. Again, the single strand illustrated in FIG. 2C may be obtained by denaturing the oligonucleotide dimer that results from the extension reaction of step 2. Additional cycles of extension may be performed to further assemble a predetermined nucleic acid fragment. In each cycle, extension results in the addition of sequences complementary to the B and 5′ regions of the next Group N oligonucleotide. Each cycle may include a denaturing and annealing step. However, the extension may occur under the annealing conditions. Accordingly, in one embodiment, cycles of extension may be obtained by alternating between denaturing conditions (e.g., a denaturing temperature) and annealing/extension conditions (e.g., an annealing/extension temperature). In one embodiment, T (the number of group N oligonucleotides) may determine the minimal number of temperature cycles used to assemble the oligonucleotides. However, in some embodiments, progressive extension may be achieved without temperature cycling. For example, an enzyme capable promoting rolling circle amplification may be used (e.g., TempliPhi). It should be appreciated that a reaction mixture containing an assembled predetermined nucleic acid fragment also may contain a distribution of shorter extension products that may result from incomplete extension during one or more of the cycles or may be the result of an P₁/N₁ extension that was initiated after the first cycle.

FIG. 2D illustrates an example of a sequential extension reaction where the 5′-most P₁ oligonucleotide is bound to a support and the Group N oligonucleotides are unbound. The reaction steps are similar to those described for FIG. 2C. However, an extended predetermined nucleic acid fragment will be bound to the support via the 5′-most P₁ oligonucleotide. Accordingly, the complementary strand (the negative strand) may readily be obtained by denaturing the bound fragment and releasing the negative strand. In some embodiments, the attachment to the support may be labile or readily reversed (e.g., using light, a chemical reagent, a pH change, etc.) and the positive strand also may be released. Accordingly, either the positive strand, the negative strand, or the double-stranded product may be obtained. FIG. 2E illustrates an example of a sequential reaction where P₁ is unbound and the Group N oligonucleotides are bound to a support. The reaction steps are similar to those described for FIG. 2C. However, an extended predetermined nucleic acid fragment will be bound to the support via the 5′-most N_{T} oligonucleotide. Accordingly, the complementary strand (the positive strand) may readily be obtained by denaturing the bound fragment and releasing the positive strand. In some embodiments, the attachment to the support may be labile or readily reversed (e.g., using light, a chemical reagent, a pH change, etc.) and the negative strand also may be released. Accordingly, either the positive strand, the negative strand, or the double-stranded product may be obtained.

It should be appreciated that other configurations of oligonucleotides may be used to assemble a nucleic acid via two or more cycles of polymerase-based extension. In many configurations, at least one pair of oligonucleotides have complementary 3′ end regions. FIG. 2F illustrates an example where an oligonucleotide pair with complementary 3′ end regions is flanked on either side by a series of oligonucleotides with overlapping non-complementary sequences. The oligonucleotides illustrated to the right of the complementary pair have overlapping 3′ and 5′ regions (with the 3′ region of one oligonucleotide being identical to the 5′ region of the adjacent oligonucleotide) that corresponding to a sequence of one strand of the target nucleic acid to be assembled. The oligonucleotides illustrated to the left of the complementary pair have overlapping 3′ and 5′ regions (with the 3′ region of one oligonucleotide being identical to the 5′ region of the adjacent oligonucleotide) that correspond to a sequence of the complementary strand of the target nucleic acid. These oligonucleotides may be assembled via sequential polymerase-based extension reactions as described herein (see also, for example, Xiong et al., 2004, Nucleic Acids Research, Vol. 32, No. 12, e98, 10 pages, the disclosure of which is incorporated by reference herein). It should be appreciated that different numbers and/or lengths of oligonucleotides may be used on either side of the complementary pair. Accordingly, the illustration of the complementary pair as the central pair in FIG. 2F is not intended to be limiting as other configuration of a complementary oligonucleotide pair flanked by a different number of non-complementary pairs on either side may be used according to methods of the invention.

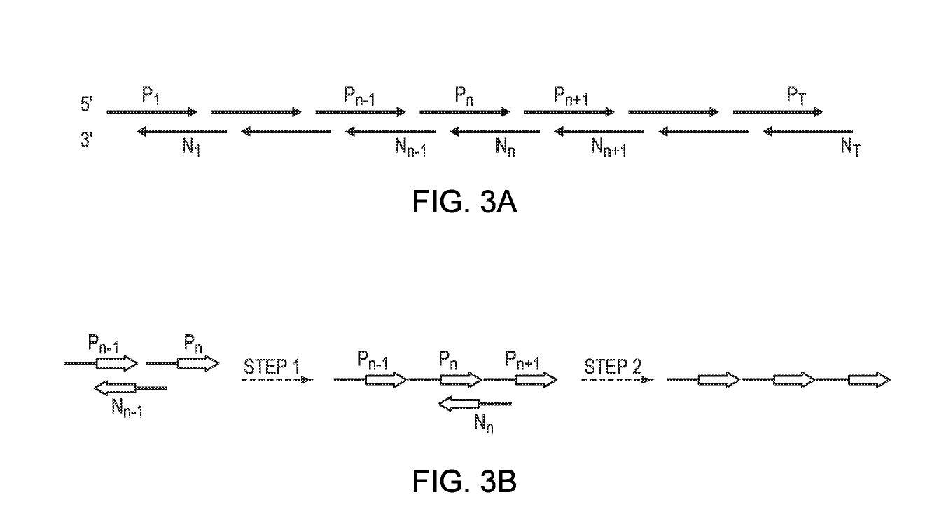

FIG. 3 shows an embodiment of a plurality of oligonucleotides that may be assembled in a ligase reaction. FIG. 3A illustrates the alignment of the oligonucleotides showing that they do not contain gaps (i.e., no B region as described herein). Accordingly, the oligonucleotides may anneal to form a complex with no nucleotide gaps between the 3′ and 5′ ends of the annealed oligonucleotides in either Group P or Group N. These oligonucleotides provide a suitable template for assembly using a ligase under appropriate reaction conditions. However, it should be appreciated that these oligonucleotides also may be assembled using a polymerase-based assembly reaction as described herein. FIG. 3B shows two individual ligation reactions. These reactions are illustrated in two steps. However, it should be appreciated that these ligation reactions may occur simultaneously or sequentially in any order and may occur as such in a reaction maintained under constant reaction conditions (e.g., with no temperature cycling) or in a reaction exposed to several temperature cycles. For example, the reaction illustrated in step 2 may occur before the reaction illustrated in step 1. In each ligation reaction illustrated in FIG. 3B, a Group N oligonucleotide is annealed to two adjacent Group P oligonucleotides (due to the complementary 5′ and 3′ regions between the P and N oligonucleotides), providing a template for ligation of the adjacent P oligonucleotides. Although not illustrated, ligation of the N group oligonucleotides also may proceed in similar manner to assemble adjacent N oligonucleotides that are annealed to their complementary P oligonucleotide. Assembly of the predetermined nucleic acid fragment may be obtained through ligation of all of the oligonucleotides to generate a double stranded product. However, in some embodiments, a single stranded product of either the positive or negative strand may be obtained. In certain embodiments, a plurality of oligonucleotides may be designed to generate only single-stranded reaction products in a ligation reaction. For example, a first group of oligonucleotides (of either Group P or Group N) may be provided to cover the entire sequence on one strand of the predetermined nucleic acid fragment (on either the positive or negative strand). In contrast, a second group of oligonucleotides (from the complementary group to the first group) may be designed to be long enough to anneal to complementary regions in the first group but not long enough to provide adjacent 5′ and 3′ ends between oligonucleotides in the second group. This provides substrates that are suitable for ligation of oligonucleotides from the first group but not the second group. The result is a single-stranded product having a sequence corresponding to the oligonucleotides in the first group. Again, as with other assembly reactions described herein, a ligase reaction mixture that contains an assembled predetermined nucleic acid fragment also may contain a distribution of smaller fragments resulting from the assembly of a subset of the oligonucleotides.

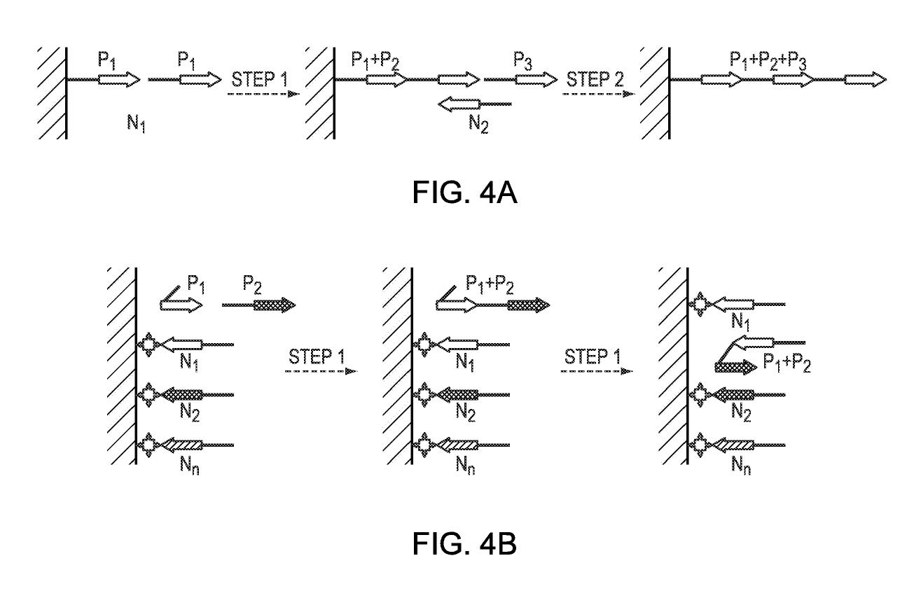

FIG. 4 shows an embodiment of a ligase-based assembly where one or more of the plurality of oligonucleotides is bound to a support. In FIG. 4A, the 5′ most oligonucleotide of the P group oligonucleotides is bound to a support. Ligation of adjacent oligonucleotides in the 5′ to 3′ direction results in the assembly of a predetermined nucleic acid fragment. FIG. 4A illustrates an example where adjacent oligonucleotides P₂ and P₃ are added sequentially. However, the ligation of any two adjacent oligonucleotides from Group P may occur independently and in any order in a ligation reaction mixture. For example, when P₁ is ligated to the 5′ end of N₂, N₂ may be in the form of a single oligonucleotide or it already may be ligated to one or more downstream oligonucleotides (N₃, N₄, etc.). It should be appreciated that for a ligation assembly bound to a support, either the 5′-most (e.g., P₁ for Group P, or N_{T} for Group N) or the 3′-most (e.g., P_{T} for Group P, or N₁ for Group N) oligonucleotide may be bound to a support since the reaction can proceed in any direction. In some embodiments, a predetermined nucleic acid fragment may be assembled with a central oligonucleotide (i.e., neither the 5′-most or the 3′-most) that is bound to a support provided that the attachment to the support does not interfere with ligation.

FIG. 4B illustrates an example where a plurality of N group oligonucleotides are bound to a support and a predetermined nucleic acid fragment is assembled from P group oligonucleotides that anneal to their complementary support-bound N group oligonucleotides. Again, FIG. 4B illustrates a sequential addition. However, adjacent P group oligonucleotides may be ligated in any order. Also, the bound oligonucleotides may be attached at their 5′ end, 3′ end, or at any other position provided that the attachment does not interfere with their ability to bind to complementary 5′ and 3′ regions on the oligonucleotides that are being assembled. This reaction may involve one or more reaction condition changes (e.g., temperature cycles) so that ligated oligonucleotides bound to one immobilized N group oligonucleotide can be dissociated from the support and bind to a different immobilized N group oligonucleotide to provide a substrate for ligation to another P group oligonucleotide.

As with other assembly reactions described herein, support-bound ligase reactions (e.g., those illustrated in FIG. 4B) that generate a full length predetermined nucleic acid fragment also may generate a distribution of smaller fragments resulting from the assembly of subsets of the oligonucleotides. A support used in any of the assembly reactions described herein (e.g., polymerase-based, ligase-based, or other assembly reaction) may include any suitable support medium. A support may be solid, porous, a matrix, a gel, beads, beads in a gel, etc. A support may be of any suitable size. A solid support may be provided in any suitable configuration or shape (e.g., a chip, a bead, a gel, a microfluidic channel, a planar surface, a spherical shape, a column, etc.).

As illustrated herein, different oligonucleotide assembly reactions may be used to assemble a plurality of overlapping oligonucleotides (with overlaps that are either 5′/5′, 3′/3′, 5′/3′, complementary, non-complementary, or a combination thereof). Many of these reactions include at least one pair of oligonucleotides (the pair including one oligonucleotide from a first group or P group of oligonucleotides and one oligonucleotide from a second group or N group of oligonucleotides) have overlapping complementary 3′ regions. However, in some embodiments, a predetermined nucleic acid may be assembled from non-overlapping oligonucleotides using blunt-ended ligation reactions. In some embodiments, the order of assembly of the non-overlapping oligonucleotides may be biased by selective phosphorylation of different 5′ ends. In some embodiments, size purification may be used to select for the correct order of assembly. In some embodiments, the correct order of assembly may be promoted by sequentially adding appropriate oligonucleotide substrates into the reaction (e.g., the ligation reaction).

In order to obtain a full-length nucleic acid fragment from a multiplex oligonucleotide assembly reaction, a purification step may be used to remove starting oligonucleotides and/or incompletely assembled fragments. In some embodiments, a purification step may involve chromatography, electrophoresis, or other physical size separation technique. In certain embodiments, a purification step may involve amplifying the full length product. For example, a pair of amplification primers (e.g., PCR primers) that correspond to the predetermined 5′ and 3′ ends of the nucleic acid fragment being assembled will preferentially amplify full length product in an exponential fashion. It should be appreciated that smaller assembled products may be amplified if they contain the predetermined 5′ and 3′ ends. However, such smaller-than-expected products containing the predetermined 5′ and 3′ ends should only be generated if an error occurred during assembly (e.g., resulting in the deletion or omission of one or more regions of the target nucleic acid) and may be removed by size fractionation of the amplified product. Accordingly, a preparation containing a relatively high amount of full length product may be obtained directly by amplifying the product of an assembly reaction using primers that correspond to the predetermined 5′ and 3′ ends. In some embodiments, additional purification (e.g., size selection) techniques may be used to obtain a more purified preparation of amplified full-length nucleic acid fragment.

When designing a plurality of oligonucleotides to assemble a predetermined nucleic acid fragment, the sequence of the predetermined fragment will be provided by the oligonucleotides as described herein. However, the oligonucleotides may contain additional sequence information that may be removed during assembly or may be provided to assist in subsequent manipulations of the assembled nucleic acid fragment. Examples of additional sequences include, but are not limited to, primer recognition sequences for amplification (e.g., PCR primer recognition sequences), restriction enzyme recognition sequences, recombination sequences, other binding or recognition sequences, labeled sequences, etc. In some embodiments, one or more of the 5′-most oligonucleotides, one or more of the 3′-most oligonucleotides, or any combination thereof, may contain one or more additional sequences. In some embodiments, the additional sequence information may be contained in two or more adjacent oligonucleotides on either strand of the predetermined nucleic acid sequence. Accordingly, an assembled nucleic acid fragment may contain additional sequences that may be used to connect the assembled fragment to one or more additional nucleic acid fragments (e.g., one or more other assembled fragments, fragments obtained from other sources, vectors, etc.) via ligation, recombination, polymerase-mediated assembly, etc. In some embodiments, purification may involve cloning one or more assembled nucleic acid fragments. The cloned product may be screened (e.g., sequenced, analyzed for an insert of the expected size, etc.).

In some embodiments, a nucleic acid fragment assembled from a plurality of oligonucleotides may be combined with one or more additional nucleic acid fragments using a polymerase-based and/or a ligase-based extension reaction similar to those described herein for oligonucleotide assembly. Accordingly, one or more overlapping nucleic acid fragments may be combined and assembled to produce a larger nucleic acid fragment as described herein. In certain embodiments, double-stranded overlapping oligonucleotide fragments may be combined. However, single-stranded fragments, or combinations of single-stranded and double-stranded fragments may be combined as described herein. A nucleic acid fragment assembled from a plurality of oligonucleotides may be of any length depending on the number and length of the oligonucleotides used in the assembly reaction. For example, a nucleic acid fragment (either single-stranded or double-stranded) assembled from a plurality of oligonucleotides may be between 50 and 1,000 nucleotides long (for example, about 70 nucleotides long, between 100 and 500 nucleotides long, between 200 and 400 nucleotides long, about 200 nucleotides long, about 300 nucleotides long, about 400 nucleotides long, etc.). One or more such nucleic acid fragments (e.g., with overlapping 3′ and/or 5′ ends) may be assembled to form a larger nucleic acid fragment (single-stranded or double-stranded) as described herein.

A full length product assembled from smaller nucleic acid fragments also may be isolated or purified as described herein (e.g., using a size selection, cloning, selective binding or other suitable purification procedure). In addition, any assembled nucleic acid fragment (e.g., full-length nucleic acid fragment) described herein may be amplified (prior to, as part of, or after, a purification procedure) using appropriate 5′ and 3′ amplification primers.

### Synthetic Oligonucleotides

It should be appreciated that the terms P Group and N Group oligonucleotides are used herein for clarity purposes only, and to illustrate several embodiments of multiplex oligonucleotide assembly. The Group P and Group N oligonucleotides described herein are interchangeable, and may be referred to as first and second groups of oligonucleotides corresponding to sequences on complementary strands of a target nucleic acid fragment.

Oligonucleotides may be synthesized using any suitable technique. For example, oligonucleotides may be synthesized on a column or other support (e.g., a chip). Examples of chip-based synthesis techniques include techniques used in synthesis devices or methods available from Combimatrix, Agilent, Affymetrix, or other sources. A synthetic oligonucleotide may be of any suitable size, for example between 10 and 1,000 nucleotides long (e.g., between 10 and 200, 200 and 500, 500 and 1,000 nucleotides long, or any combination thereof). An assembly reaction may include a plurality of oligonucleotides, each of which independently may be between 10 and 200 nucleotides in length (e.g., between 20 and 150, between 30 and 100, 30 to 90, 30-80, 30-70, 30-60, 35-55, 40-50, or any intermediate number of nucleotides). However, one or more shorter or longer oligonucleotides may be used in certain embodiments.

Oligonucleotides may be provided as single stranded synthetic products. However, in some embodiments, oligonucleotides may be provided as double-stranded preparations including an annealed complementary strand. Oligonucleotides may be molecules of DNA, RNA, PNA, or any combination thereof. A double-stranded oligonucleotide may be produced by amplifying a single-stranded synthetic oligonucleotide or other suitable template (e.g., a sequence in a nucleic acid preparation such as a nucleic acid vector or genomic nucleic acid). Accordingly, a plurality of oligonucleotides designed to have the sequence features described herein may be provided as a plurality of single-stranded oligonucleotides having those feature, or also may be provided along with complementary oligonucleotides.

In some embodiments, an oligonucleotide may be amplified using an appropriate primer pair with one primer corresponding to each end of the oligonucleotide (e.g., one that is complementary to the 3′ end of the oligonucleotide and one that is identical to the 5′ end of the oligonucleotide). In some embodiments, an oligonucleotide may be designed to contain a central assembly sequence (designed to be incorporated into the target nucleic acid) flanked by a 5′ amplification sequence (e.g., a 5′ universal sequence) and a 3′ amplification sequence (e.g., a 3′ universal sequence). Amplification primers (e.g., between about 10 and about 50 nucleotides long, between about 15 and about 45 nucleotides long, about 25 nucleotides long, etc.) corresponding to the flanking amplification sequences may be used to amplify the oligonucleotide (e.g., one primer may be complementary to the 3′ amplification sequence and one primer may have the same sequence as the 5′ amplification sequence). The amplification sequences then may be removed from the amplified oligonucleotide using any suitable technique to produce an oligonucleotide that contains only the assembly sequence.

In some embodiments, a plurality of different oligonucleotides (e.g., about 5, 10, 50, 100, or more) with different central assembly sequences may have identical 5′ amplification sequences and identical 3′ amplification sequences. These oligonucleotides can all be amplified in the same reaction using the same amplification primers.

A preparation of an oligonucleotide designed to have a certain sequence may include oligonucleotide molecules having the designed sequence in addition to oligonucleotide molecules that contain errors (e.g., that differ from the designed sequence at least at one position). A sequence error may include one or more nucleotide deletions, additions, substitutions (e.g., transversion or transition), inversions, duplications, or any combination of two or more thereof. Oligonucleotide errors may be generated during oligonucleotide synthesis. Different synthetic techniques may be prone to different error profiles and frequencies. In some embodiments, error rates may vary from 1/10 to 1/200 errors per base depending on the synthesis protocol that is used. However, in some embodiments lower error rates may be achieved. Also, the types of errors may depend on the synthetic techniques that are used. For example, in some embodiments chip-based oligonucleotide synthesis may result in relatively more deletions than column-based synthetic techniques.

In some embodiments, one or more oligonucleotide preparations may be processed to remove (or reduce the frequency of) error-containing oligonucleotides. In some embodiments, a hybridization technique may be used wherein an oligonucleotide preparation is hybridized under stringent conditions one or more times to an immobilized oligonucleotide preparation designed to have a complementary sequence. Oligonucleotides that do not bind may be removed in order to selectively or specifically remove oligonucleotides that contain errors that would destabilize hybridization under the conditions used. It should be appreciated that this processing may not remove all error-containing oligonucleotides since many have only one or two sequence errors and may still bind to the immobilized oligonucleotides with sufficient affinity for a fraction of them to remain bound through this selection processing procedure.

In some embodiments of the invention, a sliding clamp technique may be used for enriching error-free oligonucleotides after hybridization of oligonucleotides that are designed to be complementary, provided that the ends are “blocked” to inhibit dissociation of the clamped form of MutS from any heteroduplexes that are present.

In some embodiments, a nucleic acid binding protein or recombinase (e.g., RecA) may be included in one or more of the oligonucleotide processing steps to improve the selection of error free oligonucleotides. For example, by preferentially promoting the hybridization of oligonucleotides that are completely complementary with the immobilized oligonucleotides, the amount of error containing oligonucleotides that are bound may be reduced. As a result, this oligonucleotide processing procedure may remove more error-containing oligonucleotides and generate an oligonucleotide preparation that has a lower error frequency (e.g., with an error rate of less than about 1/50, less than about 1/100, less than about 1/200, less than about 1/300, less than about 1/400, less than about 1/500, less than about 1/1,000, or less than about 1/2,000 errors per base.

A plurality of oligonucleotides used in an assembly reaction may contain preparations of synthetic oligonucleotides, single-stranded oligonucleotides, double-stranded oligonucleotides, amplification products, oligonucleotides that are processed to remove (or reduce the frequency of) error-containing variants, etc., or any combination of two or more thereof.

In some aspects, synthetic oligonucleotides synthesized on an array (e.g., a chip) are not amplified prior to assembly. In some embodiments, a polymerase-based or ligase-based assembly using non-amplified oligonucleotides may be performed in a microfluidic device. Oligonucleotides synthesized on an array may be cleaved and added to any suitable assembly reaction without amplification. These oligonucleotides can be synthesized without a 5′ and/or 3′ amplification sequence (e.g., without one or more sequences that correspond to a universal primer sequence). Accordingly, these oligonucleotides can be used directly in an assembly reaction without removing one or more flanking amplification sequences. In some embodiments, about 3, 4, 5, 6, 7, 8, 9, 10, or more non-amplified oligonucleotides can be assembled (if they have appropriate overlapping regions as described herein) in a single reaction. The assembled nucleic acid then may be amplified using 5′ and 3′ primers. In some embodiments, the 5′ and 3′ primers correspond to target nucleic acid sequences at the 5′ and 3′ end of the assembled nucleic acid. However, in some embodiments, each of the 5′-most and 3′-most oligonucleotides that were used in the assembly reaction contain a flanking universal primer sequence that can be used to amplify the assembled nucleic acid.

In some aspects, a synthetic oligonucleotide may be amplified prior to use. Either strand of a double-stranded amplification product may be used as an assembly oligonucleotide and added to an assembly reaction as described herein. A synthetic oligonucleotide may be amplified using a pair of amplification primers (e.g., a first primer that hybridizes to the 3′ region of the oligonucleotide and a second primer that hybridizes to the 3′ region of the complement of the oligonucleotide). The oligonucleotide may be synthesized on a support such as a chip (e.g., using an ink-jet-based synthesis technology). In some embodiments, the oligonucleotide may be amplified while it is still attached to the support. In some embodiments, the oligonucleotide may be removed or cleaved from the support prior to amplification. The two strands of a double-stranded amplification product may be separated and isolated using any suitable technique. In some embodiments, the two strands may be differentially labeled (e.g., using one or more different molecular weight, affinity, fluorescent, electrostatic, magnetic, and/or other suitable tags). The different labels may be used to purify and/or isolate one or both strands. In some embodiments, biotin may be used as a purification tag. In some embodiments, the strand that is to be used for assembly may be directly purified (e.g., using an affinity or other suitable tag). In some embodiments, the complementary strand is removed (e.g., using an affinity or other suitable tag) and the remaining strand is used for assembly.

In some embodiments, a synthetic oligonucleotide may include a central assembly sequence flanked by 5′ and 3′ amplification sequences. The central assembly sequence is designed for incorporation into an assembled nucleic acid. The flanking sequences are designed for amplification and are not intended to be incorporated into the assembled nucleic acid. The flanking amplification sequences may be used as universal primer sequences to amplify a plurality of different assembly oligonucleotides that share the same amplification sequences but have different central assembly sequences. In some embodiments, the flanking sequences are removed after amplification to produce an oligonucleotide that contains only the assembly sequence.

In some embodiments, one of the two amplification primers may be biotinylated. The nucleic acid strand that incorporates this biotinylated primer during amplification can be affinity purified using streptavidin (e.g., bound to a bead, column, or other surface). In some embodiments, the amplification primers also may be designed to include certain sequence features that can be used to remove the primer regions after amplification in order to produce a single-stranded assembly oligonucleotide that includes the assembly sequence without the flanking amplification sequences.

In some embodiments, the non-biotinylated strand may be used for assembly. The assembly oligonucleotide may be purified by removing the biotinylated complementary strand. In some embodiments, the amplification sequences may be removed if the non-biotinylated primer includes a dU at its 3′ end, and if the amplification sequence recognized by (i.e., complementary to) the biotinylated primer includes at most three of the four nucleotides and the fourth nucleotide is present in the assembly sequence at (or adjacent to) the junction between the amplification sequence and the assembly sequence. After amplification, the double-stranded product is incubated with T4 DNA polymerase (or other polymerase having a suitable editing activity) in the presence of the fourth nucleotide (without any of the nucleotides that are present in the amplification sequence recognized by the biotinylated primer) under appropriate reaction conditions. Under these conditions, the 3′ nucleotides are progressively removed through to the nucleotide that is not present in the amplification sequence (referred to as the fourth nucleotide above). As a result, the amplification sequence that is recognized by the biotinylated primer is removed. The biotinylated strand is then removed. The remaining non-biotinylated strand is then treated with uracil-DNA glycosylase (UDG) to remove the non-biotinylated primer sequence. This technique generates a single-stranded assembly oligonucleotide without the flanking amplification sequences. It should be appreciated that this technique may be used to process a single amplified oligonucleotide preparation or a plurality of different amplified oligonucleotides in a single reaction if they share the same amplification sequence features described above.

In some embodiments, the biotinylated strand may be used for assembly. The assembly oligonucleotide may be obtained directly by isolating the biotinylated strand. In some embodiments, the amplification sequences may be removed if the biotinylated primer includes a dU at its 3′ end, and if the amplification sequence recognized by (i.e., complementary to) the non-biotinylated primer includes at most three of the four nucleotides and the fourth nucleotide is present in the assembly sequence at (or adjacent to) the junction between the amplification sequence and the assembly sequence. After amplification, the double-stranded product is incubated with T4 DNA polymerase (or other polymerase having a suitable editing activity) in the presence of the fourth nucleotide (without any of the nucleotides that are present in the amplification sequence recognized by the non-biotinylated primer) under appropriate reaction conditions. Under these conditions, the 3′ nucleotides are progressively removed through to the nucleotide that is not present in the amplification sequence (referred to as the fourth nucleotide above). As a result, the amplification sequence that is recognized by the non-biotinylated primer is removed. The biotinylated strand is then isolated (and the non-biotinylated strand is removed). The isolated biotinylated strand is then treated with UDG to remove the biotinylated primer sequence. This technique generates a single-stranded assembly oligonucleotide without the flanking amplification sequences. It should be appreciated that this technique may be used to process a single amplified oligonucleotide preparation or a plurality of different amplified oligonucleotides in a single reaction if they share the same amplification sequence features described above.

It should be appreciated that the biotinylated primer may be designed to anneal to either the synthetic oligonucleotide or to its complement for the amplification and purification reactions described above. Similarly, the non-biotinylated primer may be designed to anneal to either strand provided it anneals to the strand that is complementary to the strand recognized by the biotinylated primer.

In certain embodiments, it may be helpful to include one or more modified oligonucleotides in an assembly reaction. An oligonucleotide may be modified by incorporating a modified-base (e.g., a nucleotide analog) during synthesis, by modifying the oligonucleotide after synthesis, or any combination thereof. Examples of modifications include, but are not limited to, one or more of the following: universal bases such as nitroindoles, dP and dK, inosine, uracil; halogenated bases such as BrdU; fluorescent labeled bases; non-radioactive labels such as biotin (as a derivative of dT) and digoxigenin (DIG); 2,4-Dinitrophenyl (DNP); radioactive nucleotides; post-coupling modification such as dR-NH₂ (deoxyribose-NH₂); Acridine (6-chloro-2-methoxiacridine); and spacer phosphoramides which are used during synthesis to add a spacer ‘arm’ into the sequence, such as C3, C8 (octanediol), C9, C12, HEG (hexaethlene glycol) and C18.

It should be appreciated that one or more nucleic acid binding proteins or recombinases are preferably not included in a post-assembly fidelity optimization technique (e.g., a screening technique using a MutS or MutS homolog), because the optimization procedure involves removing error-containing nucleic acids via the production and removal of heteroduplexes. Accordingly, any nucleic acid binding proteins or recombinases (e.g., RecA) that were included in the assembly steps is preferably removed (e.g., by inactivation, column purification or other suitable technique) after assembly and prior to fidelity optimization.

### Assembly of Variant Libraries

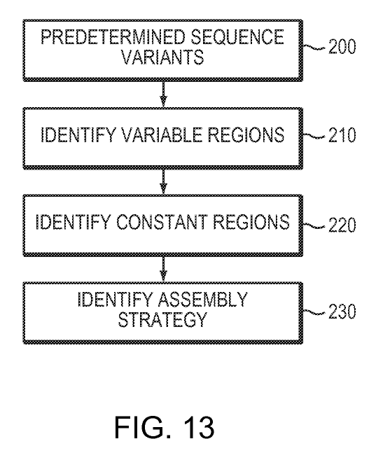

FIG. 13 illustrates an embodiment of an assembly strategy for a precise, non-random library (e.g., for a library that is predetermined, for example, by identifying or specifying a subset of all possible variants that are to be assembled). A non-random library may be assembled by combining two or more pools of predetermined nucleic acid variants (e.g., predetermined oligonucleotide variants), wherein each pool represents variants of a fragment of a reference sequence (e.g., of a starting sequence, for example a scaffold sequence or a natural sequence of which variants are being made). The resulting variants then may be assembled into longer fragments (e.g., intermediate fragments and/or a final full length library). In some embodiments, these steps are discrete, separate and sequential. In other embodiments, at least some of the reactions take place in a single reaction mixture.

FIG. 13 illustrates a non-limiting embodiment of such an assembly strategy of the invention. In act 200, predetermined sequence variants for a target nucleic acid are selected or obtained as described herein. Sequence variants may be variants of a single naturally-occurring protein encoding sequence. However, in some embodiments, sequence variants may be variants of a plurality of different protein-encoding sequences. In certain embodiments, the different protein-encoding sequences may be related (e.g., they code for similar or related proteins, proteins having similar or related functions, similar or related proteins from different species, or any combination thereof). In certain embodiments, library variants may be variants of a core scaffold sequence. The core scaffold sequence may be determined based on sequence comparisons (e.g., the scaffold sequence may be a consensus of sequences coding for similar or related proteins, proteins having similar or related functions, similar or related proteins from different species, or any combination thereof). In act 210, one or more variable regions are identified in a target nucleic acid. In some embodiments, a target nucleic acid is subdivided into a plurality of variable regions. In some embodiment, the entire length of the target nucleic acid is subdivided into consecutive variable regions. It should be appreciated that the length and number of variable regions selected may be related to the total number of variants to be made. For example, each variable region may be between about 10 and about 1,000 nucleotides long (e.g., about 50, about 100, about 200, about 500). However, shorter or longer variable regions may be selected. Each variable region may include between about 5 and about 10,000 different variants (e.g., about 10, about 50, about 100, about 1,000 or more). However, fewer or more variants may be included in a variable region. According to the invention, the theoretical final number of variants will be the product of the number of variants in each variable region that are combined together to form the final library. By assembling a plurality of relatively short variable regions each with relatively few variants, a relatively large number of final variants may be generated. Starting nucleic acids corresponding to each variant of a variable region may be independently synthesized (e.g., on separate columns, on surfaces such as chips, etc.) resulting in a precise synthesis of predetermined sequences (as opposed to a degenerate oligonucleotide that represents a plurality of predetermined sequences of interest in addition to a plurality of unwanted sequences). Accordingly, by combining precisely synthesized variable regions together, a high number of predetermined variants may be assembled precisely from a relatively low number of uniquely identified starting nucleic acids. In act 220, constant regions may be identified or selected. In some embodiments, no constant regions may be selected. However, in other embodiments one or more constant regions may be identified or selected (e.g., between variable regions). A constant region may be independently assembled and combined with one or more variable regions to produce a final library. Constant region(s) may be error-corrected, regardless of whether the variable region(s) are error-corrected. In some embodiments, each variable region is separated by a constant region. In some embodiments, each variable region has an invariant sequence at each end to be used for assembly with neighboring variable and/or constant regions. Accordingly, a variable region may be designed to include at least one invariant nucleotide at each end. In some embodiments, about 2, 3, 4, 5, 6, 7, 8, 9, 10, or more invariant nucleotides may be included at one or both ends of a variable region. The invariant nucleotides can be used (e.g., in combination with appropriate restriction enzymes such as Type IIS restriction enzymes) to generate complementary overhangs that can be used for ligating adjacent regions during assembly. In act 230, an assembly strategy is designed to determine the order in which the variable and constant regions are to be assembled and which regions and/or assembled fragments are to be error corrected.

Accordingly, a library may be designed and assembled to include all or substantially all of a large number of predetermined sequences of interest (e.g., at least about 100; at least about 1,000; at least about 10,000; at least about 100,000; at least about 10⁶; at least about 10⁷; at least about 10⁸; at least about 10⁹; at least about 10¹⁰ or more different nucleic acid variants). However, it should be appreciated that in some embodiments not all predetermined nucleic acids will be present in any given library. For example, between about 50% and about 100% (e.g., at least about 60%, at least about 65%, at least about 70%, at least about 75%, at least about 80%, at least about 85%, at least about 90%, at least about 95%, or at least about 99%) of predetermined sequences may be present. It also should be appreciated that a library assembled according to methods of the invention may include some errors that may result from sequence errors introduced during the synthesis of the assembly nucleic acids and/or from assembly errors during the assembly reaction. Error removal may be performed at one or more stages during assembly as described herein. In some embodiments, error removal may involve removing single base errors in the starting assembly nucleic acids or after one or more assembly stages (e.g., using a mismatch binding protein, sequencing, or other suitable techniques). In certain embodiments, error removal may involve size analysis or size selection of the starting assembly nucleic acids or after one or more assembly stages to remove assembled nucleic acids of unexpected sizes. However, unwanted nucleic acids may be present in some embodiments. For example, between 0% and 50% (e.g., less than about 45%, less than about 40%, less than about 35%, less than about 30%; less than about 25%, less than about 20%, less than about 15%, less than about 10%, less than about 5% or less than about 1%) of the sequences in a library may be unwanted sequences. Accordingly, different libraries with different types of variants (e.g., substitutions, deletions, insertions, etc., including silent mutations) or combinations thereof may be designed and/or assembled. Different libraries may have different levels of representativeness and/or density.

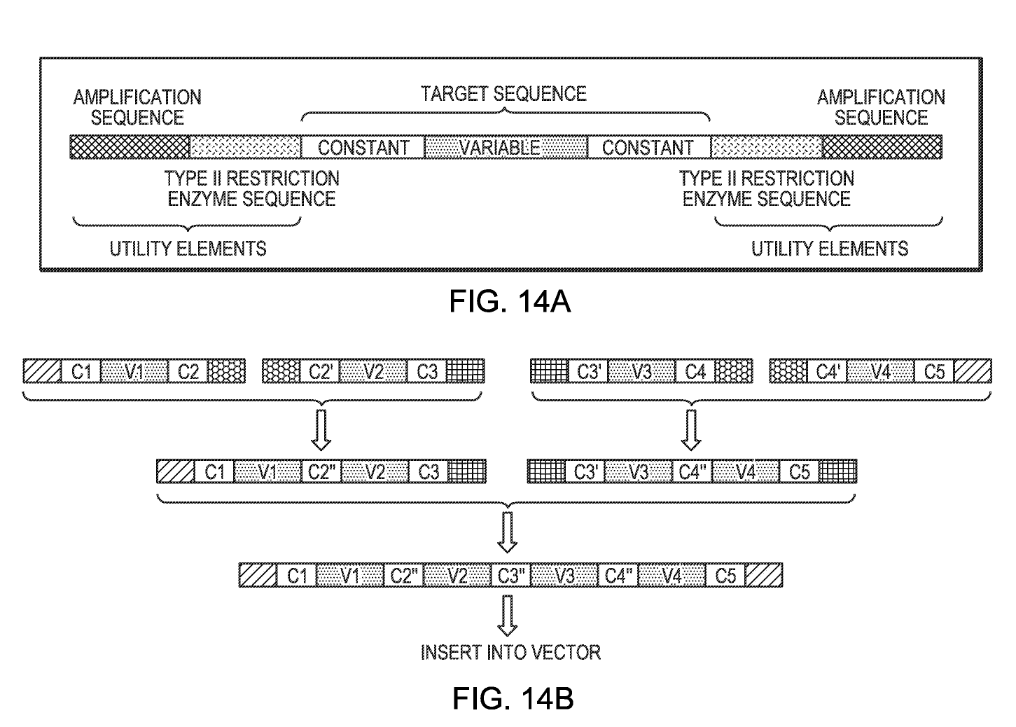

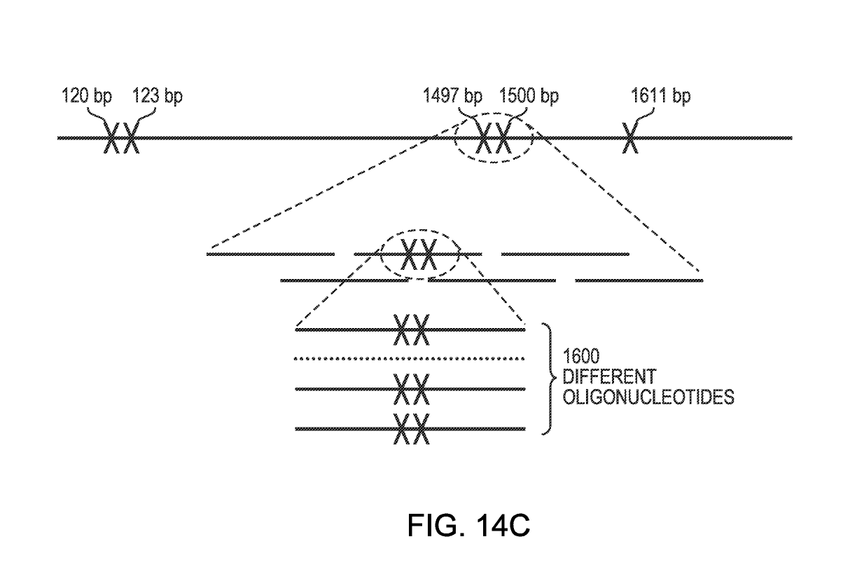

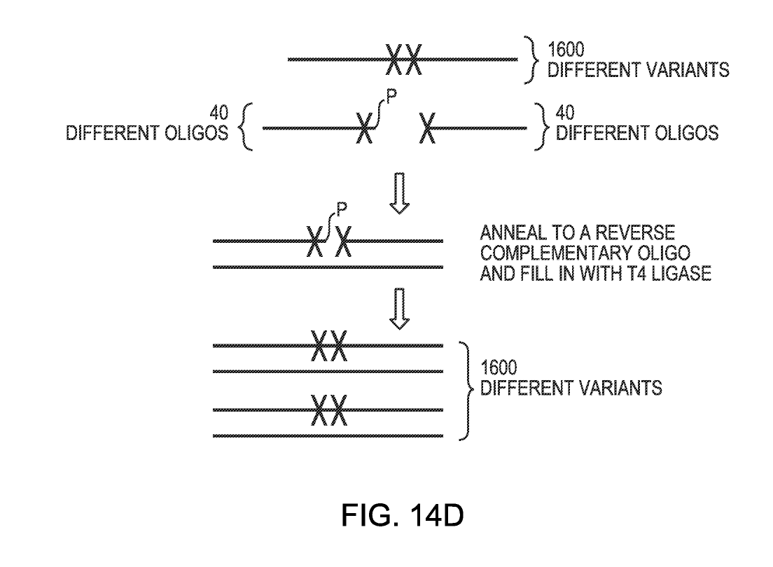

The invention further provides methods of designing nucleic acids (e.g., oligonucleotides) that are useful for constructing a library of desired (predetermined) variants. FIG. 14A schematically illustrates a design of an oligonucleotide useful for methods of the invention. It should be appreciated that each oligonucleotide fragment can be of any length, but is typically 40-200 bases long. In some embodiments, each oligonucleotide fragment includes two primary elements: target and utility elements. In some embodiments, a target element may include a variable region and a constant region on at least one end of the variable region. In some embodiments, a variable region is a segment of sequences that encode a peptide, within which one or more residues are selectively varied. In the diagram of FIG. 14A, a variable region is indicated in dark gray, flanked by constant regions shown in light gray. Additional sequences present on either end of the target sequence are collectively referred to as “utility elements”. The utility elements are designed to enable or facilitate various processes involved in the construction of a library, and may include sequences useful for selection, assembly and amplification and/or other processes. It is appreciated by one of ordinary skill in the art that the presence or the exact orientation or location of each of these utility elements may vary depending on the strategy of library construction as well as other factors, and it is not intended to be limiting. For example, in some embodiments, multiple amplification sequences may be present on one oligonucleotide. In some circumstances, an oligonucleotide is designed to include a universal amplification sequence. As used herein, the term “universal amplification sequence” means that a sequence used to amplify the oligonucleotide is common to a pool of mixed oligonucleotides such that all such oligonucleotides can be amplified using a single set of universal primers. In other circumstances, an oligonucleotide contains a unique amplification sequence. As used herein, the term “unique amplification sequence” refers to a set of primer recognition sequences that selectively amplifies a subset of oligonucleotides from a pool of oligonucleotides. In yet other circumstances, an oligonucleotide contains both universal and unique amplification sequences, which can optionally be used sequentially. In each case, amplification sequences may be designed so that once a desired set of oligonucleotides is amplified to a sufficient amount, it can then be cleaved by the use of an appropriate type IIS restriction enzyme that recognizes an internal type IIS restriction enzyme sequence of the oligonucleotide.

Utility elements of oligonucleotides may optionally include one or more spacer sequences. A “spacer sequence” is a sequence of any length, but typically 1-5 bases long, that can be inserted within the utility sequence to provide a means of adjusting the reading frame or the size (length) of the oligonucleotide itself. This is useful for, for example, size-based purification, or error removal. For example, a spacer sequence can be constructed between the amplification sequence and the type IIS restriction enzyme sequence. In some embodiments, where a subset of target variants includes a deletion or addition, resulting in a shortened or lengthened target sequence, the use of a spacer sequence may be desirable to compensate for the change in the total size (i.e., length). Size-based selection or purification of the oligonucleotides may be used.

FIG. 14A illustrates an embodiment of a configuration of oligonucleotides with utility sequences that include a pair of Type IIS restriction enzyme recognition sequences flanking an internal target sequence, and a pair of amplification sequence present on the 5′ end and the 3′ end of the oligonucleotides. The amplification sequences allow the use of complementary primers for amplifying the oligonucleotide containing the same amplification sequences. This is useful in a situation where a set of oligonucleotides are desired to be selectively amplified from a pool of mixed species of oligonucleotides. This is particularly useful when oligonucleotides are synthesized de novo using any chemical synthesis method such as on a surface (e.g., a microchip). Once so amplified, Type IIS restriction enzymes can be used to create a desirable overhang of the oligonucleotides so as to allow subsequent assembly of oligonucleotide fragments. Type IIS restriction enzymes cleave outside of their recognition site (typically 4-7 bp long). The distance between the recognition sequence and the proximal cut site varies from 1 to 20 bases, with a distance of 1 to 5 bases between staggered cuts, thus producing 1-5 bases single stranded cohesive ends, with 5′ or 3′ termini. Usually, the distance from the recognition site to the cut site is quite precise for a given type IIS enzyme. All exhibit at least partially asymmetric recognition. “Asymmetric” recognition means that 5′→3′ recognition sequences are different for each strand of the target DNA. To date, more than 80 type IIS restriction enzymes have been described.

In FIG. 14B, three generic type IIS restriction enzymes are depicted in an embodiment where they are used in a two-step construction of a library of variants derived from four fragments (e.g., pools) of oligonucleotides. The exact strategy for constructing a library may depend on a number of factors such as the complexity of target sequence and the number of variants to be included. Therefore, in some circumstances, construction may involve a single step, or two, three, four, five, or more steps.

FIG. 14B illustrates a non-limiting example of four oligonucleotide variant fragments to be assembled into a final product derived from four starting sequences. It should be noted that the number of fragments to be assembled (in this example, four) may be determined by multiple factors, such as the number of general areas that contain bases (residues) to be varied, and whether or not intervening constant regions exist between these variable regions, as well as the size of such segments. Each fragment represent a pool of variants containing one or more varied bases within the variable region and sequences that are common (identical) among the variants within the pool of fragments. For example, a variable region (e.g., VI) may encode a peptide that corresponds to a defined motif of a protein, where a set of residues are selected to be varied for altered function, stability and/or structure, etc. The adjacent constant regions represent sequences that are identical among the variants of the particular pool of oligonucleotides. Therefore, a constant region is at least one base, but preferably more (e.g., about 2, 3, 4, 5, 6, 7, 8, 9, 10, 10-100, 100-1,000, or more than 1,000). As will be clear to those skilled in the art, the number of fragments to be assembled into a final target sequence depends on multiple factors, such as the total length and complexity of the target. In some embodiments, a large number of relatively short fragments are assembled to generate target variants. In other embodiments, fewer fragments with relatively long or complex oligonucleotide are assembled to generate target variants. Yet other embodiments combine the two strategies to generate target variants.

Each of the four starting fragments contain a variable region, indicated as V1, V2, V3 and V4, respectively, as well as at least partially overlapping constant regions flanking the variable region. For the first fragment containing V1, constant regions shown as C1 and C2 flank the internal variable region, having the configuration: C1-V1-C2. The second fragment containing the variable region shown as V2 has the configuration C2′-V2-C3, where CT represents a partially overlapping sequence complementary to the C2 region of the first fragment. The two fragment variants also may contain a common type IIS restriction enzyme sequence, on the 3′ end of the first fragment and on the 5′ end of the second fragment. Accordingly, digestion of the two fragment variants with the appropriate type II restriction enzyme creates a complementary overhang on the fragments to be adjoined, yielding C″ as shown in FIG. 14B. Accordingly, using techniques well known in the art, the two fragments can be assembled to form C1-V1-C2″-V2-C3 as shown. Using a similar strategy, the other two fragments containing V3 and V4, respectively, are assembled in a separate reaction to form a second intermediate oligonucleotide, C3′-V3-C4″-V4-05 as shown in FIG. 14B. In some embodiments, such reactions may be combined, provided that the overhang termini on different fragments created by type IIS restriction enzyme digestions are sufficiently specific from one another. Therefore, when the constant regions (for example C2 and C4 in this example) are sufficiently diverse, these reactions may take place simultaneously. In contrast, when the constant regions share homology, separate reactions may be preferred. The two intermediate oligonucleotides are then assembled in a similar fashion to generate the target oligonucleotide, C1-V1-C2″-V2-C3″-V3-C4″-V4-05, as shown in the diagram. The remaining utility sequences on the 5′ terminus and 3′ terminus of the oligonucleotide may be used for inserting the product into a desired vector. The utility sequence may correspond to a type IIS restriction enzyme recognition sequence, or other restriction enzyme recognition sequence that is compatible to a vector of interest. In some embodiments, an adapter sequence corresponding to a type IIS restriction enzyme sequence present on the 5′- and 3′-ends of a target oligonucleotide is added to a vector as to render compatibility with the oligonucleotide to be inserted. It should be appreciated that this description is not limiting and a similar procedure may be used for fewer or more variable regions separated by constant regions. It also should be appreciated that each variable region described herein represents a plurality of variants (e.g., predetermined or specified variants) with than region. Accordingly, the assembly procedure described herein in the context of a variable region represents an assembly where a plurality of molecules having different sequence variants within the variable region are assembled (and wherein each variant molecule has the same constant region sequence within each different constant region described herein).

In some embodiments, variant positions in a target nucleic acid reside next to each other such that there is little intervening “constant” sequence between the two positions that are sought to be varied. In some embodiments, adjacent variant positions can be included in a variable region and different combinations of sequence variants can be individually synthesized for the variable region (e.g., within a region covered by a single oligonucleotide). However, in some embodiments, adjacent variant positions may be provided on separate nucleic acids (e.g., in separate nucleic acid pools) that are combined and assembled to provide further variation. According to aspects of the invention, adjacent variant positions on separate nucleic acids may be combined by ligation by using a complementary nucleic acid that overlaps at least the adjacent 5′ and 3′ regions. The complementary nucleic acid may be used to hybridize to the adjacent nucleic acids and provides a substrate for ligation. One or both of the adjacent nucleic acids may need to be phosphorylated (at the 3′ end or at the 5′ end) or otherwise modified to provide a substrate for a ligase enzyme. Any suitable ligase enzyme may be used (e.g., T4 ligase or any other suitable ligase). However, chemical ligation also may be used and one or both ends of the adjacent nucleic acids may need to be modified appropriately to provide a substrate for a chemical ligation reaction. According to aspects of the invention, the complementary nucleic acid should have sufficiently long 5′ and 3′ complementary regions (e.g., at least about 5, 5-10, at least about 10, 10-15, at least about 15, 15-20, at least about 20, 20-30, at least about 30, 30-50, or more nucleotides independently for each of the 5′ and 3′ complementary regions) so that sequence variants at the adjacent positions of interest do not differentially destabilize the hybridized ligation substrate. In some embodiments, the complementary nucleic acid may be complementary to most or all of the length of each of the adjacent nucleic acids (excluding non-complementary nucleotides at the one or few variant positions in the adjacent nucleic acids). It should be appreciated that if the 5′ and 3′ complementary regions are not sufficiently long, certain variants may hybridize less efficiently and therefore may be under-represented in an assembled library. In some embodiments, the complementary nucleic acid may be designed so that it is not complementary to any of the predetermined variants at the variant position, thereby to avoid preferential ligation of any of the different variants. Accordingly, the complementary nucleic acid may be designed to be complementary only to non-variant positions in at least the 3′ and 5′ regions of the adjacent nucleic acids to be assembled. However, in some embodiments, the complementary nucleic acid may be perfectly complementary to one of the variants. In some embodiments, the presence of one or two non-complementary nucleotides in some of the variants does not prevent them from being assembled into a library, particularly if the complementary regions are stabilized by a sufficient number of complementary non-variant positions. It should be appreciated that a complementary overlapping nucleic acid may be hybridized to two adjacent nucleic acids (e.g., oligonucleotides) and provide a substrate for ligation according to aspects of the invention even if the variable positions in the adjacent nucleic acids are not immediately adjacent but separated by one or more intervening constant positions.

FIG. 14C illustrates a non-limiting example where two variant positions are adjacent to each other along a sequence. Because of the configuration lacking a constant position between the two variant positions, a strategy such as that illustrated in the previous figure requiring constant nucleotides between variant positions is not applicable. In this nonlimiting example, assuming that there are 40 different variants at each of the two variable positions (adjacent variable codons) within an oligonucleotide, it would be necessary to generate 40×40=1,600 combinations of oligonucleotide variants using a conventional approach. To reduce the number of constructs necessary to generate all the combinations of variants, the instant invention discloses a faster, more economical approach of variant library construction, in which two variable sites are closely positioned along a sequence. According to the invention, a stretch of sequence containing two variable positions adjacent to each other is constructed as two short oligonucleotides separating the variable positions into two sets of oligonucleotides (see FIG. 14D). Accordingly, each of the short segments now contains a single variable position near one end of the segment. Again, assuming that there are 40 variants for each of the variable positions, these 40 oligonucleotides are synthesized for each of the segments. The end of the first segment is appropriately phosphorylated to promote the following reaction step (shown as P). A combination of the 40 variants from the first segment and the 40 variants from the second segment would yield all 1,600 possible combinations (40×40=1,600). To this end, a complement (a reverse complement) of the segment of nucleic acid construct that spans both of the short oligonucleotide segments is synthesized and annealed with pools of both of the short segments containing predetermined variant bases. Subsequently, the nick is filled in with a ligase (e.g., a T4 DNA ligase). It has been show that T4 ligase can catalyze this reaction even in the presence of mismatches at the end of the two segments (Cherepanov et al., J. Biochem. 129:61-68). As a result, all 1,600 combinations of oligonucleotides containing two adjacent variables may be generated. As used herein, T4 ligase refers to a DNA- or RNA-modifying enzyme that possesses the activity to fill in a nick in a double-stranded nucleic acid. T4 ligase catalyzes the formation of a phosphodiester bond between juxtaposed 5′ phosphate and 3′ hydroxyl termini in duplex DNA or RNA, using ATP as a cofactor. This enzyme will join blunt end and cohesive end termini as well as repair single stranded nicks in duplex DNA, RNA or DNA/RNA hybrids. T4 ligases are commercially available from, for example, New England Biolab (Beverley, Mass., U.S.A.). However, other suitable DNA or RNA ligases also may be used.

The library construction approach, as described herein, using T4 ligase-based nick filling in generating oligonucleotide variants, presents obvious advantage as compared to a conventional method discussed above in reducing the total number of oligonucleotides required. In the instant example, using this method, 81 (40+40+1=81) oligonucleotides 40 variants for each of the two segments plus a complementary oligonucleotide that spans the two segments—would suffice to generate the 1,600 combinations. In comparison, each of the 1,600 variants would have to be separately synthesized by a conventional method. Accordingly, when m and n are the number of variants at each position and there are two variable positions in a single oligonucleotide, the total number of variant oligonucleotides needed to make all combination is (m×n) using existing library construction strategies. If the length of nucleic acid to be assembled is 60 nucleotides, the total number of nucleotides required to be synthesized would be (m×n)×60. In contrast, using methods of the invention, only (m+n+1) oligonucleotides are required. Accordingly, the total number of nucleotides required to be synthesized is significantly less: (m+n)×30+(1×60). Aspects of the invention may be used to assemble variants where m and n independently represent different numbers of variants in adjacent regions of a nucleic acid being assembled. As discussed herein, the number of variants within a given region may represent variants at adjacent codons. Accordingly, each of N can be between 1 and 61 different amino acid encoding codons (and/or one or more of the three stop codons). It should be appreciated that this assembly technique may be used to prepare a subset of variants within a region that are then assembled with other variants to form a library of longer variant sequences. Accordingly, this assembly technique may be used to assemble pools of adjacent variants at two or more distinct locations within a construct that forms the basis of a library of sequence variants.

### Exemplary Applications of the Invention

Aspects of the invention may be useful for a range of applications involving the production and/or use of synthetic nucleic acid libraries. As described herein, the invention provides methods for producing synthetic nucleic acid libraries with increased fidelity and/or for reducing the cost and/or time of synthetic assembly reactions. The resulting assembled nucleic acids may be amplified in vitro (e.g., using PCR, LCR, or any suitable amplification technique), amplified in vivo (e.g., via cloning into a suitable vector), isolated and/or purified. An assembled nucleic acid library (alone or cloned into a vector) may be transformed into a host cell (e.g., a prokaryotic, eukaryotic, insect, mammalian, or other host cell). In some embodiments, the host cell may be used to propagate the nucleic acid. In certain embodiments, individual nucleic acids may be integrated into the genome of the host cell. In some embodiments, the nucleic acid may replace a corresponding nucleic acid region on the genome of the cell (e.g., via homologous recombination). Accordingly, nucleic acid libraries may be used to produce recombinant organisms. In some embodiments, a nucleic acid library may include entire genomes or large fragments of a genome that are used to replace all or part of the genome of a host organism. Recombinant organisms also may be used for a variety of research, industrial, agricultural, and/or medical applications.

Another aspect of the invention relates to construction of nucleic acid and polypeptide library for protein in vitro evolution. A combination of sequence and/or structure based, computational modeling; library construction; and medium-to-high-throughput protein expression and screening for a desired trait can be used to engineer polypeptide variants having substantially similar activity in the desired trait than a reference protein.

Computational protein modeling and design can be used for library design. Suitable computational algorithms include structure based and sequence based processing programs. In some embodiments, the reference protein has known three dimensional structure (e.g., there are three dimensional coordinates for each atom of the reference protein) which can be used to generate a scaffold protein. Generally this can be determined using X-ray crystallographic techniques, NMR techniques, de novo modeling, homology modeling, etc. Based on the three dimensional coordinates for each atom, optimal variants (e.g., having substantially similar coordinates and/or global energy) can be calculated. However, solving the structure of proteins is generally an expensive and time-consuming process. In examples where there is no known structure for a protein of interest, sequence based algorithms are preferred. Nucleic acid and/or amino acid sequence can be analyzed to determine segments of high level of conservation and/or functional importance. In some embodiments, these highly conserved segments (e.g., about 50%, 60%, 70%, 80%, 90%, 95% or more homology among different family members or species) can have a limited number of mutations. In some embodiments it can be undesirable to introduce mutations to conserved segments; instead, mutations can be preferably incorporated in less-conserved portions of the gene of protein. Accordingly, variants library having desired mutations can be designed. The mutations can be at predetermined sites. The mutations can be at random sites. The mutations can be substitution of amino acids by a desired subset of naturally and/or non-naturally occurring amino acids. The mutations can also be substitutions by any amino acids.

In one embodiment, a nucleic acid variant library designed from a reference gene can contain a predetermined number of mutations (n, n≧2). The predetermined number of mutations can be within one or more predetermined segments of the reference gene. The building blocks (e.g., oligonucleotides) can have 0, 1, 2, 3, 4, . . . , n mutations. The mutations within each oligonucleotide can be at the same or different position; and at any position, the nucleotide can be any one of A, T, G, and C. DNA synthesis and assembly technology can be performed according to any method or combination thereof described herein. Constructs or building blocks can be naturally-occurring (e.g., pieces of genomic DNA) or synthetic (e.g., through PCR or chemical synthesis). Further, synthetic constructs may be designed and/or engineered to have naturally-occurring properties (e.g., naturally occurring polynucleotide or polypeptide sequences) and/or non-naturally occurring characteristics (e.g., non-naturally occurring sequence variants, or non-natural combinations of functional elements).

Many of the techniques described herein can be used together, applying enrichment steps at one or more points to produce libraries containing long nucleic acid molecules having defined predetermined sequences. Correct sequence enrichment techniques of the invention can be applied to double-stranded nucleic acids of any size. For example, enrichment techniques using sliding clamp configurations of mismatch binding proteins may be used with oligonucleotide duplexes, nucleic acid fragments of less than about 100 to more than about 10,000 base pairs in length (e.g., 100mers to 500mers, 500mers to 1,000mers, 1,000mers to 5,000mers, 5,000mers to 10,000mers, etc.). In some embodiments, methods described herein may be used during the assembly of large nucleic acid molecules (for example, larger than about 5,000 nucleotides in length, e.g., longer than about 10,000, longer than about 25,000, longer than about 50,000, longer than about 75,000, longer than about 100,000 nucleotides, etc.). In an exemplary embodiment, methods described herein may be used during the assembly of an entire genome (or a large fragment thereof, e.g., about 10%, 20%, 30%, 40%, 50%, 60%, 70%, 80%, 90%, or more) of an organism (e.g., of a viral, bacterial, yeast, or other prokaryotic or eukaryotic organism), optionally incorporating specific modifications into the sequence at one or more desired locations.

Any of the nucleic acid products (e.g., including individual nucleic acids and nucleic acid libraries that are amplified, cloned, purified, isolated, etc.) may be packaged in any suitable format (e.g., in a stable buffer, lyophilized, etc.) for storage and/or shipping (e.g., for shipping to a distribution center or to a customer). Similarly, any of the host cells (e.g., cells transformed with a vector or having a modified genome) may be prepared in a suitable buffer for storage and or transport (e.g., for distribution to a customer). In some embodiments, cells may be frozen. However, other stable cell preparations also may be used.

Host cells may be grown and expanded in culture. Host cells may be used for expressing one or more RNAs or polypeptides of interest (e.g., therapeutic, industrial, agricultural, and/or medical proteins). The expressed polypeptides may be natural polypeptides or non-natural polypeptides. The polypeptides may be isolated or purified for subsequent use.

Accordingly, nucleic acid molecules generated using methods of the invention can be incorporated into a vector. The vector may be a cloning vector or an expression vector. In some embodiments, the vector may be a viral vector. A viral vector may comprise nucleic acid sequences capable of infecting target cells. Similarly, in some embodiments, a prokaryotic expression vector operably linked to an appropriate promoter system can be used to transform target cells. In other embodiments, a eukaryotic vector operably linked to an appropriate promoter system can be used to transfect target cells or tissues.

Transcription and/or translation of the constructs described herein may be carried out in vitro (i.e. using cell-free systems) or in vivo (i.e. expressed in cells). In some embodiments, cell lysates may be prepared. In certain embodiments, expressed RNAs or polypeptides may be isolated or purified. Nucleic acids of the invention also may be used to add detection and/or purification tags to expressed polypeptides or fragments thereof. Examples of polypeptide-based fusion/tag include, but are not limited to, hexa-histidine (His⁶) Myc and HA, and other polypeptides with utility, such as GFP, GST, MBP, chitin and the like. In some embodiments, polypeptides may comprise one or more unnatural amino acid residue(s).

The resulting expression library can contain about 10, 20, 50, 100, 200, 300, 400, 500, 1,000, 2,000, 3,000, 4,000, 5,000, 10,000, or more than about 10,000 polypeptide variants. The variants can be subjected to a variety of screening techniques to obtain desired variants that are functionally substantially equivalent to the reference protein. For example, the desired variant can have a characteristic related to function, utility, source (e.g., species, experimental system, etc.), cell-type specific and/or species-specific properties (e.g., expression, stability, toxicity, susceptibility to cell-type or species specific nucleases or proteases, etc.), interoperability with other parts or segments, nucleic acid sequence, amino acid sequence, codon usage, molecular weight, tertiary structure, quaternary structure, mRNA secondary structure, post-translational modifications, reactivity, modification sites, modes of detection, polarity, solubility properties such as hydrophobicity/hydrophilicity, membrane permeability, stability, bioavailability, safety, toxicity, isoelectric point, charge, thermostability, melting temperature, annealing temperature, catalytic activity, side groups, topology, kinetic complexity, immunogenicity, environmental hazards, and any combination of any of the foregoing, or other features.

In one example, the desired trait is a biological activity at an elevated temperature. For example, the reference protein can have a per unit enzymatic activity that reduces as temperature increases. Such enzymatic activity reduction can be due to low thermostability (e.g., resulting in irreversible changes in chemical composition and/or physical structure). It can be therefore desirable to enhance (or at least substantially retain) the thermostability by screening for variants having increased per unit enzymatic activity at an elevated temperature compared to the reference protein. In some examples, the variants can have substantially the same or reduced per unit enzymatic activity, but can acquire other desirable traits such as higher solubility, higher safety, lower toxicity, etc. Various screening techniques thus can be combined to identify variants having one or more desired traits.

In some embodiments, antibodies can be made against polypeptides or fragment(s) thereof encoded by one or more synthetic nucleic acids.

In certain embodiments, synthetic nucleic acids may be provided as libraries for screening in research and development (e.g., to identify potential therapeutic proteins or peptides, to identify potential protein targets for drug development, etc.)

In some embodiments, a synthetic nucleic acid may be used as a therapeutic (e.g., for gene therapy, or for gene regulation). For example, a synthetic nucleic acid may be administered to a patient in an amount sufficient to express a therapeutic amount of a protein. In other embodiments, a synthetic nucleic acid may be administered to a patient in an amount sufficient to regulate (e.g., down-regulate) the expression of a gene.

It should be appreciated that different acts or embodiments described herein may be performed independently and may be performed at different locations in the United States or outside the United States. For example, each of the acts of receiving an order for a target nucleic acid, analyzing a target nucleic acid sequence, designing one or more starting nucleic acids (e.g., oligonucleotides), synthesizing starting nucleic acid(s), purifying starting nucleic acid(s), assembling starting nucleic acid(s), isolating assembled nucleic acid(s), confirming the sequence of assembled nucleic acid(s), manipulating assembled nucleic acid(s) (e.g., amplifying, cloning, inserting into a host genome, etc.), and any other acts or any parts of these acts may be performed independently either at one location or at different sites within the United States or outside the United States. In some embodiments, an assembly procedure may involve a combination of acts that are performed at one site (in the United States or outside the United States) and acts that are performed at one or more remote sites (within the United States or outside the United States).

### Automated Applications

Aspects of the invention may include automating one or more acts described herein. For example, a sequence analysis may be automated in order to generate a synthesis strategy automatically. The synthesis strategy may include i) the design of the starting nucleic acids that are to be assembled into the target nucleic acid, ii) the choice of the assembly technique(s) to be used, iii) the number of rounds of assembly and error screening or sequencing steps to include, and/or decisions relating to subsequent processing of an assembled target nucleic acid. Similarly, one or more steps of an assembly reaction may be automated using one or more automated sample handling devices (e.g., one or more automated liquid or fluid handling devices). For example, the synthesis and optional selection of starting nucleic acids (e.g., oligonucleotides) may be automated using an nucleic acid synthesizer and automated procedures. Automated devices and procedures may be used to mix reaction reagents, including one or more of the following: starting nucleic acids, buffers, enzymes (e.g., one or more ligases and/or polymerases), nucleotides, nucleic acid binding proteins or recombinases, salts, and any other suitable agents such as stabilizing agents. Automated devices and procedures also may be used to control the reaction conditions. For example, an automated thermal cycler may be used to control reaction temperatures and any temperature cycles that may be used. Similarly, subsequent purification and analysis of assembled nucleic acid products may be automated. For example, fidelity optimization steps (e.g., a MutS error screening procedure) may be automated using appropriate sample processing devices and associated protocols. Sequencing also may be automated using a sequencing device and automated sequencing protocols. Additional steps (e.g., amplification, cloning, etc.) also may be automated using one or more appropriate devices and related protocols. It should be appreciated that one or more of the device or device components described herein may be combined in a system (e.g. a robotic system). Assembly reaction mixtures (e.g., liquid reaction samples) may be transferred from one component of the system to another using automated devices and procedures (e.g., robotic manipulation and/or transfer of samples and/or sample containers, including automated pipetting devices, etc.). The system and any components thereof may be controlled by a control system.

Accordingly, acts of the invention may be automated using, for example, a computer system (e.g., a computer controlled system). A computer system on which aspects of the invention can be implemented may include a computer for any type of processing (e.g., sequence analysis and/or automated device control as described herein). However, it should be appreciated that certain processing steps may be provided by one or more of the automated devices that are part of the assembly system. In some embodiments, a computer system may include two or more computers. For example, one computer may be coupled, via a network, to a second computer. One computer may perform sequence analysis. The second computer may control one or more of the automated synthesis and assembly devices in the system. In other aspects, additional computers may be included in the network to control one or more of the analysis or processing acts. Each computer may include a memory and processor. The computers can take any form, as the aspects of the present invention are not limited to being implemented on any particular computer platform. Similarly, the network can take any form, including a private network or a public network (e.g., the Internet). Display devices can be associated with one or more of the devices and computers. Alternatively, or in addition, a display device may be located at a remote site and connected for displaying the output of an analysis in accordance with the invention. Connections between the different components of the system may be via wire, wireless transmission, satellite transmission, any other suitable transmission, or any combination of two or more of the above.

In accordance with one embodiment of the present invention for use on a computer system it is contemplated that sequence information (e.g., a target sequence, a processed analysis of the target sequence, etc.) can be obtained and then sent over a public network, such as the Internet, to a remote location to be processed by computer to produce any of the various types of outputs discussed herein (e.g., in connection with oligonucleotide design). However, it should be appreciated that the aspects of the present invention described herein are not limited in that respect, and that numerous other configurations are possible. For example, all of the analysis and processing described herein can alternatively be implemented on a computer that is attached locally to a device, an assembly system, or one or more components of an assembly system. As a further alternative, as opposed to transmitting sequence information (e.g., a target sequence, a processed analysis of the target sequence, etc.) over a communication medium (e.g., the network), the information can be loaded onto a computer readable medium that can then be physically transported to another computer for processing in the manners described herein. In another embodiment, a combination of two or more transmission/delivery techniques may be used. It also should be appreciated that computer implementable programs for performing a sequence analysis or controlling one or more of the devices, systems, or system components described herein also may be transmitted via a network or loaded onto a computer readable medium as described herein. Accordingly, aspects of the invention may involve performing one or more steps within the United States and additional steps outside the United States. In some embodiments, sequence information (e.g., a customer order) may be received at one location (e.g., in one country) and sent to a remote location for processing (e.g., in the same country or in a different country (e.g., for sequence analysis to determine a synthesis strategy and/or design oligonucleotides). In certain embodiments, a portion of the sequence analysis may be performed at one site (e.g., in one country) and another portion at another site (e.g., in the same country or in another country). In some embodiments, different steps in the sequence analysis may be performed at multiple sites (e.g., all in one country or in several different countries). The results of a sequence analysis then may be sent to a further site for synthesis. However, in some embodiments, different synthesis and quality control steps may be performed at more than one site (e.g., within one county or in two or more countries). An assembled nucleic acid then may be shipped to a further site (e.g., either to a central shipping center or directly to a client).

Each of the different aspects, embodiments, or acts of the present invention described herein can be independently automated and implemented in any of numerous ways. For example, each aspect, embodiment, or act can be independently implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the present invention comprises at least one computer-readable medium (e.g., a computer memory, a floppy disk, a compact disk, a tape, etc.) encoded with a computer program (i.e., a plurality of instructions), which, when executed on a processor, performs one or more of the above-discussed functions of the present invention. The computer-readable medium can be transportable such that the program stored thereon can be loaded onto any computer system resource to implement one or more functions of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

It should be appreciated that in accordance with several embodiments of the present invention wherein processes are implemented in a computer readable medium, the computer implemented processes may, during the course of their execution, receive input manually (e.g., from a user).

Accordingly, overall system-level control of the assembly devices or components described herein may be performed by a system controller which may provide control signals to the associated nucleic acid synthesizers, liquid handling devices, thermal cyclers, sequencing devices, associated robotic components, as well as other suitable systems for performing the desired input/output or other control functions. Thus, the system controller along with any device controllers together form a controller that controls the operation of a nucleic acid assembly system. The controller may include a general purpose data processing system, which can be a general purpose computer, or network of general purpose computers, and other associated devices, including communications devices, modems, and/or other circuitry or components necessary to perform the desired input/output or other functions. The controller can also be implemented, at least in part, as a single special purpose integrated circuit (e.g., ASIC) or an array of ASICs, each having a main or central processor section for overall, system-level control, and separate sections dedicated to performing various different specific computations, functions and other processes under the control of the central processor section. The controller can also be implemented using a plurality of separate dedicated programmable integrated or other electronic circuits or devices, e.g., hard wired electronic or logic circuits such as discrete element circuits or programmable logic devices. The controller can also include any other components or devices, such as user input/output devices (monitors, displays, printers, a keyboard, a user pointing device, touch screen, or other user interface, etc.), data storage devices, drive motors, linkages, valve controllers, robotic devices, vacuum and other pumps, pressure sensors, detectors, power supplies, pulse sources, communication devices or other electronic circuitry or components, and so on. The controller also may control operation of other portions of a system, such as automated client order processing, quality control, packaging, shipping, billing, etc., to perform other suitable functions known in the art but not described in detail herein.

### Business Applications

Aspects of the invention may be useful to streamline nucleic acid library assembly reactions. Accordingly, aspects of the invention relate to marketing methods, compositions, kits, devices, and systems related to nucleic acid libraries using assembly techniques described herein.

Aspects of the invention may be useful for reducing the time and/or cost of production, commercialization, and/or development of synthetic nucleic acid libraries, and/or related compositions. Accordingly, aspects of the invention relate to business methods that involve collaboratively (e.g., with a partner) or independently marketing one or more methods, kits, compositions, devices, or systems for analyzing and/or assembling synthetic nucleic acid libraries as described herein. For example, certain embodiments of the invention may involve marketing a procedure and/or associated devices or systems involving nucleic acid libraries (e.g., libraries that encode filtered polypeptide sequences). In some embodiments, synthetic nucleic acids, libraries of synthetic nucleic acids, host cells containing synthetic nucleic acids, expressed polypeptides or proteins, etc., also may be marketed.

Marketing may involve providing information and/or samples relating to methods, kits, compositions, devices, and/or systems described herein. Potential customers or partners may be, for example, companies in the pharmaceutical, biotechnology and agricultural industries, as well as academic centers and government research organizations or institutes. Business applications also may involve generating revenue through sales and/or licenses of methods, kits, compositions, devices, and/or systems of the invention.

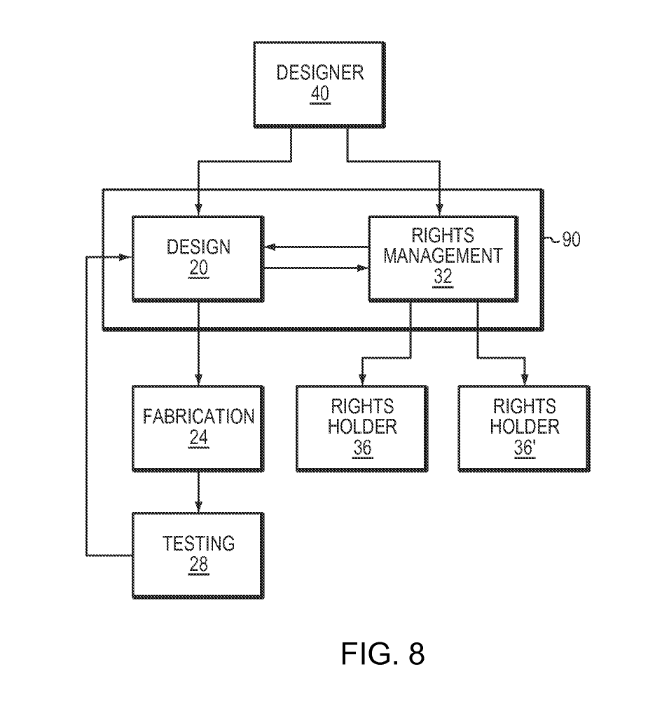

Other aspects of the invention relate to methods and systems for analyzing, designing, assembling, testing, and/or licensing molecular constructs that can be used in biological systems. Embodiments of the invention provide a system for designing, constructing and/or testing molecular constructs. FIG. 8 illustrates such a system which includes a design phase module 20, a fabrication phase module 24, a testing phase module 28, and a rights management module 32.

It should be appreciated that the term “construct” as used herein may include one or more structures along the entirety of the cascade of biological complexity, whether produced naturally or synthetically. Thus, for example, a construct may be an open reading frame or other DNA encoding or controlling expression of domains of a synthetic or naturally occurring protein, or plural DNAs which act cooperatively to achieve some goal, such as implementing a series of enzymatic changes in a substrate, defining a timing circuit in a cell, defining the parts of an expression vector, adapting a cell for use as a sensor of some xenobiotic in waste water, or implementing nanostructure designs. Alternatively the construct may be a protein having a specified set of properties, in which case the design may involve assembly at the DNA level (i.e., design of a precursor to the desired construct), expression and testing of a plurality of combinations of protein domains, and construction of various different candidate proteins by assembly of different genetic elements encoding the domains. In some embodiments, a construct may be an RNA molecule. In other embodiments, a bioconstruct may be a cell engineered to have some specific set of properties, or a collection of different cells that cooperate to achieve some function. In some embodiments, constructs may be molecular constructs comprising polynucleotide polymers. In other embodiments, constructs may be molecular constructs comprising polypeptide polymers. Accordingly, it should be appreciated that a construct may be divided into, or assembled from, smaller molecular segments (e.g., shorter poly- or oligo-nucleotides or shorter poly- or oligo-peptides) that may be referred to as building blocks in some embodiments of the invention. It also should be appreciated that a construct assembled using one or more methods or systems of the invention may be used as a building block for a larger construct or a biological system (e.g., a larger engineered polypeptide, a larger engineered nucleic acid, a recombinant vector, a recombinant cell, etc.).

In embodiments of the invention, molecular segment building blocks also may include one or more structures along the entirety of the cascade of biological complexity, whether produced naturally or synthetically. Accordingly, molecular segment building blocks may comprise one or more nucleobases, natural nucleotides, unnatural nucleotides, nucleotide analogs, modified nucleotides, codons, nucleic acids, oligonucleotides, polynucleotides, natural amino acids, unnatural amino acids, amino acid analogs, modified amino acids, peptides, polypeptides, chemical moieties, small molecules, vectors, plasmids, restriction sites, primers, hybridization sites, selection markers, detection markers, linkers, labels, ligands, antigens, antibodies or fragment thereof, or any combination thereof. The constructs may be assemblies of multiple genes incorporated into vectors, chromosomes, genomes, and cells.

Embodiments of the invention will be described, by way of example only and not intending to limit the scope of the invention, as applied to building a gene or a protein from smaller building blocks such as, for example, nucleotides, oligonucleotides, polynucleotides, a transcription unit (an open reading frame plus regulatory elements), amino acids, peptides, polypeptides, or any other suitable building blocks.

In some embodiments, the design phase module 20 includes information on building blocks and processes that may be used to create a molecular construct of interest. The design phase module 20 may produce a design specification of one or more constructs according to certain design requirements provided by a designer in any suitable manner. It should be appreciated that in some embodiments, a designer may choose to fabricate a single construct. However, a system of the invention may be used to design, assemble, test, and/or license a library of constructs. In some embodiments, a designer may specify or enter design information in the form of one or more sequences (e.g., nucleic acid and/or polypeptide sequences) to be analyzed, fabricated, and/or tested. The design module may analyze and/or decompose this sequence information to identify sequence segments that may be evaluated (e.g., screened) independently or in combination against the data repository. However, in some embodiments, the design module may evaluate (e.g., screen) the entire sequence directly against information in the data repository without involving an act of decomposing the sequence information or identify sequence segments. In certain embodiments, a designer may specify or enter one or more structural properties, functional properties, species specific properties, any other suitable properties, and/or any combinations thereof that are desired (e.g., 2, 3, 4, 5, about 5 to 10, about 10 to 20, about 20 to 50, about 50 to 100, or more different properties or combinations thereof). The design phase module 20 may include information on components and processes that may be used to create one or more molecular construct(s) of interest. The design phase module may identify one or more molecular segments that provide these different properties and design one or more different constructs that satisfy the design criteria. In some embodiments, a plurality (e.g., 1, 2, 3, 4, 5, about 5 to 10; about 10 to 100; about 100 to 1,000; about 1,000-10,000; about 10,000 to 100,000; or more) different constructs may be provided by the design phase module that all satisfy the design criteria. In some embodiments, a user may specify how many different constructs are wanted. In some embodiments, the different constructs may be related (e.g., have related nucleic acid sequences, amino acid sequences, structural properties, functional properties, or any combination of two or more thereof). It should be appreciated that the nature of the design criteria may impact the number of possible different constructs that satisfy the design criteria (e.g., depending on whether specific sequences are provided and/or whether specific or general structural and/or functional properties of interest are provided). If a plurality of different constructs satisfy the design criteria, all or a subset of them may be fabricated and/or tested to determine whether one or more of them is preferred based on any suitable criteria (e.g., assembly, function, expression levels, etc.). The fabrication phase module 24 may be a laboratory (e.g., molecular, chemical or any other suitable) which is capable of building the molecular construct according to the specification created by the design phase module 20. The testing phase module 28 may be a testing laboratory (e.g., molecular, chemical or any other suitable) which is capable of testing the molecular construct fabricated by the fabrication phase module 24 to determine if the construct meets the design requirements.

The rights management module 32 may comprise a data repository that includes information identifying use restrictions on a plurality of construct building blocks that a designer may include in a design for a construct or a product produced by a succession of steps involving the construct and/or construct building blocks. The use restrictions may be legal rights (e.g., intellectual property rights (IPR)), or any other rights restricting the use of the construct and/or its building blocks imposed by various rights holders or other agents. For example, the use restrictions may be patent restrictions, transfer restrictions, commercialization restrictions, safety restrictions, governmentally imposed restrictions, field of use restrictions, and any other restrictions. The use restrictions may (optionally) also provide a notification that a construct building block must be used in a facility providing some special conditions, that use of the construct building block in combination with some other class of construct building blocks may constitute patent infringement, or any other suitable notice that may be helpful to designer.

In one embodiment, the rights management module 32 may also manage the licensing of rights and payment of licensing fees to the rights holders 36 and 36′ by the designer 40. It should be appreciated that any other agent may act on behalf of the designer 40 to negotiate license use and payment of licensing fees with the rights holders 36 and 36′. The rights management module 32 may include an accounts payable module distributing remuneration to the holders of the intellectual property rights (not shown). It should be appreciated that the rights management module 32 need not manage the licensing of rights in all embodiments.

It should be appreciated that in some embodiments, a system of the invention may include a restriction management module that includes information identifying any features (e.g., structural and/or functional properties and/or any other feature set characteristics described herein) that may be used to restrict the constructs or construct building blocks that are selected for assembly or use. In some embodiments, a user may determine threshold levels of these features that may be used to restrict the selection of constructs and/or construct building blocks that are used and/or assembled. Any feature described herein may be used, alone or in combination with one or more other features, as a basis restrict the selection of one or more constructs or construct building blocks. In some embodiments, a user may determine which feature(s) are used and which threshold levels are used as a basis for a design restriction. Accordingly, a restriction management module may be based on features other than rights restrictions. However, a restriction management module also may include rights restrictions. It should be appreciated that one or more restrictions on the constructs and/or construct building blocks (e.g., molecular segments) may be imposed on a method or system of the invention to limit the number of different constructs that satisfy certain initial design criteria.

Once the data repository of the system is populated, the system holder in one embodiment may act as a broker and not only inform the users of available licenses and their terms, but also act as an intermediary to obtain the requisite licenses for the user. For example, in one embodiment discussed below, the data repository may also include a license to any molecular segment that is associated with the use restriction.

Embodiments of the invention may provide a capability to facilitate payments of intellectual property royalties for a designed construct. For example, the intellectual property royalties for a designed construct may be predicated on the number of cells utilizing the intellectual property protecting the construct. In one embodiment, enforcement of royalty payments may be accomplished, for example, by allowing the cell to undergo a finite number of cell divisions before the cell dies (e.g., by the insertion of a synthetic biology cell division counter coupled to a cell death mechanism) or by only using cells (e.g., auxotrophic cells) that require proprietary and exhaustible co-factors to live.

The designer 40 may employ the design phase module 20 to design one or more constructs in any suitable manner (e.g., by specifying the construct building blocks and processes required to build the construct). Once the design of the construct is finalized, the rights management module 32 may be used to determine which use restrictions, if any, on the construct and/or its building blocks are contained in the system. In some embodiments, if the use restrictions comprise rights available for licensing, the rights management module 32 may provide a license including the terms required for licensing the rights. The designer can access the license for review or execution. Such a license may be, for example, in a printable format that the designer can print out, sign, and submit to a licensor or intermediary, or the license may be a so-called “click through” license that is agreed to electronically. In one embodiment, the designer 40 has the option of accepting the terms of the license or redesigning the construct. If the designer 40 accepts the terms of the license, the designer 40 may need to make a payment to the rights management module 32 for distribution to one or more of the rights holders 36 and 36′. If the rights required to use (in one form or another) the designed construct are not available for license, the designer 40 may return to the design phase module 20 to design a new construct which may avoid using any building blocks that are unavailable for license, or are not official at desirable licensing terms.

Upon completion of the design phase, the design may be provided (e.g., by the designer or automatically) to the fabrication phase module 24 to fabricate the designed construct(s). The fabrication phase module 24 may be a molecular, chemical or any other suitable laboratory which is capable of fabricating the construct. It should be appreciated that the fabrication phase module 24 may use any suitable resources to fabricate the construct. For example, the fabrication module may employ one or more automated laboratories (e.g., robotic nucleotide or robotic amino-acid polymer manufacturing facilities) or any other suitable facility. In some embodiments, fabrication may involve any suitable combination of chemical synthesis, and/or in vivo, and/or in vitro assembly.

Once the one or more constructs have been fabricated, they may be tested by the testing phase module 28 to determine if they meet the requirements specified by the designer 40. The requirement can be specified and tested in any suitable manner. The testing phase module 28 may be a molecular, chemical or any other suitable testing laboratory which is capable of testing the construct(s). It should be appreciated that the testing phase module 28 may use any suitable resources to test the construct(s). If the testing phase module 28 determines that the construct(s) meets the requirements specified by the designer 40, the work of the designer 40 is completed. If, however, the construct(s) fails to meet the specified requirements, the designer 40 may return to the design phase module 20 to redesign the construct(s) and repeat the process until the construct(s) is designed and successfully tested.

Methods of the invention also include methods of identifying a sequence that meets a “distant” constraint in steps. For example, a rule may evaluate for a homology with a reference or parent sequence that is less than 80%. If the reference sequence is used as a starting place in the model, in a single round of design and assays, it may not be possible to test all possible predictions that would meet such a specification. Without limitation, this could be due to limitations in the model (too much change from the reference or parent structure) or due to assay throughput limitations. In a case like this an expert (or programmatic algorithm) can use a series of softer constrains and multiple rounds of designing and testing to approach the solution. In the above example the constraint can be softened in a first round, for example, to 90% homology, and sequences that satisfy the rule be designed and tested. Those that do are inputs into the next round of design, where the homology can be further constrained to 80%.

In some embodiments of the invention, one or more of the modules 20, 24, and 28 may be located on a server accessible over the Internet, thereby allowing the designer 40 to remotely access the system from any desired location. In some embodiments, the designer 40, or any other user or an operator of the system, may transmit information on the construct specification or any information to be transferred between modules to remote locations for further processing, fabrication or testing of the construct. The transfer of information may occur between the modules using any appropriate channels, e.g., computer-readable media encoded with the information, over a private or public (e.g., the Internet) network, or otherwise.

It should be appreciated that although one illustrative embodiment is described herein in which a designer uses each of the modules discussed above to design, fabricate and test a construct, it is contemplated that not all the modules need be in the same facility, and that various combinations of the modules may be in different locations. For example, it is contemplated that the design module and rights management module may be used together in one facility, and that the fabrication and testing may take place at locations owned and operated by others. This is merely one example of the various configurations that are possible. In addition, it is contemplated that not all of the modules described above, nor features of each, be employed in all embodiments of the present invention. For example, it is contemplated that the design module and the rights management module may be used together to facilitate a design, but decoupled from any system for performing fabrication and testing. In addition, and as discussed above, it should be appreciated that the aspects of the present invention described herein that relate to procuring a license to any protected subject matter need not be employed in all embodiments of the present invention, as the rights management module 32 can alternatively simply notify the designer of any relevant rights without acting as an intermediary to obtain a license thereunder.

In some embodiments of the invention, the design phase module 20 may include a data repository comprising a library of constructs, construct building blocks, and/or any combination of constructs and construct building blocks. The library may be built in any suitable manner, and, in one embodiment, may be populated by collecting information from different sources. For example, designer 40 may submit a construct, one or more construct building blocks, or a combination of construct building blocks to the library for use by others.

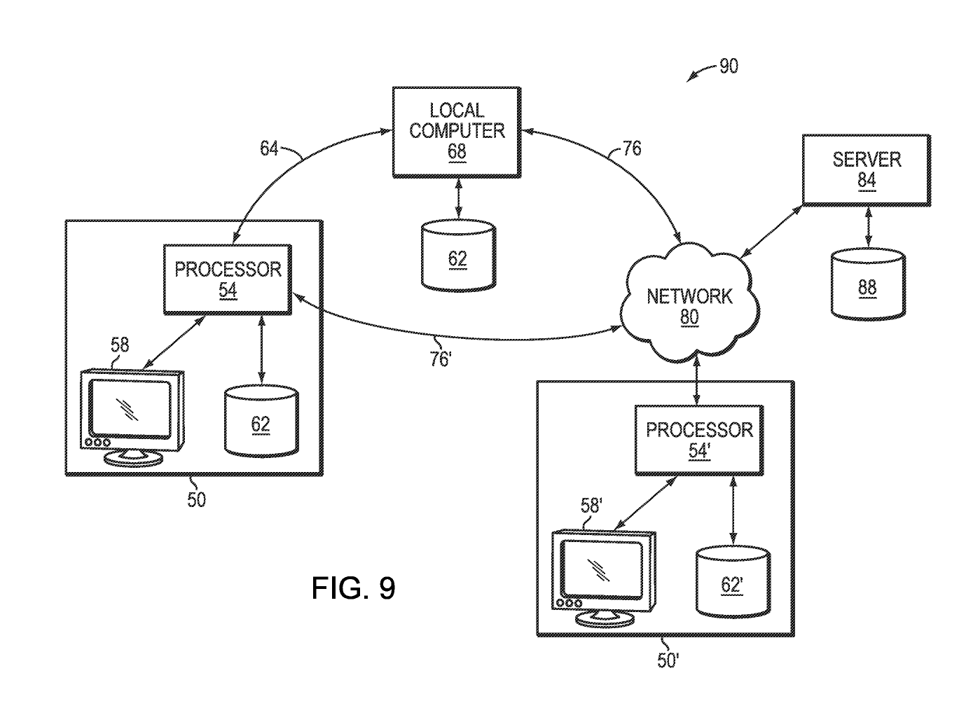

FIG. 9 illustrates an illustrative computing environment 90 on which embodiments of the invention may be implemented. It should be appreciated that the computing environment 90 is disclosed herein merely for illustrative purposes, and that the aspects of the present invention described herein can be implemented on any suitable computing environment, including a stand alone computer, or a distributed computing environment wherein multiple computers can distribute the functionality of the system described herein in any suitable manner and can communicate in any suitable manner (e.g., over a public or private network, or otherwise). The illustrative computing environment 90 includes a workstation 50 having a processor 54, a terminal 58, and a data storage device 62. In some embodiments, the workstation 50 may be a local stand-alone system (e.g., a desktop computer, laptop computer, or palmtop computer) which permits the user to utilize the functionality of the system. The terminal 58 may include any suitable input/output interfaces (e.g., a display, a mouse, a keyboard, a touch screen, a trackball, a digitizing table or any other suitable I/O device). The display may provide a graphical user interface for the system that, for example, enables the designer to specify at least a portion of a construct, receive feedback relating to use restrictions identified for the at least a portion of the construct, and exchange any of the other information described herein. The data storage device 62 may be any suitable storage device, including but not limited to, storage media such as ROMs, RAMs, floppy disks, CD-ROMs, DVDs, a high volume magnetic or optical disk drive, a distributed storage system implemented in a form of Redundant Arrays of Independent Disks (RAID) system, etc. In embodiments of the invention implemented on a stand-alone system, the same processor 54, terminal 58 and data storage device 62 are used for designing the construct as for managing the use restrictions, including intellectual property rights relating thereto.

In other embodiments, the system may not be implemented on a stand alone computer accessed by the designer. For example, the workstation 50 may be connected (e.g., by a local network 64 or otherwise) to a central local computer 68. Thus, the workstation 50 may act as the front-end to the local computer 68 so that the data storage device 62 on the workstation system 50 may be used to store only local data, e.g., the data input by the user. A data repository comprising the library of constructs, construct building blocks, any combination of constructs and construct building blocks and any of the other information described herein, may be stored at the central local computer 68 in storage device 72 that can be of any suitable type (e.g., a high volume magnetic or optical device or any of the other types described above for storage device 62). In these embodiments, the functions of the design phase module 20 and the rights management module 32 may be implemented by the local computer 68, using the workstation 50 as an input device. Alternatively, the design and rights management functions may be divided in any suitable way among the local computer 68 and the workstation 50.

In other embodiments of the invention, the local computer 68 and/or the workstation 50 may be connected, via connections 76 and 76′, respectively, through a wide area network 80, such as, for example, the Internet, to a server 84. The connections may be via any suitable communication media (e.g., wireless, wired, a combination thereof, etc.). A data storage device 88 (which may be any of the types described above for storage devices 62 and 72) that is coupled to the server 84 supplies data to multiple workstations 50, 50′ on the network 80. The workstation 50′ has a processor 54′ connected to data storage device 62′ and a terminal 58′. Because one or more servers 84 and data storage devices 88 may be processing simultaneously multiple requests from many clients (e.g., workstations 50, 50′) that may be located in different locations, the server 84 may be a high throughput device connected to a large volume high access rate data storage system 88, although the invention is not limited in this respect. The functions of the design phase module 20 and the rights management module 32 can be partitioned among the workstations 50, the local computer 68, and the server 84 in any suitable manner.

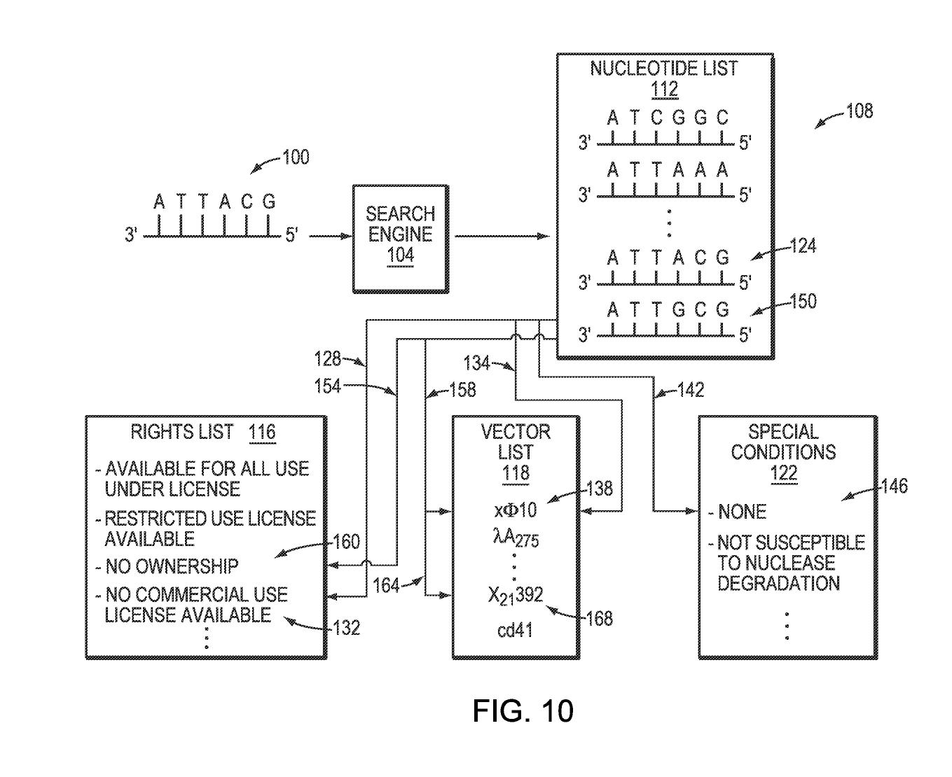

FIG. 10 is a schematic diagram illustrating an example of data structures used in the design phase and rights management phase modules according to some embodiments of the invention. When the designer 40 (e.g., a bioengineer or a scientist) desires to use a molecular segment 100, shown by way of example only as a nucleotide sequence 100, the designer 40 enters the sequence (e.g., using the workstation terminal 58). A database search engine 104 may be located on the processor 54, local computer 68, and/or server 84, depending upon whether the system is located at the workstation 50, local computer 68 or server 84 or distributed among them. The data storage device(s) 62, 72, and 88, associated with the search engine 104, may be queried to find a matching nucleotide sequence in the database 108, according to any suitable criteria. In some embodiments of the invention, the database 108 includes a list 112 of constructs and construct building blocks (e.g., nucleotide sequences shown by way of example in FIG. 10); a list of rights, or use restrictions, 116; and other suitable information, such as, for example, a list of transfection vectors 118 and a list of special information or conditions 122 relating to the molecular segments.

Various bioinformatics, machine learning, statistical learning, pattern recognition and other algorithms may be employed by the database according to embodiments of the invention. For example, the Smith-Waterman dynamic programming algorithm (T. Smith and M. Waterman. Identification of Common Molecular Subsequences. Journal of Molecular Biology, 147:195-197, 1981), heuristic algorithms such as BLAST (S. F. Altschul, W. Gish, W. Miller, E. W. Myers, and D. J. Lipman. Basic local alignment search tool. Journal of Molecular Biology, 215:403-410, 1990) and FASTA (W. R. Pearson. Rapid and sensitive sequence comparison with FASTP and FASTA. Methods in Enzymology, 183:63-98, 1985) may be used to compare a query nucleotide or protein sequence against the database of sequences, and uncover similarities and sequence matches. Furthermore, such machine learning algorithms as, for example, support vector machines (V. N. Vapnik. Statistical Learning Theory. Adaptive and learning systems for signal processing, communication, and control. Wiley, New York, 1998), Bayesian networks (J. Pearl. Probabilistic Reasoning in Intelligent Systems. Morgan Kaufmann, 1988), and Hidden Markov Models (L. R. Rabiner. A Tutorial on Hidden Markov Models and Selected Applications in Speech Recognition. Proceedings of the IEEE, 77:257-286, 1989), to name a few, and their more recent developments may be utilized to detect patterns within and among sequences (e.g., nucleotide and amino acids), classify the sequences, and make various predictions about the sequences. Any other suitable algorithms may be also employed.

In the example of FIG. 10, the database 108 is implemented as a linked list of records grouped according to a schema. However, it should be appreciated that the aspects of the present invention described herein are not limited to employing a database that is implemented in any specific manner, nor to even employing a database at all, as any suitably searchable data repository can be employed. In the example shown, the nucleotide segment 100 is found within the nucleotide list 112 in the database 108. In some embodiments of the invention, the found nucleotide sequence 124 (ATTACC) is forwardly and backwardly linked to the other lists 116, 118, and 122 contained in the database 108. The forward and backward linkages permit the user to search in any direction from any individual datum. Thus, the user (e.g., the designer or another) may specify a nucleotide sequence and obtain the rights corresponding to the sequence or specify a defined license and find all the building blocks to which the license applies. It should be appreciated that the specific implementation of the database 108 shown, wherein lists are linked forwardly and backwardly, is shown merely for illustrative purposes, as the data repository can be implemented in any suitable manner, as explained above. It should be appreciated that such forward and backward links may be used in connection with any construct (e.g., any nucleic acid and/or polypeptide construct).

The linkage 128 to the located sequence record 124 links the sequence record 124 to a specified rights record 132, which indicates that a commercial license for this sequence is not available. The linkage 128 also links, via a link 134, the sequence record 124 to a suggested vector record 138 (vector xΦ10) and, via a link 142, to a special conditions record 146 (no special conditions recorded). The fact that the nucleotide segment 100 is controlled by rights which permit no commercial use may make the nucleotide segment 100 unsuitable for its intended purpose. In such a case, in some embodiments, the search engine 104 can be instructed by the user to search for another nucleotide segment which may be a potential suitable replacement for the nucleotide sequence in question. The determination of a suitable replacement for any particular nucleotide sequence can be determined and searched for in any suitable manner, as the aspects of the present invention that relate to suggesting replacements are not limited to any particular technique for determining or locating suitable replacements. It should be appreciated that the identification of a replacement or substitute building block in connection with any methods or system described herein may be based on sequence information, structural information, functional information, or any combination thereof. For example, segments with similar sequences may be provided. In the case of a protein coding nucleic acid sequences, alternative sequences that encode that same or a related polypeptide sequence may be provided. In some embodiments, one or more related sequence motifs (e.g., from different organisms or species, related to a consensus sequence, etc.) may be provided. In the case of polypeptide sequences, alternative sequences having conserved amino acid substitutions may be provided. If building blocks are defined structurally, alternative building blocks having the same structure (e.g., the same secondary or tertiary structure). If building blocks are defined functionally, alternative building blocks having the same function may be provided. A building block may be defined based on any suitable function. For example, a function may be based on expression (e.g., transcription regulation, translation regulation, product stability, product function—enzymatic function, receptor binding, ligand binding—etc., or any combination thereof).

In one embodiment, the search engine 104 searches for an alternative nucleotide segment based upon the fact that the genetic code is degenerate. In this example, another nucleotide segment 150, which has a guanine residue at the third position from its 5′ end instead of the adenine, would also code for the amino acid proline and potentially is a replacement segment for the desired nucleotide segment 100. The forward and backward links 154 connect the record 150 in the nucleotide list 112 with a record 160 (no ownership) in the rights list 116, via links 158 and 164 to records 138 (xΦ10) and 168 (X₂₁392) of the vector list 118, respectively. In the example shown in FIG. 10, an additional link 172 to a record 146 (none) in the special conditions list 122 is shown. Thus, it appears that the found nucleotide segment 150 has similar characteristics to the nucleotide segment 100. That is, the same vectors may be used and no special handling conditions are required. The difference between the segments, in addition to the structural differences, is that one nucleotide sequence, 124, is owned and not licensable for commercial use, while another, the nucleotide sequence 150, is not owned and hence is available for use.

The results of the database search can be communicated to the user in any suitable manner. For example, the results may be returned to the terminal 58 for review by the user or printed as a report which, in turn, can be textual, graphical, or in any other suitable form. In addition, criteria can be specified such that the search engine 104 automatically searches for alternatives if any of the rights relating to the desired segments do not match the predetermined criteria.

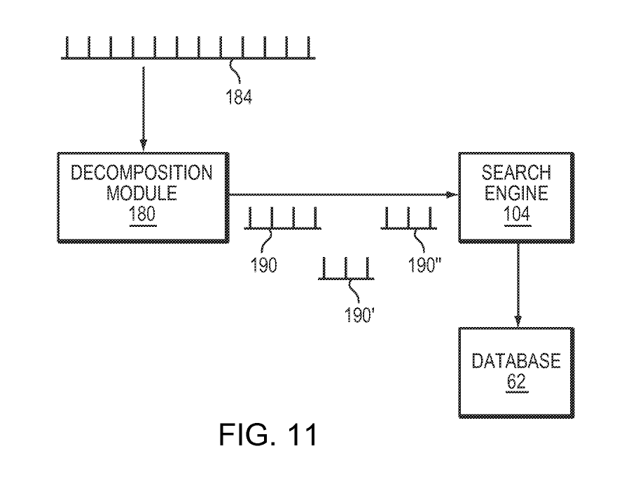

FIG. 11 illustrates an embodiment of the invention comprising a decomposition module 180 which permits the user to enter a desired final construct 184, which is shown, by way of example, as a polynucleotide (e.g., a gene), and the module fragments the construct 184 into a series of building blocks 190, 190′, and 190″. The segments 190, 190′, and 190″ may then be used as an input to the search engine 104 which searches the database 62 for the individual segments as described above in connection with FIG. 10. In some embodiments of the invention, one or more sequences (e.g., nucleic acid and or polypeptide sequences) may be decomposed into smaller sequence segments that are suitable for comparison with the information in the data repository. Accordingly, in some embodiments, the type of information in the data repository may impact the extent of decomposition performed by the decomposition module. The searches performed by a method or system of the invention may be based on sequence similarities with the sequence segments, predicted structural properties of the sequence segments, predicted functional properties of the sequence segments, or any combination thereof.

Although the description discusses exemplary embodiments relating to nucleotide segments, polynucleotides, legal rights, vectors and special conditions, other additional data lists such as promoters, enhancers, plasmids, selection markers, and others may be included or substituted.

Further, it should be understood that embodiments of the invention are not limited to use with nucleotides, but may also be used to design, construct, and test polypeptides, proteins, and molecular tags, to name a few. Accordingly, all examples of methods and/or systems described herein may be applied to any suitable construct (e.g., any nucleic acid or polypeptide construct) unless otherwise indicated.

It also should be appreciated that a construct of the invention may be a single linear polypeptide or polynucleotide polymer. However, in some embodiments, a construct may be a multimer of separate polymer subunits that interact or bind to each other (e.g., a dimer, trimer, tetramer, etc.). A multimer may be a homo-multimer or a hetero-multimer. Accordingly, in some embodiments of the invention, one or more restrictions described herein may specifically apply to rights and or characteristics of multimers and not the individual subunits of the multimers. For example, the user may wish to engineer an immunoglobin-G (IgG) antibody molecule. An IgG molecule is a quatramer constructed of two heavy polypeptide chains and two light polypeptide chains. The two heavy chains are bound together along their carboxyl portion by two disulfide bridges, forming a “Y” shaped structure. The two light chains are bound one to each arm of the “Y” of the heavy chain “Y” structure. The amino end of the quatramer, both of the heavy and light chains, forms the antigen binding site. The antigen binding site has both variable and hypervariable positions in which the amino acids, which make up the chains, vary significantly. It is this variability that permits the molecule to recognize and attach to specific antigens.

Thus, a user may wish to construct an IgG molecule having a specific amino acid sequence in the variable region of the molecule in order to direct the antibody to a specific antigen. The user may specify, for example, a light chain of the molecule, and then determine use restrictions and special conditions required to assemble the complete IgG molecule. Thus, any desired construct that may be assembled from smaller blocks may be checked for ownership and other restrictions that would prevent the construct from being used by its designer and/or made by an entity the designer is designing for. For example, constructs created by combinatorics, such as, for example, domain swapping, may be governed by intellectual property rights or other restrictions as described herein.

In some embodiments, various molecular segments within the database 108 may be linked to other molecular segments within the database 108 by commonality of features. The feature categories may include, for example, source, interoperability with other parts or segments blocks, tertiary structure, functionality, polarity, hydrophobicity, membrane permeability, FDA approval, bioreactivity, safety, toxicity, stability, bioavailability, environmental hazards, isoelectric point, charge, thermostability, melting temperature, annealing temperature, catalytic activity, side groups, topology, kinetic complexity, mRNA secondary structure, other suitable features, and any combination of any of the above. The linking according to common factors can be implemented in any suitable manner. In one embodiment, a series of tables may be defined, one for each feature category, where a unique identifier may be assigned to each of the molecular segments in the database 108. Each feature category table may list the unique identifier for each molecular segment belonging to the feature category. When a feature category is selected by the user, each unique identifier in the table acts as a pointer to the corresponding molecular segments in the database 108. Some of the feature categories may require one or more additional levels of indirection before arriving at the molecular segments list. For example, the category “functionality” may have additional subcategories, such as, for example, “receptor ligand”, “translational promoter”, “nuclease”, etc. If the subcategory “translational promoter” is selected, the table entries might point directly into the molecular segment list, while if the subcategory “nuclease” is selected, additional subcategories, such as “exonuclease” or “endonuclease”, may be required before their table entries point to the molecular segments in the database 108.

Thus, the user, through interaction with the database 108, independently, or via any other suitable means, may choose parts from which to build the construct, hypothesize how the parts will interact, and how the parts will operate in combination. The user inputs or retrieves an identifier piece or the whole building block that the user would like to use in the construct. Alternatively, the sequence of the entire construct may be entered, and the database is queried to identify what use restrictions are associated with the construct. The user thus avoids inadvertently using illegal molecular segments (e.g., DNA) and knows with certainty what rules apply with respect to the molecular segments being considered for inclusion into the construct.

Once the construct is designed, the designer or any other possessing the authority to act on behalf of the construct maker, may be presented with a license to use the components required by the design to make the construct. In some embodiments of the invention, the license may be a single sign-once license that obtains the proper license rights for the designer from all relevant rights holders 36 and 36′. In other embodiments, multiple licenses are generated and entered into (e.g., signed) by the designer 40 or other entity empowered acting on behalf of the construct maker. In this way, the designer 40 can simply pay once to a rights manager in the rights management module 32 for all the rights required to build the construct. In return, the rights manager of the rights management module 32 makes payments to the rights holders 36 and 36′, according to their licensing terms. It should be appreciated that multiple sign-once licenses may be required. For example, it is possible that a separate license may be required for an experimentation process, and a different license may be required for manufacturing. In various embodiments of the invention, it is contemplated that each type of license required a sign-once license.

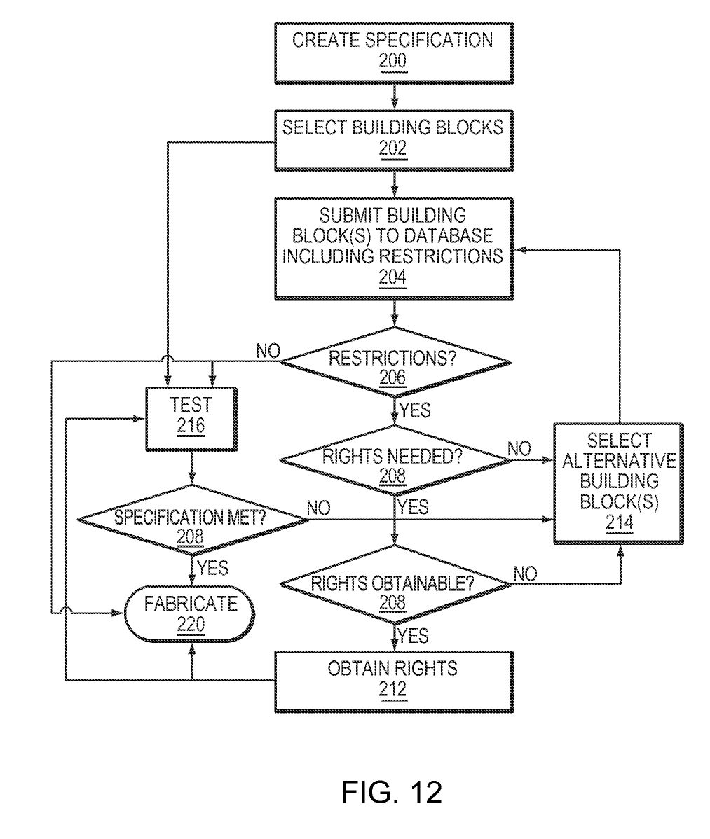

Some embodiments of the invention provide a method for designing, obtaining necessary rights, fabricating and testing the construct. FIG. 12 is a flowchart illustrating schematically one such method. In a step 200, a specification for a construct, construct building blocks, or any suitable combination thereof, is created. The specification may contain requirements for the desired construct and/or construct building blocks.

In a step 202, building blocks that may constitute the desired construct (e.g., polynucleotide or polyprotein) and/or construct building blocks may be selected. It should be appreciated that the building blocks may be selected in any suitable manner, e.g. specified by a designer (or any other user), selected automatically from a data repository or otherwise. It should also be appreciated that the desired construct and/or construct building blocks may be divided into any suitable (smaller) building blocks (e.g., molecular segments), depending on the specification and properties, structure and other features relating to the construct and/or construct building blocks. A decomposing module 180 described above in connection with FIG. 11 may be optionally employed to “decompose” the construct and/or construct building blocks into smaller building blocks. The data repository may be any suitable data storage (e.g., data storage devices 62, 72, and 88) comprising the library of constructs, construct building blocks, any combination of constructs and construct building blocks, use restrictions, and any other information, as discussed above.

The building blocks selected in step 202 may then be tested in a step 216. The test module 28 may be employed at the testing phase. Alternatively, in a step 204, the selected building blocks may be submitted to the data repository (e.g., data storage devices 62, 72, and 88, or other suitable data repositories) that includes, among other information, any suitable restrictions, including use restrictions and one or more features or feature sets related to building blocks. Each building block may be submitted separately, or any number of building blocks in any suitable combination may be submitted simultaneously. The building blocks selected in step 202 may also be submitted in any suitable form (e.g., as a specification, materials, or any other) directly for fabrication in a step 220. The fabrication module 24 or any other suitable facility may be employed.

A search engine may then identify, in step 206, whether any restrictions exist on the building blocks. If the answer is affirmative, in a step 208, it is determined whether any rights (e.g., legal rights) may be needed to use the building blocks, which may be done, for example, by querying the data repository discussed above. If rights are necessary, step 208 proceeds to a step 210, at which it is determined whether the rights are obtainable (e.g., a license may be obtained). If the answer is affirmative, the rights may be obtained in a step 212, which may be realized using the rights management module 32.

The “cleared” construct and/or construct building blocks may be fabricated in step 220. Optionally, the “cleared” construct and/or construct building blocks may be tested in step 216. It should be understood that use of a construct and/or construct building block may be determined to be hindered by both legal restrictions and restrictions related to certain functional, structural, or other features (e.g., a protein may cause toxic cell injury) related to the construct and/or construct building blocks. In this case, the design process may proceed towards selecting alternative block(s) in step 214 and, optionally, via step 210, towards obtaining rights in step 212.

If step 210 determines that the rights cannot be obtained, one or more alternative building blocks may be selected, in a step 214. It should be appreciated that the design, testing and fabrication modules may function interchangeably and that the described method may use these modules any suitable number of times and in any suitable order. In addition, other modules may be implemented as part of the system according to embodiments of the invention.

If it is determined in step 208 that no rights are needed, the process determines that existing restrictions identified in step 206 are related to some functional or structural properties or other features of one or more building blocks and proceeds to step 214 where one or more alternative building blocks may be selected. As discussed above, the step of searching for suitable alternative building blocks may be chosen to be performed automatically. If no restrictions were identified in step 206, the construct and/or construct building blocks may be tested in any desired way in step 216.

At any time during the testing phase or upon the completion of the testing phase, in a step 218, it may be determined whether the construct and/or construct building blocks meet the requirements specified in the specification created in step 200. If the requirements are determined to be met, the tested construct and/or construct building blocks may be fabricated, in step 220. The fabrication may also be conducted at an outside facility.

Thus, some embodiments of the invention provide capabilities to design and/or modify a design, obtain necessary rights, fabricate, and test a construct (e.g., a nucleic acid or other nucleotide polymer or a protein or other amino acid polymer). With all these capabilities provided by a single entity, the result is a one-stop facility that can be used to create incentives for designers to proceed from the acquisition of rights into an associated design/fabrication/testing facility by offering reduced fees and the ability to reduce the design-fabrication-testing cycle. It is possible for some constructs, such as a cell, to self-test after fabrication.

This method of conducting a construct designing business provides a business model which not only accrues to the benefits of the design/fabrication/testing facility but also to the designer by providing a system and method by which the designer can reduce the development time for each construct. The designer may also reduce this latency while making sure that the rights necessary to make the construct reside with the designer. It should be appreciated that although the example above described the rights library as containing the rights of third party rights holders, that the rights library alternatively may contain no proprietary construct components; proprietary components from only the owner of the design/fabrication/testing facility; collaborative third party rights; building blocks licensed from third parties and granted on a sublicense basis; and/or any other rights. The database may also comprise annotations in addition to rights and specifications associated with a building block, such as, for example, literature references, attributions, publications, patent references, purchasing information, and/or ordering capabilities.

Some embodiments of the invention enable the user to inform himself of often conflicting third party private or governmentally imposed legal use restrictions inuring to construct building blocks, and to select a functionally operative and legally permissible set of building blocks as candidates for inclusion in the design. This can be done prospectively during the design of the construct, by making inquiries about respective building blocks under consideration. This can also be done retrospectively upon completion of the design phase, by way of an audit or post design assessment of the designed construct and any of its building components. The system may also provide a mechanism through which standards of safety can be publicized and implemented, and third party patent rights and the like can be respected and enforced. Some embodiments of the invention may also provide a centralized, accessible source of data which enables users to make rational function-based decisions among design alternatives. Thus, embodiments of the invention provide a system that can be considered as a clearinghouse for clearing constructs and construct building blocks for use.

Embodiments of the invention may assist scientists, engineers and any other users engaged in the continuing elucidation of molecular biology mechanisms and in the creation and discovery of new and useful biological parts. Embodiments of the invention are directed to enabling diverse users to deposit voluntarily their discoveries and creations, or the sequence information defining them, with a data repository included in the system according to embodiments of the invention. The system may potentially act as a distributor to interested users. The users could specify the structure, sequence, use restrictions, royalty loads, compatibility data, functional data, and/or any other suitable information relating to created or discovered constructs or construct building blocks. As an example, an intellectual property control mechanism may be provided for a scientist who, for example, discovers and patents a new fluorescent protein that can be used as a marker of a successful DNA transfection. The scientist or any other agent authorized to act on his behalf may submit the sequence of the new biopart to the system according to embodiments of the invention, possibly also depositing samples, and providing descriptive data, use data, and/or specifications for the new protein. At the same time, use restrictions on the new protein may be submitted to the system repository by the designer of the protein or a corresponding authority (e.g., a university). The use restrictions might specify, for example, that the protein is freely available for academic or non-profit research, draws a $2.00 royalty per use for profit-based research, a royalty of 10% per unit if sold as a separate consumable reagent into the biological reagent market, and a royalty of 5% per unit incorporated in a kit or package off reagents and sold into the biological reagent market. Enforcement of royalty payments may be imposed in any suitable manner, examples of which are discussed above.

Once the construct is created, a therapeutic or diagnostic may be made utilizing the construct. The designer and the design/fabrication/testing facility owner and/or the rights owners can collaboratively market the therapeutic or diagnostic and divide the revenue thereby obtained.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device. Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or conventional programming or scripting tools, and also may be compiled as executable machine language code or intermediate code that is executed on a framework or virtual machine.

In this respect, the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1

### Nucleic Acid Fragment Assembly

Gene assembly via a 2-step PCR method: In step (1), a primerless assembly of oligonucleotides is performed and in step (2) an assembled nucleic acid fragment is amplified in a primer-based amplification.

A 993 base long promoter>EGFP construct was assembled from 50-mer abutting oligonucleotides using a 2-step PCR assembly.

Mixed oligonucleotide pools were prepared as follows: 36 overlapping 50-mer oligonucleotides and two 5′ terminal 59-mers were separated into 4 pools, each corresponding to overlapping 200-300 nucleotide segments of the final construct. The total oligonucleotide concentration in each pool was 5 μm.

A primerless PCR extension reaction was used to stitch (assemble) overlapping oligonucleotides in each pool. The PCR extension reaction mixture was as follows:

| | |
|---|---|
| oligonucleotide pool (5 μM total) | 1.0 μl (~25 nM final each) |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

Assembly was achieved by cycling this mixture through several rounds of denaturing, annealing, and extension reactions as follows:
start 2 min. 95° C.
30 cycles of 30 second at 95° C., 30 seconds at 65° C., 1 minute at 72° C.
final 2 minutes at 72° C. as extension step

The resulting product was exposed to amplification conditions to amplify the desired nucleic acid fragments (sub-segments of 200-300 nucleotides). The following PCR mix was used:

| | |
|---|---|
| primerless PCR product | 1.0 μl |
| primer 5′ (1.2 μM) | 5 μl (300 nM final) |
| primer 3′ (1.2 μM) | 5 μl (300 nM final) |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

The following PCR cycle conditions were used:
start 2 min. 95° C.
35 cycles of 30 second at 95° C., 30 seconds at 65° C., 1 minute at 72° C.
final 2 minutes at 72° C. as extension step

The amplified sub-segments were assembled using another round of primerless PCR as follows. A diluted amplification product was prepared for each sub-segment by diluting each amplified sub-segment PCR product 1:10 (4 μl mix+36 μl dH₂O). This diluted mix was used as follows:

| | |
|---|---|
| diluted sub-segment mix | 1.0 μl |
| dNTP (10 mM each) | 0.5 μl (250 μM final each) |
| Pfu buffer (10x) | 2.0 μl |
| Pfu polymerase (2.5 U/μl) | 0.5 μl |
| dH₂0 to 20 μl | |

The following PCR cycle conditions were used:
start 2 min. 95° C.
30 cycles of 30 second at 95° C., 30 seconds at 65° C., 1 minute at 72° C.
final 2 minutes at 72° C. as extension step

The full-length 993 nucleotide long promoter>EGFP was amplified in the following PCR mix:

| | |
|---|---|
| assembled sub-segments | 1.0 μl |
| primer 5′ (1.2 μM) | 5 μl (300 nM final) |
| primer 3′ (1.2 μM) | 5 μl (300 nM final) |
| dNTP (10 mM each) | 0.5 μl (250 μM final each) |
| Pfu buffer (10x) | 2.0 μl |
| Pfu polymerase (2.5 U/μl) | 0.5 μl |
| dH₂0 to 20 μl | |

The following PCR cycle conditions were used:
start 2 minutes at 95° C.
35 cycles of 30 second at 95° C., 30 seconds at 65° C., 1 minute at 72° C.
final 2 minutes at 72° C. as extension step

### Example 2

### Library Design for the Selection of Therapeutic Antibody Mimics

Certain embodiments of the invention may be exemplified by the design of a library for selecting therapeutic antibody mimics based on the tenth human fibronection type II domain (10Fn3), using pre-filtering for high solubility and low immunogenicity.

One possible library can be generated by randomizing twelve of the 94 amino-acid residues of 10Fn3, with the variability occurring in seven positions in loop BC (residues 23-29) and in five positions in loop DE (residues 52-56). The library will be made from two overlapping DNA fragments (“sub-libraries”), one encoding residues 1-47, and the other encoding residues 34-94. The library design and assembly may involve one or more of the following step.

1. An initial list of sequences will be generated for each sub-library by enumerating every possible permutation of the randomized positions. The resulting starting sub-libraries will contain 20⁷=10⁹ sequences (the N-terminal sub-library, “SL-N”) and 20⁵=10⁶ sequences (the C-terminal sub-library, “SL-C”).

2. A filtering step will be applied to each sub-library list that will remove all sequences that contain more than one tryptophan in the randomized region.

3. A filtering step will be applied to each sub-library list that will remove all sequences that contain one or more cysteines.

4. pI values will be calculated for each sequence on each list. All sequences with pI values between 6 and 9 will be removed from both lists.

5. Each sub-library list will be divided into two sublists. One list will contain the 1,000 sequences with the highest pI values (“SL-Nh” and “SL-Ch”); the other list will contain the 1,000 sequences with the lowest pI values (“SL-Nl” and “SL-Cl”).

6. The randomized region and the adjacent fixed positions for each of the 4,000 remaining sequences will be represented by a series of 9-mer, overlapping oligopeptides. Each of the peptides will be modeled into the peptide-binding site of all available MHC II structures. Each sequence that gave rise to an MHC-II-binding peptide will be removed from each list.

7. The remaining sequences on each list (SL-Nh, SL-Ch, SL-Nl, and SL-Cl) will be back-translated into DNA, optimized for codon usage and secondary-structure formation, and synthesized.

8. The physical DNA clones on each list (SL-Nh, SL-Ch, SL-Nl, and SL-Cl) will be combined to generate the four corresponding DNA pools, and will be PCR-amplified to 30 ug of DNA.

9. Pools will be combined pairwise: Pool H will result from combining pools SL-Nh and SL-Ch; pool L will result from combining pools SL-Nl and SL-Cl.

10. Pool H will be transformed into yeast strain EBY100 and recombined into a gapped plasmid used for yeast-surface display following standard protocol. Pool L will undergo the same procedure separately.

11. Transformed yeast cultures H and L will be grown separately and will have their complexity determined. Then the two cultures will be combined at same representation of each clone.

12. The resulting yeast library will be subjected to selection for binding to TNF-alpha using yeast-surface display, following standard protocols.

13. The selection is expected to yield a high proportion TNF-alpha-binding 10Fn3-like antibody mimics with high solubility and low immunogenicity.

### Example 3

### Engineering of Novel Thermostable DNA Polymerases

Companies are interested in novel versions of enzymes and target-binding proteins as well as novel versions of enzymes and target-binding proteins having improved function. For example, thermostable DNA polymerase I (tDps) with certain characteristics such as high processivity, high fidelity, longer polymerase chain (PCR) products, higher yields, and thermostability. It may be desirable for the engineered tDps to have a similar processivity and similar thermostability as that of thermostable DNA polymerase I from Thermus, but must have lower than 90% sequence identity to the thermostable DNA polymerase I from Thermus aquaticus (Taq).

More specifically, it may be desirable if one or more of the following parameters are met:
The processivity of the engineered tDp is no lower than that of Taq.
The specific activity is no lower than 90% of the specific activity of full-length Taq, which has been described as having a specific activity of 292,000 units/mg (Lawyer, et al., PCR Methods and Applications, 2:275-287 (1993)).
The half-life of the engineered tDp is at least nine minutes at 97.5° C., or the fraction of polymerase activity lost for the engineered tDp should be no more than the fraction of the enzymatic activity lost under the same conditions for Taq.
The engineered tDp has an increased fidelity compared to full-length Taq, or the engineered tDp has an error rate of 1 in 9,000 nucleotides or better when measured as described using published methods for measuring Taq fidelity (Tindall and Kunkel, Biochemistry, 27:6008-6013 (1988)), or an error rate that is no higher than 3 times that of the error rate of Pfu as described in Cline, et al. Nucleic Acids Res. 24:3546-3551 (1996).
The temperature optimum for polymerase activity for the engineered tDps is between about 75° C. and 80° C.

The buffer and other assay conditions used in measuring the above parameters are established, for example, based on the relevant literature and on experimental results, optionally provided by a collaborator, prior to measuring the above parameters.

A combination of structure-based, computational modeling; library construction; and medium-to-high-throughput protein expression and screening for enzymatic activity are used to engineer tDps with the required properties.

State-of-the-art in-vitro evolution of thermostable DNA polymerase I having improved function is performed. Protein engineering using computational protein modeling and design; library design; protein selection; molecular biology; protein expression, purification, and characterization; and polymerase assays are performed.

DNA synthesis and assembly technology are performed (see published applications referred to herein). The DNA synthesis and assembly technology allows fast and high-fidelity assembly of large numbers of unique genes, including defined-sequence libraries of up to 10¹³ unique, defined variants. In contrast to traditional libraries that rely on redundant nucleotides or codons (e.g., NNN, NNS, or NNK) to generate diversity in a library, libraries provided herein can be designed to contain only the variants pre-determined to be relevant to the project at hand (e.g., variants having the wild-type amino acid residue at one or more positions of motif A (amino acids 605-617 of the amino acid sequence for Taq polymerase I, Accession number P19821). For example, engineered variants tDps can have the wild-type Asp at position amino acid position 610 of P19821. As a consequence, libraries provided herein efficiently sample the sequence space most densely populated with solutions to the particular protein-engineering question, and avoid many inactive and unfolded sequences. The ability to make defined libraries valuable and even more valuable where information on protein structure is available. Availability of protein structure information can significantly accelerate the search for solutions.

I. Engineering of a Novel Thermostable DNA Polymerases Based on the Thermus aquiaticus DNA Polymerase I (Taq)

One or more characteristics of Taq polymerase is preserved while mutating more than 10% of its amino-acid residues, resulting in a novel tDp with less than 90% sequence identity to Taq polymerase of P19821. As large as possible a number of different residues is sampled or identified that can be mutated from the P19821 Taq polymerase sequence without diminishing, for example, its polymerase activity, and to identify active tDp variants with as low as possible a level of homology to the P19821 Taq polymerase sequence. The acceptable range of Kₘ, specific activity, processivity, fidelity, and/or other measurable properties of the engineered thermostable polymerase are defined relative to the properties of wild-type P19821 Taq polymerase sequence. The engineering of a novel tDps occurs in two Phases. In Phase I, libraries of novel tDps with lower than 90% sequence identity to the P19821 Taq polymerase sequence are designed computationally based on known Taq structures and sequences, then screened for activity. In Phase II, further libraries of novel tDps with even lower sequence identity to the P19821 Taq polymerase sequence are designed if the results of Phase I suggest that to be possible.

Two optional sequential parts of Phase I (Phase IA or Phase IB), and two optional versions of Phase II (Phase IIA or Phase IIB) are performed.

II. Phase IA: Simultaneous Design of tDp Variants with 88% and with 60-80% Sequence identity to the P19821 Taq Polymerase Sequence, and Testing of tDp Variants Having 88% Sequence Identity to the P19821 Taq Polymerase

A three-dimensional model of Taq polymerase is built based on the published crystal structure of DNA polymerase I from Thermus aquaticus P19821. Single-stranded DNA and/or dNTP will be modeled into the active-site, as identified in previously published studies. The extensive published information on enzymatic properties of Taq mutants is taken into account at this stage.

Next, up to 40 amino-acid residues per 100 amino-acid residues in length of Taq suitable for mutation are identified and ranked based on the details of the P19821 Taq polymerase model and on available sequence and structure-function information. The requirements satisfied by these residues can include one or more of the following criteria:
Solvent-accessibility of the side chain
Large distance from the active site
No involvement in interactions stabilizing secondary, tertiary, or quaternary structure
No reported mutations with a deleterious effect on Taq activity, stability, solubility, or expression level
Observed sequence variability at the position between aligned sequences of characterized thermostable DNA polymerases from different sources with sequence identity to the P19821 Taq polymerase of at least 30%

Mutations at the identified positions are designed with the aim to preserve Taq structure and function. Both structurally conservative mutations (e.g., serine to threonine) and mutations likely to increase solubility or stability of the enzyme (e.g., serine to glutamate) can be included.

Two libraries of novel tDp variants are designed using the mutations identified above:
The first library contains tDp variants with approximately 88% sequence identity to P19821 Taq polymerase, with the approximately 12 mutations per 100 amino acids in length of in each variant chosen from the most conservative positions and mutations identified above.
The second library contains tDp variants with sequence identity to P19821 Taq polymerase in the range of between 60 and 80%, i.e., with approximately 40 to about 20 mutations per 100 amino acids in length of the tDp variants. If fewer than 40 positions per 100 amino acids in length of Taq suitable for mutagenesis are identified at the modeling stage, the range of sequence identities to the P19821 Taq polymerase to be sampled or tested can be, for example, 65-80% or 70-85%.

DNA libraries encoding each library of variant tDps are constructed. Wild-type Taq polymerase is e also synthesized as a control. Codon usage is adjusted for high-level expression in E. coli. Codon usage is also optionally adjusted for high-level expression in one or more other host cells of choice.

In Phase IA, the DNA library encoding tDp variants with approximately 88% sequence identity to the P19821 Taq polymerase are transformed into E. coli, and 1,000 clones from the library are expressed, in parallel, on a small scale. E. coli lysates or partially purified extracts are screened for enzymatic activity in a published colorimetric assay. The assay can be approved or recommended by collaborator. Up to 100 variant clones with the highest level of the desired characteristic (such as polymerase activity or processivity) are sequenced, and up to five variant proteins with the highest level of the desired characteristic and with the most distinct sequences are expressed on a larger scale and purified to homogeneity. Enzymatic activity of the purified enzymes is characterized in detail. Genes for the variants that meet the project specifications are transferred to a collaborator.

II. Phase IB: Testing of tDp Variants with 60-80% Sequence Identity to the P19821 Taq Polymerase

In Phase IB, parallel enzyme expression and screen and larger-scale expression and characterization of hits proceeds as described in Phase IA, except that the library tested will be the library that contains the tDp variants with 60-80% sequence identity to the P19821 Taq polymerase.

III. Phase IIA: Engineering of tDp Variants with Low Sequence Identity to the P19821 Taq Polymerase Using Sequence-Verified Clones

Is it expected that the screen in Phase IA or Phase IB and the subsequent sequencing will yield a number of tDp variants with satisfactory enzymatic properties and with mutations in different positions. In Phase II, such mutations are combined to generate further variants with an even lower sequence identity to the P19821 Taq polymerase, but with a high probability of maintaining satisfactory enzymatic properties.

The details of the results from Phase I can determine the level of sequence identity between the P19821 Taq polymerase and the novel tDps constructed in Phase II. These details can also affect the number of novel tDp variants to be constructed to obtain active tDps at a lower homology level. For example, if numerous highly active tDp variants with diverse mutations are isolated at 88% sequence identity to the P19821 Taq polymerase, double the number of validated mutations are combined into a set of clones at 76% sequence identity to the P19821 Taq polymerase, and a smaller number of test clones are required.

Phase IIA describes the case where between 20-50 sequence-validated clones are constructed and tested to generate novel tDps at a lower level of sequence identity to the P19821 Taq polymerase. After screening for enzymatic activity, up to five variant proteins with the highest activity are expressed on a larger scale and purified to homogeneity, and their enzymatic activities are characterized in detail. Genes for the variants that meet the project specifications can be transferred to a collaborator.

IV. Phase IIB: Engineering of tDp Variants with Low Sequence Identity to the P19821 Taq Polymerase Using Libraries

Phase IIB describes the case where a library of 1,000 clones is constructed and tested to generate novel tDps at a lower level of sequence identity to the P19821 Taq polymerase. After screening for enzymatic activity, up to five variant proteins with the highest activity are expressed on a larger scale, purified to homogeneity, and their enzymatic activities characterized in detail. Genes for the variants that meet the project specifications can be transferred to a collaborator.

### Example 4

### Engineering of Novel Decarboxylases

Methods of the invention are further exemplified by the in silico design of a library of variants from a decarboxylase reference protein. Decarboxylases are carbon-carbon lyases that catalyze the hydrolysis of a carboxyl radical. In this example, the reference protein is subject to patent rights that also covered variants having decarboxylase activity and greater than a certain percentage amino acid sequence identity with the reference protein. A decarboxylase variant is desired that has substantially the same level of decarboxylase activity and thermostability as the reference protein, but an amino acid sequence identity that is less than that required to invoke the patent right associated with the reference protein.

As described below, a library is designed to include member variants having a specific percentage (X %) amino acid sequence identity. The specific percentage was selected to be below the percentage required to invoke the patent rights covering the reference protein. While a lower percentage of sequence identity may be acceptable for that purpose, variants having a higher percentages of sequence identity are more likely to exhibit at least equivalent function and stability as the reference protein. Thus, it is more efficient to focus the library on variants having the maximum acceptable sequence identity.

The first step in generating the library of variants of the reference protein is to identify amino acid residues and corresponding mutations that would likely result in minimal or no loss of activity or stability. In order to identify those residues, an in silico approach combining both structure-based and sequence-based methods is used.

In this example, the crystal structure for the decarboxylase reference protein is unknown. A structure for the reference protein is generated by homology modeling using crystal structure data from homologous decarboxylases. Briefly, the reference protein sequence is run through a sequence alignment program such as BlastP (NCBI) to identify homologous sequences. From the output, sequences for which the X-ray crystal structures are known are identified. Those sequences are aligned using the known structures in order to obtain the most accurate sequence alignment (i.e., a structure-based sequence alignment was produced). Next the sequence of the reference protein is aligned to the structure-based sequence alignment. Using that sequence alignment and a known decarboxylase structure as the template, a homology model was produced.

A variety of programs to facilitate such homology modeling are publicly available, for example, MODELLER, which is commercially available from Accelrys (at www.accelrys.com) or on the internet at www.alilab.org/modeller; see Sali and Blundell (1993) “Comparative protein modeling by satisfaction of spatial restraints” J. Mol. Biol. 234:779-815 and Marti-Renom et al. (2000) “Comparative protein structure modeling of genes and genomes” Annu Rev. Biophys. Biomol. Struct. 29:291-325. Other publicly available programs useful in homology modeling include PSI-BLAST (NCBI), THREADER (HGMP Resource Center, Hinxton, Cambs, CB10 1SA, UK), 3D-PSSM (three-dimensional position scoring matrix) (HGMP) and SAP programs.

The homology model for the reference protein is used in computational protein design methods to identify amino acid residues as candidates for mutagenesis. As described herein, a protein modeling program is used to calculate the predicted effect on of mutating each residue, in turn, to each of the other nineteen amino acids in a single point mutation scan. Alternatively, the predicted effect of multiple mutations can be calculated. In either case, the calculations provide a number of scores and a rankable total score for the resulting energies, thereby providing an indication of the predicted stability of the resulting variant.

In addition to the structure-based analysis, the sequence information for the reference protein is analyzed for candidate residues for mutation. Homologous amino acid sequences are identified through BLAST (NCBI) searching and aligned using pre-determined parameters and a threshold of identity. For example, that threshold identity may be 90%, 80%, 70%, 60%, 50%, 40%, 30% or less. Using computational protein design methods, each of the potential nineteen mutations for each residue is scored based on the conservative nature of the mutation, and the chemical similarity between the reference residue and the mutation.

The outputs of both the structure-based and the sequence-based methods are compiled and analyzed together in order to determine a set of candidate residues for mutation and preferred mutations at each such candidate residue. Residues or regions not believed to be important for activity are targeted such that mutations can be made without destroying activity. Further residues predicted to be on the surface of the reference protein are targeted. Alternatively, areas that may be important for biological activity or for structure may be targeted for conservative amino acid substitutions such that biological activity or polypeptide structure are not affected. In each case, chemically-similar or conservative amino acid substitutions are considered because they typically do not substantially change the structural characteristics of the reference sequence (e.g., a replacement amino acid should not tend to break a helix that occurs in the reference sequence, or disrupt other types of secondary structure that characterizes the reference sequence).

Once a set of candidate residues and corresponding mutations is identified, a library comprising variants having an amino acid sequence identity of X % with the reference protein can be generated. In order for each variant to have an amino acid sequence identity of X % with the reference protein, each variant must have a specific number (n) of amino acid mutations. All of the possible variant sequences can be generated in silico and, if desired, scored and ranked.

In order to produce the nucleic acids encoding the variant proteins, a nucleic acid library is constructed according to the DNA synthesis (e.g., PCR or chemical synthesis) and assembly (e.g., PCR or ligation) technology described herein. In this case, the nucleic acid encoding the reference protein is used as a template sequence for designing the multiple overlapping oligonucleotides that are used to assemble each of the nucleic acids encoding the variant proteins. For each oligonucleotide that encodes a candidate residue for mutation, a separate version of that oligonucleotide is synthesized for each mutation. In the event that the oligonucleotide encodes more than one candidate residue, only a defined number of mutated residues will be included in each version of the oligonucleotide. For example, if an oligonucleotide spans four candidate residues, each version of that oligonucleotide may be defined to include only two mutated residues, regardless of which two. By controlling the number of mutations included on each version of an oligonucleotide within any given set, the total number of mutations for each variant protein will remain constant regardless of which oligonucleotide is incorporated is into the final variant nucleic acid.

The library is introduced into suitable host cells and transformants are be selected based on selectable markers on the vector. Transcription and/or translation of the constructs described herein may be carried out in vitro (i.e. using cell-free systems) or in vivo (i.e. expressed in cells). Codon usage can be adjusted for high-level expression in the host cells. The reference decarboxylase is also synthesized as a control.

The resulting expression library containing protein variants are subjected to a variety of screening techniques to obtain desired variants that are functionally substantially equivalent to the reference decarboxylase—i.e., exhibiting equivalent decarboxylase activity and thermostability. Further libraries are produced for protein optimization purposes, such as increased per unit catalytic activity, increased thermostability, increased interoperability with other parts or segments, preferable codon usage, desirable post-translational modifications, useful modification sites, changed solubility, proper membrane permeability, increased stability, and/or increased biosafety, etc.

Once one or more desired variants are identified through the above phenotypic screening methods, the corresponding construct(s) can be retrieved and the nucleic acid sequence(s) encoding the desired variants can be determined (e.g., by sequencing). In particular, constructs that do not invoke patent rights can be identified. Thus through high-throughput in vitro evolution, novel decarboxylases with desired traits can be rapidly produced.

### EQUIVALENTS

The present invention provides among other things novel proteins and methods for designing and using the same. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

Reference is made to U.S. Published Application Nos. 2007/0037214, 2002/0045175, 2006/0160138, 2006/0281113, 2004/0019431, 2007/0004041, 2008/0064610, 2009/0136986 and PCT Publication Nos. WO08054543, WO08045380, WO08027558, WO07136840, WO07136835, WO07136834, WO07136833, WO07136736, WO07136736, WO07123742, WO07120624, WO07117396, WO07087347, WO07075438, WO07009082, WO07008951, WO07005053, WO06127423, WO06076679, WO06044956. All publications, patents and sequence database entries mentioned herein, including those items listed below, are hereby incorporated by reference in their entirety as if each individual publication or patent was specifically and individually indicated to be incorporated by reference. In case of conflict, the present application, including any definitions herein, will control.

What we claim is:

1. A method for determining a functional variant of a restricted protein, the method comprising:
identifying a restricted protein that exhibits a biological activity, said restricted protein being subject to a patent right;
determining at least one feature of said restricted protein, wherein said patent right is contingent upon said feature;
applying a computational design protocol to determine at least one portion of said restricted protein to which random mutations can be introduced, said protocol excluding any variant protein sequence that correspond to a variant protein having said feature;
generating a plurality of nucleic acid molecules encoding a plurality of variant proteins, wherein said plurality of variant proteins contain random mutations in at least one portion of said restricted protein;
expressing said nucleic acid molecules to produce said plurality of variant proteins; and
screening said plurality of variant proteins for said biological activity thereby to determine a functional variant of said restricted protein that is not subject to said patent right.

2. The method of claim 1, further comprising determining at least one structural characteristic of said restricted protein, said structural characteristic being correlated with said biological activity, and wherein said plurality of variant proteins comprise said structural characteristic.

3. The method of claim 1 wherein said patent right is a legal right for a rights-holder to exclude others from practicing a patented invention in the course of making, using, offering for sale, selling, or importing said restricted protein.

4. The method of claim 1 wherein said feature is an affirmative feature, and wherein said patent right is contingent upon the presence of said feature.

5. The method of claim 1 wherein said feature is a negative feature, and wherein said patent right is contingent upon the absence of said feature.

6. The method of claim 1 wherein said feature is a qualitative feature.

7. The method of claim 1 wherein said feature is an aspect of a nucleic acid or amino acid sequence corresponding to said restricted protein.

8. The method of claim 1 wherein said feature is an aspect of a tertiary structure of said restricted protein.

9. The method of claim 1 wherein said feature is a biological activity exhibited in an in vitro assay.

10. The method of claim 1 wherein said feature is a molecular weight of said restricted protein.

11. The method of claim 2 wherein said structural characteristic is qualitatively correlated with a level of biological activity exhibited by said restricted protein.

12. The method of claim 2 wherein said structural characteristic is an aspect of a nucleic acid or amino acid sequence corresponding to said restricted protein.

13. The method of claim 2 wherein said structural characteristic is an aspect of a tertiary structure of said restricted protein.

14. The method of claim 1 wherein said functional variant exhibits at least about 10%, 20%, 30%, 40%, 50%, 60%, 70%, 80%, 90%, 95%, 100%, 110%, 120%, 130%, 140% or 150% of the biological activity of said restricted protein.

15. The method of claim 1 wherein said plurality of variant protein sequences comprises at least about 1000, 2000. 3000, 4000, 5000, 6000, 7000, 8000, 9000, 10,000, 25,000, 50,000, 75,000, 100,000, 250,000, 500,000, 750,000, or 1,000,000 different sequences.

16. The method of claim 1 wherein said plurality of nucleic acid molecules comprises at least about 1000, 2000. 3000, 4000, 5000, 6000, 7000, 8000, 9000, 10,000, 25,000, 50,000, 75,000, 100,000, 250,000, 500,000, 750,000, or 1,000,000 different molecules having pre-defined sequences.

17. The method of claim 1 wherein at least about 50%, 60%, 70%, 80%, 90%, 95% or 99% of said plurality of nucleic acid molecules correspond exactly with said pre-determined sequences.

18. A method for designing a variant protein having a predetermined functional property, the method comprising:
providing an amino acid sequence of a reference protein having a predetermined functional property, wherein the reference protein has at least one associated feature;
determining if the at least one feature is subject to patent rights;
identifying at least one mutation tolerant amino acid position that does not affect the predetermined functional property;
modifying the feature by substituting at least one different amino acid at said mutation tolerant positions to generate a plurality of variants having alternate features that are not subject to the patent rights;
screening the plurality of variants in silico to produce a rank ordered list of variants;
generating nucleic acid molecules having predefined sequences that encode at least a subset of said plurality of variants;
expressing protein from the nucleic acid molecules to produce said variants; and
screening the variants for the predetermined functional property.

19. The method of claim 18 wherein the feature is selected from the group consisting of amino acid sequence, nucleic acid sequence, molecular weight, and tertiary structure.

20. A method for designing a variant protein having a predetermined biological activity, comprising:
(a) providing a sequence of a reference protein having the predetermined biological activity;
(b) identifying a plurality of mutation tolerant positions in a reference protein having a known biological activity by comparing its sequence or structure with of a plurality of related proteins having the same biological activity;
(c) screening a plurality a possible variants in silico to produce a rank ordered list of variants; and
(d) substituting the amino acids present at the highest ranked mutation tolerant positions to produce a first library of proteins variants having an amino acid sequence that is different to the reference protein.
(e) generating nucleic acid molecules that encodes at least a subset of said protein variants;
(f) expressing the nucleic acid molecules to produce said protein variants;
(g) screening the first library of variant proteins for said predetermined functional property; and
(h) selecting a first set of variant proteins having the least homology to the reference protein and the highest predetermined biological activity.

21. The method of claim 20, further comprising:
(i) screening the first set of variant proteins in silico to produce a rank ordered list of variants;
(j) substituting the amino acids present at the highest ranked mutation tolerant positions to produce a second library of proteins having an amino acid sequence that is different to the reference protein and to the first library of protein variants;
(k) generating nucleic acid molecules that encodes at least a subset of the protein variants from the second library;
(l) expressing the nucleic acid molecules from the second library to produce said protein variants from the second library;
(m) screening the protein variants from the second library for said predetermined functional property; and
(n) selecting a second set of variant proteins having the least homology to the reference protein and the highest predetermined biological activity.

22. A method of claim 21, further comprising repeating step (a) through (f) to select a third set of variant proteins having the least homology to the reference protein and the highest predetermined biological activity.

23. The method of claim 20, 21 or 22 wherein the variant protein has less than 95% homology to the reference protein sequence.

24. The method of claim 20, 21 or 22 wherein the variant protein has less than 90% homology to the reference protein sequence.

25. The method of claim 20, 21 or 22 wherein the variant protein has less than 80% homology to the reference protein sequence.

26. The method of claim 20, 21 or 22 wherein the variant protein has less than 70% homology to the reference protein sequence.

27. The method of any one of claims 20-26 wherein the variant protein has less than about 60% homology to the reference protein sequence.

28. The method of claim 20 wherein the least homology is no less than about 90%.

29. The method of claim 20 wherein the least homology is less than about 80%.

30. The method of claim 20 wherein the least homology is less than about 70%.

31. The method of any one of claims 20-26 wherein the variant protein has at least about 95% of the reference protein functional property.

32. The method of any one of claims 20-26 wherein the variant protein has at least about 90% of the reference protein functional property.

33. The method of any one of claims 20-26 wherein the variant protein has at least about 80% of the reference protein functional property.

34. The method of claim 20, wherein said comparing including aligning said reference protein and related protein amino acid sequences to make a sequence alignment.

35. The method of claim 28, wherein said method comprises comparing the amino acid sequence of a variable region of said reference protein to the variable region of said related proteins and substituting the amino acid in said variable region.

36. The method of any one of claims 20-26 wherein the reference protein and the related proteins have at least about 30% sequence identity.

37. The method of any one of claims 20-26 wherein the variant proteins and the reference proteins have substantially equivalent structural properties.

38. The method of claim 37 wherein the structural property is thermostability, solubility, expression level or any combination thereof.

39. The method of any one of claims 20-26 wherein substitution in a mutation tolerant position does not reduce the protein functional property, biological activity, stability, solubility, and expression level.

40. The method of any one of claims 20-26 wherein the mutation tolerant position comprises solvent-accessible amino acids, amino-acids at least a pre-determined distance from the active site, amino acids not involved in stabilizing secondary, tertiary or quaternary protein structure, or any combination thereof.

41. A variant protein designed by any one of the methods of claims 20-26.

42. A nucleic acid encoding a protein designed by any one of the methods of claims 20-26.

43. A method of designing a library of variant proteins, the method comprising:
identifying a reference protein that exhibits a biological activity;
determining at least one qualitative feature of said reference protein, said qualitative feature being divisible into at least a first and a further constrained second gradient level;
applying to said reference protein a design algorithm to generate a plurality of variant protein sequences that comprise said qualitative feature corresponding to said first gradient level;
generating a plurality of nucleic acid molecules having predefined sequences encoding said plurality of variant proteins;
expressing said nucleic acid molecules to produce said variant proteins;
screening said variant proteins for biological activity to identify a functional variant protein exhibiting said biological activity;
repeating said applying, generating and expressing steps with said functional variant protein as the reference protein and using a design algorithm to generate a second plurality of variant protein sequences that comprise said qualitative feature corresponding to said second gradient level; and
screening said second plurality of variant protein sequences to identify a functional variant protein exhibit said biological activity and have said qualitative feature corresponding to said second gradient level.

44. The method of claim 44, further comprising repeating said applying, generating expressing and screening steps with further constrained levels of said qualitative feature until a functional variant protein with target level of said qualitative feature is determined.

45. A method for determining a variant of a restricted nucleic acid, the method comprising:
identifying a restricted nucleic acid having a desired property, said restricted nucleic acid being subject to a patent right;
determining at least one feature of said restricted nucleic acid, wherein said patent right is contingent upon said feature;
applying a computational design protocol to said restricted nucleic acid to generate a plurality of variant nucleic acid sequences, said protocol excluding any variant nucleic acid sequence having said feature;
generating a plurality of nucleic acid molecules having predefined sequences corresponding to said plurality of variant nucleic acid sequences; and
screening said plurality of nucleic acid molecules for said desired property thereby to determine a variant of said restricted nucleic acid that is not subject to said patent right.

46. A method for determining a variant of a restricted nucleic acid, the method comprising:
identifying a restricted nucleic acid having a desired property, said restricted nucleic acid being subject to a patent right;
determining at least one feature of said restricted nucleic acid, wherein said patent right is contingent upon said feature;
applying a computational design protocol to said restricted nucleic acid to determine at least one portion of said restricted nucleic acid to which random mutations can be introduced, said protocol excluding any variant nucleic acid sequence having said feature;
generating a plurality of nucleic acid molecules having at least one random mutations in at least one portion of said restricted nucleic acid; and
screening said plurality of nucleic acid molecules for said desired property thereby to determine a variant of said restricted nucleic acid that is not subject to said patent right.

47. A method for determining a functional variant of a restricted protein, the method comprising:
identifying a restricted protein that exhibits a biological activity, said restricted protein being subject to a patent right;
determining at least one feature of said restricted protein, wherein said patent right is contingent upon said feature;
applying a computational design protocol to generate a plurality of variant protein sequences based on said restricted protein, said protocol excluding any variant protein sequence that correspond to a variant protein having said feature;
generating a plurality of nucleic acid molecules having predefined sequences encoding said plurality of variant proteins;
expressing said nucleic acid molecules to produce said plurality of variant proteins; and
screening said plurality of variant proteins for biological activity thereby to determine a functional variant of said restricted protein that is not subject to said patent right.

48. A method for producing an unrestricted variant protein, the method comprising:
providing a structural model and an amino acid sequence for a reference protein having an desired characteristic;
determining from the structural model and amino acid sequence at least one amino acid residue that is not correlated with said desired characteristic; and
generating at least one variant protein by introducing a mutation at said at least one amino acid residue of the reference protein,
wherein said reference protein is a restricted by a proprietary right that is contingent upon a feature of said reference protein, and
wherein said feature is altered upon mutation of said at least one amino acid residue, thereby to produce a variant protein that is unrestricted by the proprietary right.

49. The method of claim 48, further comprising screening the variant protein for the desired characteristic.

50. A method for generating a library of unrestricted variant proteins, the method comprising:
providing a structural model and an amino acid sequence for a reference protein having an desired characteristic, said reference protein being a restricted by a proprietary right that is contingent upon a feature of said reference protein;
determining from the structural model and amino acid sequence a plurality of mutation-tolerant amino acid residues that are not correlated with said desired characteristic; and
generating a plurality of variant proteins by different mutations at least a subset of said mutation-tolerant amino acid residues of the reference protein,
wherein said feature is altered upon mutation of one or more of said mutation-tolerant amino acid residues, thereby to produce a library of variant proteins that is unrestricted by the proprietary right.

51. The method of claim 50, further comprising screening the plurality of variant proteins for the desired characteristic.

52. The method of claim 51, further comprising identifying at least one of the plurality of variant proteins a desired characteristic that is substantially equivalent to the reference protein.
